(19) United States
(12) Patent Application Publication
(10) Pub. No.: US 2026/0275295 A1
(43) Pub. Date: Sep. 17, 2026

(54) LIVER ORGANOIDS WITH INTRAHEPATIC SYMPATHETIC NERVES AND METHODS OF USE THEREOF

(71) Applicant: CHILDREN'S HOSPITAL MEDICAL CENTER, Cincinnati, OH (US)

(72) Inventors: Takanori TAKEBE, Cincinnati, OH (US); Rie OUCHI, Cincinnati, OH (US)

(73) Assignee: CHILDREN'S HOSPITAL MEDICAL CENTER, Cincinnati, OH (US)

(21) Appl. No.: 19/470,904

(22) PCT Filed: May 17, 2024
(86) PCT No.: PCT/US2024/030068

§ 371 Date: Sep. 30, 2025

Related U.S. Application Data
(60) Provisional application No. 63/503,108, filed on May 18, 2023.

(51) Int. Cl. C12N 5/071; A61K 35/407

(52) U.S. Cl. CPC C12N 5/0671; A61K 35/407; C12N 2501/155; C12N 2503/02

(57) ABSTRACT

Disclosed herein are methods of producing liver organoids with intrahepatic sympathetic neurons, or sympathetic nerves, from pluripotent stem cells. These intrahepatic sympathetic neurons, or sympathetic nerves, are involved in fatty liver disease pathogenesis, and the liver organoids produced from the methods provided herein may be used as a model system for fatty liver disease and pharmaceutical screening.

### CROSS REFERENCE TO RELATED APPLICATION

The present application claims the benefit of priority under 35 U.S.C. § 119 (e) to U.S. Provisional Application No. 63/503,108, LIVER ORGANOIDS WITH INTRAHEPATIC SYMPATHETIC NERVES AND METHODS OF USE THEREOF, filed on filed May 18, 2023, which is currently co-pending herewith and which is incorporated by reference in its entirety.

### STATEMENT REGARDING FEDERALLY SPONSORED RESEARCH AND DEVELOPMENT

This invention was made with government support under DP2 DK128799 awarded by the National Institutes of Health. The government has certain rights to the invention.

### FIELD

Aspects of the present disclosure relate generally to liver organoids with intrahepatic sympathetic neurons, or sympathetic nerves, and methods of making the same from pluripotent stem cells. Additional aspects relate to using these liver organoids for disease modeling and screening.

### BACKGROUND

Fatty liver is a disease in which excess fat accumulates in the liver. The number of patients with steatohepatitis, cirrhosis, and hepatocellular carcinoma due to fatty liver is increasing worldwide, especially in developed countries, and the increased risk of cardiovascular disease in patients with simple fatty liver has been reported in recent cohort studies. However, there is still no therapeutic drug or intervention available for fatty liver. Therefore, there is a need for both robust models for drug discovery for fatty liver and development of therapeutic intervention for neglected fatty liver-related conditions.

### SUMMARY

Exemplary embodiments of the present disclosure are provided in the following numbered embodiments:

Embodiments of the disclosure encompass liver organoids, such as human liver organoids, wherein the liver organoid includes hepatic stellate cells, hepatocytes, and sympathetic neurons, or sympathetic nerves.

In some embodiments, at least about 50%, 55%, 60%, 65%, 70%, 75%, 80%, 85%, 90%, 95%, or greater, of the sympathetic neurons can be located adjacent to the hepatic stellate cells of the liver organoid. In some embodiments, the sympathetic neurons includes a sympathetic varicosity region. In some embodiments, at least about 50%, 55%, 60%, 65%, 70%, 75%, 80%, 85%, 90%, 95%, or greater, of the sympathetic varicosity region can be localized adjacent to the hepatic stellate cells of the liver organoid. In some embodiments, the sympathetic neurons further include secretory vesicles. In some embodiments, the secretory vesicles store and secrete neurotransmitters.

In some embodiments, the sympathetic neurons express: one or more neural marker, one or more sympathetic marker, one or more membrane protein marker of secretory vesicles, and/or one or more neural crest markers. In some embodiments, the one or more neural marker includes class III beta tubulin (TUBB3); the one or more sympathetic marker includes tyrosine hydroxylase (TH); the one or more membrane protein marker of secretory vesicles includes synapsin (SYN); and/or the one or more neural crest markers include SOX10, FOXD3, and/or NGFR. In some embodiments, the sympathetic neurons express TUBB3 and TH. In some embodiments, the sympathetic neurons express TUBB3, TH, and SYN, as well as SOX10, FOXD3, and NGFR.

In some embodiments, the sympathetic neurons can produce one or more sympathetic nervous system neurotransmitter and/or one or more noradrenaline synthesizing enzymes. In some embodiments, the one or more sympathetic nervous system neurotransmitter includes noradrenaline, and/or the one or more noradrenaline synthesizing enzymes include dopa decarboxylase (DDC) and/or dopamine β-hydroxylase (DBH).

In some embodiments, the sympathetic neurons can activate β-adrenergic receptor signaling in hepatic stellate cells. In some embodiments, the activation of β-adrenergic receptor signaling can be via noradrenaline. In some embodiments, the hepatic stellate cells in the liver organoid express higher levels of β-adrenergic receptor genes and lower levels of α2-adrenergic receptor genes than other cells. In some embodiments, the β-adrenergic receptor genes include ADRB1, ADRB2, and/or ADRB3, and/or the α2-adrenergic receptor genes include ADRA2A, ADRA2B, and/or ADRA2C. In some embodiments, the sympathetic neurons can suppress triglyceride (TG) accumulation in hepatocytes.

In some embodiments, the liver organoid includes spheres of epithelial cells and aggregates of mesenchymal cells. In some embodiments, the spheres of epithelial cells include hepatocytes, and the aggregates of mesenchymal cells include hepatic stellate cells and sympathetic neurons. In some embodiments, the aggregates of mesenchymal cells include a higher density of hepatic stellate cells than the spheres. In some embodiments, the cells self-assemble into the spheres of epithelial cells including hepatocytes and aggregates of mesenchymal cells including hepatic stellate cells and sympathetic neurons; there can be, in some embodiments, an observable and/or measurable boundary between the spheres of epithelial cells and aggregates of mesenchymal cells. In some embodiments, the spheres of epithelial cells including hepatocytes and aggregates of mesenchymal cells including hepatic stellate cells and sympathetic neurons self-assemble into the liver organoid containing sympathetic neurons.

In some embodiments, the liver organoid can include one or more additional cell type, such as hepatoblasts, cholangiocytes, endothelial cells, macrophages, stellate cells, Schwann cells, and/or neural crest cells. In some embodiments, the liver organoid includes a luminal structure. In some embodiments, the luminal structure includes internalized microvilli. In some embodiments, the liver organoid includes a structure with a single lumen. In some embodiments, the liver organoid does not contain hematopoietic tissue and/or acquired immune cells.

In some embodiments, the liver organoid includes about 1%-75%, 2%-65%, 5%-60%, 5-25%, or 10-20%, neural cells; about 10%-90%, 15%-75%, or 15%-65%, epithelial cells; and about 10%-90%, 15%-75%, or 15%-60%, hepatic stellate cells and hematopoietic cells. In some embodiments, the liver organoid includes at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, or greater, neural cells; wherein the liver organoid includes at least about 10%, 20%, 25%, 30%, 35%, 40%, 45%, 50%, 55%, 60%, 65%, 70%, 75%, 80%, 85%, 90%, or greater, epithelial cells; and/or wherein the liver organoid includes at least about 10%, 20%, 25%, 30%, 35%, 40%, 45%, 50%, 55%, 60%, 65%, 70%, 75%, 80%, 85%, 90%, or greater, hepatic stellate cells and hematopoietic cells. In some embodiments, the liver organoid includes at least about 1%, 2%, 3%, 4%, 5%, 6%, 7%, 8%, 9%, 10%, 11%, 12%, 13%, 14%, 15%, 16%, 17%, 18%, 19%, or 20% neural cells. In some embodiments, the liver organoid includes greater than 1% neural cells. In some embodiments, the liver organoid performs spontaneous neural activity.

In some embodiments, the liver organoid has a fatty liver phenotype. In some embodiments, the liver organoid can be induced to have a fatty liver phenotype by contacting the liver organoid having sympathetic neurons with one or more fatty acids, thereby inducing the fatty liver phenotype. In some embodiments, the one or more fatty acids include oleic acid, linoleic acid, palmitic acid, or any combination thereof, such as oleic acid alone or in combination with another fatty acid. In some embodiments, the liver organoid with a fatty liver phenotype can be determined as having levels of accumulated triglycerides. In some embodiments, the fatty liver phenotype includes accumulation of triglycerides in >5% of hepatocytes.

In some embodiments, the liver organoid is differentiated from pluripotent stem cells, such as iPSCs. In some embodiments, the liver organoid is a human liver organoid. In some embodiments, the liver organoid is an artificial liver organoid. In some embodiments, the liver organoid is three-dimensional. In some embodiments, the liver organoid is a mature liver organoid, such as, for example, a mature human liver organoid.

Further embodiments of the disclosure include methods of producing the liver organoid containing sympathetic neurons, as described above, the methods including: a) activating an FGF signaling pathway and a Wnt signaling pathway, and optionally inhibiting a BMP signaling pathway, in definitive endoderm cells (DE), for a first period of time; b) activating an FGF signaling pathway, a Wnt signaling pathway, and a retinoic acid (RA) signaling pathway, and optionally inhibiting a BMP signaling pathway, in the cells of step a), for a second period of time, thereby differentiating the DE to posterior foregut cells; and c) embedding the posterior foregut cells in a basement membrane matrix, and optionally inhibiting a BMP signaling pathway in the embedded posterior foregut cells for a third period of time; and d) culturing the posterior foregut cells for a fourth period of time to differentiate the posterior foregut cells to liver organoids; wherein a BMP signaling pathway is inhibited in step b) and/or step c).

In some embodiments, the posterior foregut cells of step c) are cultured in a hepatocyte culture medium. In some embodiments, the hepatocyte culture medium includes hepatocyte growth factor, oncostatin M, dexamethasone, or any combination thereof.

In some embodiments, the DE has been derived from pluripotent stem cells, such as, for example, embryonic stem cells and/or induced pluripotent stem cells. In some embodiments, the posterior foregut cells are in the form of spheroids and/or dissociated cells.

In some embodiments, inhibiting a BMP signaling pathway includes providing one or more BMP inhibitor. In some embodiments, inhibiting a BMP signaling pathway includes providing two or more BMP inhibitors. In some embodiments, inhibiting a BMP signaling pathway includes providing a BMP inhibitor such as, for example, Noggin, RepSox, LY364947, LDN-193189, and/or SB431542. In some embodiments, inhibiting a BMP signaling pathway includes providing LDN-193189, and/or SB431542. In some embodiments, inhibiting a BMP signaling pathway includes providing a BMP signaling pathway inhibitor at a concentration of, or of about, 50, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 450, 500, 550, 600, 650, 700, 750, 800, 850, 900, 950, 1000, 1050, 1100, 1150, 1200, 1250, 1300, 1350, 1400, 1450, 1500 nM, or any concentration within a range defined by any two of the aforementioned concentrations, including 50-1500 nM, 100-1100 nM, 100-700 nM, 200-600 nM, 150-350 nM, 200-300 nM, 100-1250 nM, 250-1250 nM, 250-1000 nM, 250-750 nM, 400-600 nM, 500-1250 nM, 750-1250 nM, or 900-1100 nM; for example, the BMP signaling pathway inhibitor can be provided at a concentration of 250 nM or about 250 nM, at a concentration of 500 nM or about 500 nM, at a concentration of 750 nM or about 750 nM, or at a concentration of 1000 nM or about 1000 nM.

In some embodiments, step c) includes inhibiting a BMP signaling pathway and further includes activating a Wnt signaling pathway. In some embodiments, the BMP signaling pathway is inhibited concurrently with activation of a Wnt signaling pathway. In some embodiments, inhibiting a BMP signaling pathway includes adding one or more BMP inhibitor, and wherein activating a Wnt signaling pathway includes adding a Wnt signaling pathway activator. In some embodiments, activating a Wnt signaling pathway includes providing a Wnt signaling pathway activator including Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, CHIR99021, CHIR 98014, AZD2858, BIO, AR-A014418, SB 216763, SB 415286, aloisine, indirubin, alsterpaullone, kenpaullone, lithium chloride, TDZD 8, and/or TWS119; for example, the Wnt signaling pathway activator can include CHIR99021. In some embodiments, activating a Wnt pathway includes providing CHIR99021. In some embodiments, activating a Wnt pathway includes providing a Wnt signaling pathway activator a concentration of, or of about, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, or 3.5 μM, or any concentration within a range defined by any two of the aforementioned concentrations, including 0.5-3.5 μM, 0.5-2 μM, 2-3.5 μM, 1-3 μM, or 1.5-2.5 μM; for example, the Wnt signaling pathway activator can be provided at a concentration of 2 μM or about 2 μM.

In some embodiments, inhibiting a BMP signaling pathway includes providing LDN-193189 and SB431542, and activating a Wnt pathway includes providing CHIR99021. In some embodiments, the BMP inhibition in step b) and/or step c) is for at least about 2 days.

In some embodiments, activating a FGF signaling pathway includes providing a FGF signaling pathway activator. In some embodiments, activating a FGF signaling pathway includes providing a FGF signaling pathway activator including FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF19, FGF20, FGF21, FGF22, and/or FGF23; for example, the FGF signaling pathway activator can include FGF4. In some embodiments, activating a FGF signaling pathway includes providing a FGF signaling pathway activator at a concentration of, or of about, 100, 150, 200, 250, 300, 350, 400, 450, 500, 550, 600, 650, 700, 750, 800, 850, 900, 950, or 1000 ng/mL, or any concentration within a range defined by any two of the aforementioned concentrations, including 100-1000 ng/mL, 100-500 ng/mL, 500-1000 ng/mL, 250-750 ng/ml, or 400-600 ng/ml; for example, the FGF signaling pathway activator can be provided at a concentration of 500 ng/ml or about 500 ng/mL.

In some embodiments, activating a RA signaling pathway includes providing a RA signaling pathway activator. In some embodiments, activating a RA signaling pathway includes providing a RA signaling pathway activator including retinoic acid, all-trans retinoic acid, 9-cis retinoic acid, CD437, EC23, BS 493, TTNPB, and AM580; for example, the RA signaling pathway activator can include retinoic acid. In some embodiments, activating a RA signaling pathway includes providing a RA signaling pathway activator at a concentration of, or of about, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.9, or 3 μM, or any concentration within a range defined by any two of the aforementioned concentrations, including 1-3 μM, 1-2 μM, 2-3 μM, or 1.5-2.5 μM; for example, the RA signaling pathway activator can be provided at a concentration of 2 μM or about 2 μM.

In some embodiments, the first period of time can be about, 0.5, 1, 2, 3, or 4 days. In some embodiments, the second period of time can be about, 0.5, 1, 2, or 3 days. In some embodiments, the third period of can be about, 0.5, 1, 2, 3, 4, 5, 6, or 7 days. In some embodiments, the fourth period of time can be about, 4, 5, 6, 7, 8, 9, 10, 11, 12, 13, 14, 15, 16, 17, 18, 19, 20, 21, 22, 23, 24, 25, 26, 27, 28, 29, or 30 days, or at least 10 days.

In some embodiments, in step c), the posterior foregut cells can be seeded in a basement membrane matrix at a seeding density of at least about 1.0×10⁵ cells, 5.0×10⁵ cells, 6.0×10⁵ cells, 7.0×10⁵ cells, 8.0×10⁵ cells, 9.0×10⁵ cells, 10.0×10⁵ cells, or higher, per μL of basement membrane matrix, or any concentration of cells per μL within a range defined by any two of the aforementioned concentrations. In some embodiments, in step c), the posterior foregut cells can be seeded in a basement membrane matrix at a seeding density of about 5.0×10⁵ cells per μL of basement membrane matrix to 10.0×10⁵ cells per μL of basement membrane matrix. In some embodiments, in step c), the posterior foregut cells can be seeded in a basement membrane matrix at a seeding density of at least about 5.0×10⁵ cells per μL of basement membrane matrix. In some embodiments, the basement membrane matrix can include Matrigel.

Further embodiments of the disclosure include cell compositions and ex vivo compositions in the form of one or more three-dimensional artificial liver organoid including hepatic stellate cells, hepatocytes, and sympathetic neurons. Such embodiments can include the liver organoid described above and/or can be produced by any of the methods described above. Embodiments of the disclosure thus include liver organoids including sympathetic neurons, artificial liver organoids including sympathetic neurons, cell compositions, and/or ex vivo compositions, as described above.

Further embodiments of the disclosure include methods of treating a liver-related disease or disorder, the methods including: transplanting, into a subject having a liver-related disease or disorder, the liver organoids including sympathetic neurons, artificial liver organoids including sympathetic neurons, cell compositions, and/or ex vivo compositions, as described above.

In some embodiments, the liver-related disease or disorder includes one or more types of liver dysfunction and/or failure, hepatitis, viral hepatitis, cholangitis, fibrosis, hepatic encephalopathy, hepatic porphyria, cirrhosis, cancer, drug-induced cholestasis, metabolic disease, autoimmune liver disease, Wilson's disease, metabolic-associated fatty liver disease, hyperammonemia, hyperbilirubinemia, Crigler-Najjar Syndrome, urea cycle disorders, Wolman disease, hepatic cancer, hepatoblastoma, metabolic dysfunction-associated steatohepatitis (MASH), metabolic dysfunction-associated liver disease (MASLD), MetALD, nonalcoholic fatty liver disease (NAFLD), drug-induced liver injury (DILI), glycogen storage disease, hemorrhagic disease, hepatic cyst, liver-related nervous system dysfunction, glucogenesis, and/or alcohol-associated liver disease. In some embodiments, the liver-related disease or disorder includes a fatty liver-related disease or disorder. In some embodiments, the fatty liver-related disease or disorder includes fatty liver disease, MASH, MASLD, NAFLD, cirrhosis, parenteral nutrition associated liver disease (PNALD), and/or cholestasis. In some embodiments, the metabolic disease includes MASH, MASLD, or NAFLD. In some embodiments, the metabolic associated fatty liver disease includes MASH. In some embodiments, the liver-related nervous system dysfunction includes MASLD, obesity, dyslipidemia, hypertension, and/or diabetes. In some embodiments, the disease or disorder includes glucogenesis.

In some embodiments, the subject has improved symptoms of biliary stricture and/or liver regeneration following transplantation. In some embodiments, the subject has an increased survival rate following transplantation. In some embodiments, the transplanted liver organoids engraft onto the liver of the subject.

Further embodiments of the disclosure include uses of the liver organoids including sympathetic neurons, artificial liver organoids including sympathetic neurons, cell compositions, and/or ex vivo compositions, as described above, as an in vitro human model system for studying hepatocyte function and developmental divergence; studying liver-related disease; identifying therapeutic targets; and/or identifying therapeutic compounds and/or compositions effective in treating a liver-related disease or disorder. Further embodiments of the disclosure include uses of the liver organoids including sympathetic neurons, artificial liver organoids including sympathetic neurons, cell compositions, and/or ex vivo compositions, as described above, for treating a liver-related disease or disorder as described above. Further embodiments of the disclosure include the liver organoids including sympathetic neurons, artificial liver organoids including sympathetic neurons, cell compositions, and/or ex vivo compositions, as described above, for use in the manufacture of a medicament for the treatment of a liver-related disease or disorder.

Further embodiments of the disclosure include methods of making a liver organoid having a reduced number of, or which is free of, sympathetic neurons, the methods including inhibiting sympathetic neuron survival in a liver organoid including sympathetic neurons, wherein said inhibition of sympathetic neuron survival ablates the sympathetic neurons from the liver organoid, thereby making a liver organoid having a reduced number of, or which is free of, sympathetic neurons.

In some embodiments, inhibiting sympathetic neuron survival includes contacting the liver organoid with an inhibitor of sympathetic neuron survival. In some embodiments, inhibiting sympathetic neuron survival includes providing the liver organoid with an inhibitor of sympathetic neuron survival including a neurotoxin. In some embodiments, the inhibitor of sympathetic neuron survival can include 6-hydroxydopamine (6-OHDA), DSP-4, MPP+, or MPTP, or any combination thereof. In some embodiments, the inhibitor of sympathetic neuron survival can include 6-OHDA. In some embodiments, the liver organoid including sympathetic neurons can be the liver organoid including sympathetic neurons, as described above.

Further embodiments of the disclosure include liver organoids having a reduced number of, or which are free of, sympathetic neurons produced by the methods including ablating the sympathetic neurons from the liver organoid, as described above. Further embodiments of the disclosure include liver organoids having a reduced number of, or which are free of, sympathetic neurons.

In some embodiments, the liver organoid having a reduced number of, or which is free of, sympathetic neurons has been subject to inhibition of sympathetic neuron survival; for example, via treatment with an inhibitor of sympathetic neuron survival; for example, wherein the inhibitor of sympathetic neuron survival is a neurotoxin; for example, wherein the inhibitor of sympathetic neuron survival comprises 6-OHDA, DSP-4, MPP+, or MPTP, or any combination thereof, such as 6-OHDA.

Further embodiments of the disclosure include methods of making a fatty liver organoid having a reduced number of, or which is free of, sympathetic neurons and including a fatty liver phenotype, the methods including contacting the liver organoid having a reduced number of, or which is free of, sympathetic neurons with one or more fatty acids, thereby inducing the fatty liver phenotype in the liver organoid having a reduced number of, or which is free of, sympathetic neurons. In some embodiments, the one or more fatty acids include oleic acid, linoleic acid, palmitic acid, or any combination thereof.

Further embodiments of the disclosure include methods of making a fatty liver organoid including a fatty liver phenotype, the methods including providing a liver organoid with an inhibitor of sympathetic neuron survival and one or more fatty acids, thereby inducing the fatty liver phenotype in the liver organoid. In some embodiments, the inhibitor of sympathetic neuron survival is a neurotoxin. Further embodiments of the disclosure include fatty liver organoids including a fatty liver phenotype made by such methods.

In some embodiments, the inhibitor of sympathetic neuron survival is selected from the group consisting of 6-hydroxydopamine (6-OHDA), DSP-4, MPP+, or MPTP, or any combination thereof. In some embodiments, the inhibitor of sympathetic neuron survival can include 6-OHDA. In some embodiments, the one or more fatty acids include oleic acid, linoleic acid, palmitic acid, or any combination thereof.

Further embodiments of the disclosure include methods including contacting a liver organoid with an activator or inhibitor of sympathetic neuron function and/or an activator or inhibitor of sympathetic neuron survival.

In some embodiments, the activator or inhibitor of sympathetic neuron function can include an activator or inhibitor of adrenergic receptor function of sympathetic neurons. In some embodiments, the activator or inhibitor of sympathetic neuron function can modulate noradrenaline production ability of the liver organoid. In some embodiments, the activator of sympathetic neuron function includes noradrenaline, isoproterenol, phenylephrine, or any combination thereof.

In some embodiments, the inhibitor of sympathetic neuron function includes prazosin, propranolol, venlafaxine, levomilnacipran, or any combination thereof. In some embodiments, the inhibitor of sympathetic neuron function or survival suppresses sympathetic neuron production and/or the elongation of sympathetic neurons. In some embodiments, the inhibitor of sympathetic neuron function or survival can include a BDNF inhibitor. In some embodiments, the BDNF inhibitor can include licochalcone A, K252a, GZD2202, cyclotraxin B, ANA12 or any combination thereof. In some embodiments, the BDNF inhibitor, such as ANA12, can be provided at a concentration of 1 μM or about 1 μM for 5 days or about 5 days on about day 15 of differentiation induction of the liver organoid.

In some embodiments, the activator of sympathetic neuron survival induces sympathetic neuron production and/or the elongation of sympathetic neurons. In some embodiments, the activator of sympathetic neuron survival includes nerve growth factor (NGF), glial cell like-derived neurotrophic factor (GDNF), brain derived neurotrophic factor (BDNF), ciliary neurotrophic factor (CNTF), netrin 1 (NTN1), anti-tumor necrosis factor alpha (TNF), or any combination thereof. In some embodiments, the activator of sympathetic neuron survival, optionally BDNF, can be provided at a concentration of 100 nM or about 100 nM for 13 days or about 13 days beginning on, or on about, day 15, optionally day 14-16, of differentiation induction of the liver organoid. In some embodiments, the inhibitor of sympathetic neuron survival includes a neurotoxin. In some embodiments, the inhibitor of sympathetic neuron survival includes 6-hydroxydopamine (6-OHDA), DSP-4, MPP+, or MPTP, or any combination thereof.

In some embodiments, the activator or inhibitor of sympathetic neuron function and/or the activator or inhibitor of sympathetic neuron survival includes an interfering RNA (RNAi) or site-directed endonuclease, optionally CRISPR/Cas9. In some embodiments, the RNAi or site-directed endonuclease targets one or more proteins and/or genes involved in noradrenaline production. In some embodiments, the RNAi or site-directed endonuclease modulates expression of the one or more proteins and/or genes involved in noradrenaline production.

In some embodiments, the liver organoid is a fatty liver organoid including a fatty liver phenotype, or the liver organoid, with or without sympathetic neurons, produced by the any of the methods described above, as appropriate.

Further embodiments of the disclosure include methods of screening for a compound or composition for the treatment of fatty liver disease, the methods including: contacting a fatty liver organoid including a fatty liver phenotype with the compound or composition; and detecting a change in the fatty liver phenotype of the fatty liver organoid.

In some embodiments, detecting the change in the fatty liver phenotype includes detecting a change in triglycerides in the fatty liver organoid after contacting with the compound or composition. In some embodiments, detecting the change in triglycerides includes detecting a reduction in triglycerides in the fatty liver organoid, thereby resulting in an improvement in the fatty liver phenotype of the fatty liver organoid. In some embodiments, detecting the change in triglycerides includes detecting an increase in triglycerides in the fatty liver organoid, thereby resulting in a worsening in the fatty liver phenotype of the fatty liver organoid. In some embodiments, detecting the change in triglycerides includes using a lipophilic fluorescent probe, optionally a BODIPY probe, BODIPY 493/503, BODIPY 558/568 C 12, or Oil red O. In some embodiments, the change in triglycerides in the fatty liver organoid is at least 5%, 10%, 15%, 20%, 25%, 30%, 35%, 40%, 45%, or 50% relative to the level of triglycerides in the fatty liver organoid before contacting with the compound or composition, optionally wherein the change in triglycerides is a reduction in triglycerides. In some embodiments, the fatty liver organoid can be the fatty liver organoid as described above.

Further embodiments of the disclosure include liver organoids as described above, and methods involving the liver organoids, as described above, where the liver organoid has been made according to a method including: a) activating an FGF signaling pathway and a Wnt signaling pathway, and optionally inhibiting a BMP signaling pathway, in definitive endoderm cells (DE), for a first period of time; b) activating an FGF signaling pathway, a Wnt signaling pathway, and a retinoic acid (RA) signaling pathway, and optionally inhibiting a BMP signaling pathway, in the cells of step a), for a second period of time, thereby differentiating the DE to posterior foregut cells; and c) embedding the posterior foregut cells in a basement membrane matrix and culturing the posterior foregut cells for a third period of time to differentiate the posterior foregut cells to liver organoids.

In some embodiments, the posterior foregut cells of step c) are cultured in a hepatocyte culture medium. In some embodiments, the hepatocyte culture medium includes hepatocyte growth factor, oncostatin M, dexamethasone, or any combination thereof. In some embodiments, the DE has been derived from pluripotent stem cells, such as embryonic stem cells and/or induced pluripotent stem cells. In some embodiments, the posterior foregut cells can be in the form of spheroids and/or dissociated cells.

In some embodiments, activating an FGF signaling pathway includes providing the cells with a FGF signaling pathway activator; optionally wherein the FGF signaling pathway activator includes FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF19, FGF20, FGF21, FGF22, and FGF23. In some embodiments, activating an FGF signaling pathway includes contacting the cells with FGF4. In some embodiments, activating an FGF signaling pathway includes providing an FGF signaling pathway activator at a concentration of, or of about, 100, 150, 200, 250, 300, 350, 400, 450, 500, 550, 600, 650, 700, 750, 800, 850, 900, 950, or 1000 ng/mL, or any concentration within a range defined by any two of the aforementioned concentrations, such as, for example, 100-1000 ng/mL, 100-500 ng/mL, 500-1000 ng/ml, 250-750 ng/mL, or 400-600 ng/mL. In some embodiments, activating an FGF signaling pathway includes providing FGF signaling pathway activator at a concentration of 500 ng/mL or about 500 ng/mL.

In some embodiments, activating a Wnt signaling pathway includes providing the cells with a Wnt signaling pathway activator; optionally wherein the Wnt signaling pathway activator comprises Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, CHIR99021, CHIR 98014, AZD2858, BIO, AR-A014418, SB 216763, SB 415286, aloisine, indirubin, alsterpaullone, kenpaullone, lithium chloride, TDZD 8, and TWS119. In some embodiments, activating a Wnt signaling pathway includes providing the cells with CHIR99021. In some embodiments, activating a Wnt signaling pathway includes providing a Wnt signaling pathway activator at a concentration of, or of about, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, or 3.5 μM, or any concentration within a range defined by any two of the aforementioned concentrations, such as, for example, 0.5-3.5 μM, 0.5-2 μM, 2-3.5 μM, 1-3 μM, or 1.5-2.5 μM. In some embodiments, activating a Wnt signaling pathway includes providing a Wnt signaling pathway activator at a concentration of 2 μM or about 2 μM.

In some embodiments, inhibiting a BMP signaling pathway includes providing the cells with a BMP signaling pathway inhibitor; optionally wherein the BMP signaling pathway inhibitor is selected from the group consisting of Noggin, RepSox, LY364947, LDN-193189, and SB431542. In some embodiments, inhibiting a BMP signaling pathway includes providing the cells with LDN-193189. In some embodiments, inhibiting a BMP signaling pathway includes providing a BMP signaling pathway inhibitor at a concentration of, or of about, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 450, 500, 550, 600, 650, 700, 750, 800, 850, 900, 950, 1000, 1050, 1100, 1150, 1200, or 1250 nM, or any concentration within a range defined by any two of the aforementioned concentrations, including 100-400 nM, 100-250 nM, 250-400 nM, 150-350 nM, 200-300 nM, 100-1250 nM, 250-1250 nM, 250-1000 nM, 250-750 nM, 400-600 nM, 500-1250 nM, 750-1250 nM, or 900-1100 nM. In some embodiments, inhibiting a BMP signaling pathway includes providing a BMP signaling pathway inhibitor at a concentration of about 100-1500 nM, about 200-1200, or about 200-1100; such as, for example, 250 nM or about 250 nM, 500 nM or about 500 nM, or 1000 nM or about 1000 nM. In some embodiments, the BMP signaling pathway is inhibited, optionally using LDN-193189, for at least about 2 days, or at least about 3 days, during the first period of time beginning on about day 3 of differentiation induction of the liver organoid.

In some embodiments, activating an RA signaling pathway includes providing the cells with a RA signaling pathway activator; optionally wherein the RA signaling pathway activator includes retinoic acid, all-trans retinoic acid, 9-cis retinoic acid, CD437, EC23, BS 493, TTNPB, and AM580. In some embodiments, activating an RA signaling pathway includes providing the cells with RA. In some embodiments, activating an RA signaling pathway includes providing an RA signaling pathway activator at a concentration of, or of about, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.9, or 3 μM, or any concentration within a range defined by any two of the aforementioned concentrations, such as, for example, 1-3 μM, 1-2 μM, 2-3 μM, or 1.5-2.5 μM. In some embodiments, the RA signaling pathway activator can be provided contacted at a concentration of 2 μM or about 2 μM.

In some embodiments, the first period of time is, or is about, 0.5, 1, 2, 3, or 4 days. In some embodiments, the second period of time is, or is about, 0.5, 1, or 2 days. In some embodiments, the third period of time is, or is about, 4, 5, 6, 7, 8, 9, 10, 11, 12, 13, 14, 15, 16, 17, 18, 19, 20, 21, 22, 23, 24, 25, 26, 27, 28, 29, or 30 days, or at least 4 days.

In some embodiments, the posterior foregut cells can be embedded in the basement membrane matrix at a concentration of or of about 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 10×10⁵ cells, or more, per μL of basement membrane matrix, or any concentration of cells per μL within a range defined by any two of the aforementioned concentrations, for example, 5×10⁴-5×10⁵, 5×10⁴-1×10⁵, 1×10⁵-10×10⁵, 4×10⁵-9×10⁵, 5×10⁵-8×10⁵, or 8×10⁴-3×10⁵ cells per μL of basement membrane matrix. In some embodiments, the basement membrane matrix is Matrigel.

In some embodiments, the liver organoid can be derived from a patient, such as, for example, a patient having a liver disease.

In some embodiments, the liver organoids, artificial liver organoids, cell compositions, and/or ex vivo compositions, as described herein, can have sympathetic neurons present in a pre-determined amount. In some embodiments, the pre-determined amount of sympathetic neurons can be achieved by using the methods described herein to produce sympathetic neurons alone, or in combination with the methods described herein to inhibit formation of sympathetic neurons, such that a pre-determined amount, or range, or percentage, of sympathetic neurons can be produced.

Further embodiments of the disclosure include methods of screening for a positive therapeutic response to administration of a treatment for a liver-related disease or disorder, the methods including: subjecting the liver organoid, artificial liver organoid, cell composition, and/or ex vivo composition according to any one of claim 1-41, 74-78, 91, 98-100, 104, 110, or 137-165, to a treatment for the liver-related disease or disorder, wherein the liver organoid, artificial liver organoid, cell composition, and/or ex vivo composition has been derived from iPSCs obtained from a subject; and detecting a change in a phenotype of the liver organoid, artificial liver organoid, cell composition, and/or ex vivo composition.

Further embodiments of the disclosure include kits including means for performing any of the methods described herein. Further embodiments of the disclosure include kits including one or more liver organoids, artificial liver organoids, cell compositions, and/or ex vivo compositions, as described herein. For example, kits according to the disclosure can include one or more type of culturing medium, and one or more signaling pathway activator or signaling pathway inhibitor, and other components, as appropriate for the method. In some embodiments, the kits can further include one or more FGF signaling pathway activator, one or more Wnt signaling pathway activator, one or more RA signaling pathway activator, and one or more BMP signaling pathway inhibitor. In some embodiments, the kits can further include iPSCs, PSCs, and/or posterior foregut cells and/or posterior foregut endoderm cells. In some embodiments, the kits can further include a culture medium, such as, for example, a hepatocyte culture medium.

### BRIEF DESCRIPTION OF THE DRAWINGS

In addition to the features described herein, additional features and variations will be readily apparent from the following descriptions of the drawings and exemplary embodiments. It is to be understood that these drawings depict embodiments and are not intended to be limiting in scope.

FIG. 1 depicts an embodiment of immunofluorescence microscopy images of a human liver organoid differentiated from iPSCs and which is positive for class III beta tubulin (TUBB3), which is a neuronal marker, and tyrosine hydroxylase (TH), which is a sympathetic neuronal marker, indicating the presence of sympathetic neurons in the liver organoids. The upper left-hand panel, figure,

FIG. 2 depicts an embodiment of immunofluorescence microscopy images of a human liver organoid differentiated from iPSCs. The arrows indicate the presence of TUBB3-positive varicosity regions of the sympathetic neurons, or sympathetic nerves, as well as the presence of synapsin (SYN) in a speckle-like pattern, which indicates secretory vesicles.

FIG. 3 depicts an embodiment of immunofluorescence microscopy images of a human liver organoid differentiated from iPSCs and having sympathetic neurons expressing dopa decarboxylase (DDC) and dopamine β-hydroxylase (DBH), which are noradrenaline synthesizing enzymes.

FIG. 4 depicts an embodiment of an immunofluorescence microscopy image and quantification of a human liver organoid expressing the fluorescent calcium sensor GCaMP, and detection of fluorescent activity at regions corresponding to the location of sympathetic neurons, or sympathetic nerves, showing the regions where the nerve was and was not localized.

FIG. 5 depicts an embodiment of an immunofluorescence microscopy image and quantification of a human liver organoid stained with the sympathetic neuronal marker TH, the hepatic stellate cell marker vimentin (VIM), and hepatocyte marker E-cadherin (E-cad), with arrows showing that the majority of the sympathetic neurons are localized adjacent to hepatic stellate cells.

FIG. 6 depicts an embodiment of a quantification of CAMP concentration in liver organoids treated with the sympathetic neuron toxin 6-hydroxydopamine (6-OHDA), noradrenaline (norepinephrine; “Nore”), or the β-adrenergic receptor agonist isoproterenol (ISO).

FIG. 7 depicts an embodiment of quantitative rt-PCR of β-adrenergic receptor genes (ADRB1, ADRB2, and ADRB3) and α2-adrenergic receptor genes (ADRA2A, ADRA2B, and ADRA2C) between hepatocytes and stellate cells in liver organoids. Liver organoids differentiated from two iPSC lines (1231A3 and 1383D6) were examined.

FIGS. 8A-C depict embodiments of characterizing liver organoids that have been ablated of sympathetic neurons, or sympathetic nerves, with 6-OHDA. FIG. 8A depicts immunofluorescence microscopy images of liver organoids treated with or without 6-OHDA and with or without oleic acid stained with BODIPY 493/503, and quantification of triglyceride (TG) accumulation of the liver organoids. FIG. 8B depicts RT-qPCR quantification of the gluconeogenesis genes G6PC and PCK1, and lipogenesis genes CD36 and SCD in 6-OHDA-treated liver organoids compared to control. FIG. 8C depicts RT-qPCR quantification of the cytokines CXCL8 and TNF in liver organoids treated with or without 6-OHDA and with or without oleic acid.

FIGS. 9A-B depict embodiments of brightfield and immunofluorescence microscopy images of liver organoids (day 20 after iPSC differentiation and liver induction) produced by the methods disclosed herein compared to previous methods. FIG. 9A shows brightfield images of liver organoids produced by both methods. FIG. 9B show immunofluorescence images of liver organoids produced by both methods and stained with TUBB3 and E-cad, showing that TUBB3-positive sympathetic neurons are more abundant in liver organoids produced by the methods disclosed herein.

FIG. 10 depicts an embodiment of real-time PCR quantification of SOX10, PAX6, TUBB3, ASCL1, and TH gene expression during the process of human liver organoid production from iPS cells.

FIGS. 11A-B depicts embodiments of immunofluorescence microscopy images of human liver organoids (day 20 after iPSC differentiation and liver induction) stained for TUBB3 and SOX10. FIG. 11A shows that SOX10+ neural crest-like cells were always present around TH+/TUBB3+ sympathetic-like nerves. FIG. 11B shows that TH−/TUBB3+ non-sympathetic-like nerves were produced when the number of SOX10+ neural crest-like cells was low.

FIG. 12 depicts an embodiment of real-time PCR quantification of neural crest marker expression in foregut spheroids treated with LDN193189 on day 6 of differentiation.

FIG. 13 depicts embodiments of single cell RNA sequencing (scRNAseq) and real-time PCR quantification of gene expression in liver organoids, indicating that neuron attractive factor BDNF was highly expressed in hepatic stellate-like cells of the liver organoids.

FIG. 14 depicts embodiments of immunofluorescence microscopy images and volume analysis of human liver organoids (day 20 after iPSC differentiation and liver induction) treated with and without ANA12 on day 15 of differentiation.

FIGS. 15A-B depicts embodiments of real-time PCR quantification and immunofluorescence microscopy images of human liver organoids treated with and without LDN-193189 on days 3-6 of differentiation. FIG. 15A depicts real-time PCR quantification of autonomic marker ASCL1 expression in LDN-193189-treated human liver organoids on day 20 of differentiation induction. FIG. 15B depicts immunofluorescence microscopy images of LDN-193189-treated human liver organoids on day 20 of differentiation induction stained for TUBB3 and TH.

FIG. 16 depicts embodiments of immunofluorescence microscopy images of human liver organoids on day 28 of differentiation induction, treated with and without BDNF for on days 15-28 of differentiation, stained for TUBB3.

FIGS. 17A-E depict results from comparing the production of neural crest cells, TH/TUBB+ sympathetic neurons and liver production via BMP signaling pathway inhibition (e.g. using LDN193189 at 250 nM, 500 nM and 1000 nM, and without LDN for comparison), including expression of neural markers. FIG. 17A depicts a schematic workflow of an exemplary process for inducing neural crest cell and sympathetic neurons, or sympathetic nerves, in hiPSC derived-HLOs. FIG. 17B depicts images of pFG (Day 6) with LDN-free, 250 nM LDN, 500 nM LDN, or 1000 nM LDN. FIG. 17C depicts results from flow cytometry analysis of CD57 and NGFR in spheroids at Day 14 with LDN-free, 250 nM LDN, 500 nM LDN, or 1000 nM LDN culture. FIG. 17D depicts whole mount staining of SOX10 in spheroids at Day 10 with LDN-free, 250 nM LDN, or 500 nM LDN culture. FIG. 17E depicts gene expression of neural crest markers in spheroids at Day 6-10 with LDN-free, 250 nM LDN, 500 nM LDN, or 1000 nM LDN culture.

FIGS. 18A-E depict additional results from comparing the production of neural crest cells, TH/TUBB+ sympathetic neurons and liver production under LDN193189 250 nM, 500 nM and 1000 nM, and without LDN, including expression of neural markers. FIG. 18A depicts images of HLO with LDN-free, 250 nM LDN, 500 nM LDN, or 1000 nM LDN culture. FIG. 18B depicts gene expression of neural crest markers and neural associated markers during HLO induction from two different iPS cells. FIG. 18C depicts whole mount staining of SOX10 and TUBB3 or SOX10 and TH in HLO at day 20 with LDN-free, 250 nM LDN, 500 nM LDN, or 1000 nM LDN culture. FIG. 18D depicts whole mount staining of TH in HLO at day 20 with LDN-free, 250 nM LDN, 500 nM LDN, or 1000 nM LDN culture. FIG. 18E depicts gene expression of TH in HLO at day 20 with LDN-free, 250 nM LDN, 500 nM LDN, or 1000 nM LDN culture.

FIGS. 19A-B depict additional results from comparing the production of neural crest cells, TH/TUBB+ sympathetic neurons and liver production under LDN193189 250 nM, 500 nM and 1000 nM, and without LDN, including expression of hepatocyte markers, sympathetic innervation, and albumin secretion. FIG. 19A depicts gene expression of hepatocyte markers with LDN-free, 250 nM LDN, 500 nM LDN, or 1000 nM LDN culture in HLO derived from two different iPS lines (1231A3 and 1383D6). FIG. 19B depicts albumin (ALB) secretion per well of a 24 well plate (1 day supernatants after media change).

FIGS. 20A-D depict comparative results when BMP signaling pathway inhibition (e.g. via LDN) is used in combination with Wnt signaling pathway activation (e.g. via SB). FIG. 20A depicts a brightfield image of HLO at day 15 under LDN, LDN and SB, or SB culture (Day 3-Day 6 addition). FIG. 20B depicts whole mount staining of HLO at Day 15 under LDN and SB or SB only (day3-day6 addition). FIG. 20C depicts a brightfield image of HLO at day 20 under LDN with 2 μM or 3 μM CHIR culture (Day 3-Day 6 addition). FIG. 20D depicts whole mount staining of HLO at day 20 under LDN with 2 μM or 3 μM CHIR culture (Day 3-Day 6 addition).

FIG. 21 depicts a comparison of media culture conditions and methods evaluated.

FIGS. 22A-B depict results demonstrating that hepatic stellate cells are the target cells of the sympathetic nervous system within the HLO, rather than hepatocytes. FIG. 22A depicts whole mount staining of TUBB3, Vimentin, ECAD in HLO from two different iPS cells. Arrows indicate binding sites on VIM+ and TUBB3+ cells. This sits are also defined as binding sites in quantitative analysis. FIG. 22B depicts whole mount staining of TUBB3, TH, and ECAD in 250 nM LDN-treated HLO at day 20.

FIGS. 23A-B depict results showing the involvement of specific cell types at various stages of differentiation. FIG. 23A depicts a brightfield image of HLO at Day 15 and Day 20. FIG. 23B depicts flow-cytometory of NGFR+/CD57+ neural crest cells at Day 15 and Day 20.

FIGS. 24A-B depict results showing that were neural cells were enriched in liver stellate cells in abundant aggregates. FIG. 24A depicts whole mount staining of mCherry and TH in HLO derived from TUBB3-mCherry reporter iPS cells. FIG. 24B depicts tracing of mCherry expression from Day 13 to Day 20 in HLO.

FIG. 25 depicts the effect of OA treatment in increasing the amount of TH+ cells in HLO derived from two different iPS lines, showing an increase in innervation in a NASH model.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The following description of various embodiments is exemplary and explanatory only and is not to be construed as limiting or restrictive in any way. Other embodiments, features, objects, and advantages of the present teachings will be apparent from the description and accompanying drawings, and from the claims.

The disclosure herein uses affirmative language to describe the numerous embodiments. The disclosure also includes embodiments in which subject matter is excluded, in full or in part, such as substances or materials, method steps and conditions, protocols, or procedures.

It should be understood that any use of subheadings herein are for organizational purposes, and should not be read to limit the application of those subheaded features to the various embodiments herein. Each and every feature described herein is applicable and usable in all the various embodiments discussed herein and that all features described herein can be used in any contemplated combination, regardless of the specific example embodiments that are described herein. It should further be noted that exemplary description of specific features are used, largely for informational purposes, and not in any way to limit the design, subfeature, and functionality of the specifically described feature.

### Overview

As described herein, liver organoids having sympathetic neurons, in addition to hepatic stellate cells and hepatocytes, have been developed, along with methods of making and using the same. Prior to the present disclosure, liver organoids having neural cells and/or functionality had not been produced, or demonstrated, in any significant way. The presently described liver organoids having sympathetic neurons allow for a system which more closely resembles that of the human liver and liver tissue developed in vivo.

In recent years, several mouse studies have shown that sympathetic nerves projecting to the liver (intrahepatic sympathetic nerves) contribute to the pathogenesis of fatty liver. Since sympathetic nerves have also been observed in human liver, intrahepatic sympathetic nerves have begun to attract attention as a new therapeutic target for fatty liver. On the other hand, because the localization of intrahepatic sympathetic nerves differs between mice and humans, and their target cells are also different, it has heretofore been unclear whether human intrahepatic sympathetic nerves act on fatty liver. Even if they do, it is questionable whether human intrahepatic sympathetic nerves have the same accelerating effect on fatty liver as mouse intrahepatic sympathetic nerves. In particular, hepatic stellate cells, a type of mesenchymal cell that constitutes the liver, are specifically targeted by the human intrahepatic sympathetic nerves and are known to produce growth factors involved in the onset and progression of fatty liver.

Despite this, there has been no demonstration to date of the existence of a human intrahepatic sympathetic nerve-specific mechanism of action via hepatic stellate cells in fatty liver. In addition, no liver model with sympathetic innervation targeting hepatic stellate cells has been demonstrated to date, and the action of human intrahepatic sympathetic nerves via hepatic stellate cells in fatty liver has been unclear.

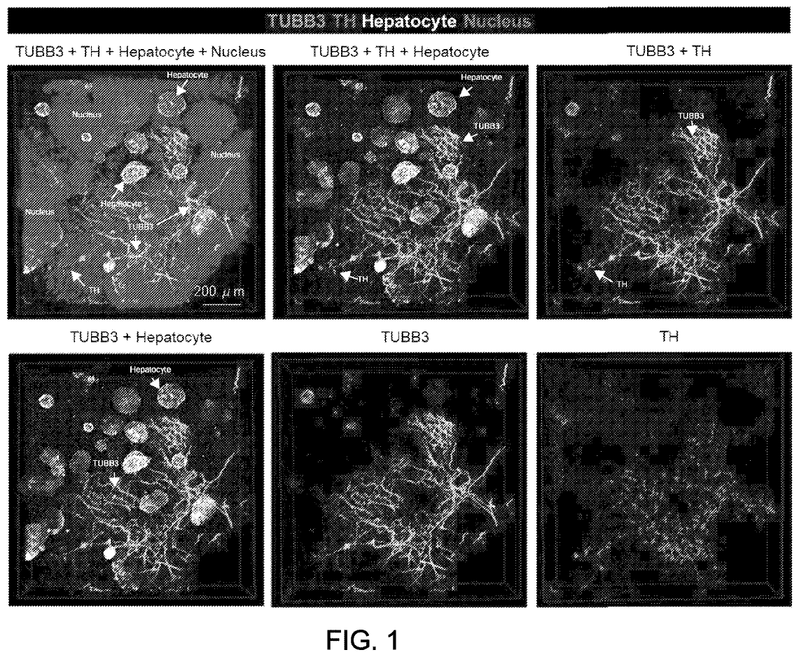

Human liver organoids (HLOs), which are liver-like three-dimensional tissues containing multiple lineage cells such as hepatic stellate cells and hepatocytes, and methods of making the same have been previously disclosed. These liver organoids have also been used in models to reproduce the pathology of fatty liver. Accordingly, to study the human intrahepatic sympathetic nervous system in the liver, HLOs containing sympathetic neurons, or sympathetic nerves (which additionally include Schwann cells and other cells of the nerve), in human liver organoids were developed and characterized (FIG. 1), as described herein, and the effects of human intrahepatic sympathetic nerves on fatty liver pathogenesis were investigated.

Further, it was found that sympathetic neurons, or sympathetic nerves, in human liver organoids: (1) produce noradrenaline and perform spontaneous neural activity, (2) target hepatic stellate cells as well as human liver internal sympathetic nerves, and (3) optionally activate β-adrenoreceptor signaling in hepatic stellate cells. In addition, sympathetic neurons, or sympathetic nerves, in human liver organoids suppress triglyceride (TG) accumulation in hepatocytes. The ability of human liver organoids with sympathetic neurons, or sympathetic nerves, to serve as a useful human liver model to test the effects of sympathetic neurons, or sympathetic nerves, in human liver on fatty liver was thus demonstrated.

These findings and the human liver organoids described herein allow for the elucidation of the mechanisms involved in human liver development and the mechanisms of action of various conditions. For example, the human liver organoids described herein can be used to study human hepatic sympathetic innervation in various conditions, such as, for example, liver diseases, glucogenesis, and the like, including assessing the features and functions of sympathetic nerves that are altered by the condition, such as liver disease, glucogenesis, and the like.

Additional studies described herein demonstrate the ability to remove, ablate, or inhibit the formation of neural cells. This allows for the ability to construct liver organoids with a bespoke amount of neural cells and to artificially regulate neural activity.

The human liver organoids described herein additionally have value in treatment of various diseases. For example, these human liver organoids can be used as an alternative source of liver tissue for liver transplantation. This can be particularly helpful to reduce the risk of developing diabetes. The human liver organoids can be derived from the same subject being treated.

The human liver organoids described herein additionally can be used in screening for compounds or compositions to be used in treating various conditions, such as, for example, nerve-targeted liver diseases.

Further details are provided in the sections that follow.

### Definitions of Terms

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, technical and scientific terms used herein have the same meaning as commonly understood when read in light of the instant disclosure by one of ordinary skill in the art to which the present disclosure belongs. For purposes of the present disclosure, the following terms are explained below.

The disclosure herein uses affirmative language to describe the numerous embodiments. The disclosure also includes embodiments in which subject matter is excluded, in full or in part, such as substances or materials, method steps and conditions, protocols, or procedures.

As used herein the specification, “a” or “an” may mean one or more. As used herein in the claim(s), when used in conjunction with the word “comprising,” the words “a” or “an” may mean one or more than one. Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

By “about” is meant a quantity, level, value, number, frequency, percentage, dimension, size, amount, weight or length that varies by as much as 10% to a reference quantity, level, value, number, frequency, percentage, dimension, size, amount, weight or length.

The use of the term “or” in the claims is used to mean “and/or” unless explicitly indicated to refer to alternatives only or the alternatives are mutually exclusive, although the disclosure supports a definition that refers to only alternatives and “and/or.” For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment. As used herein “another” may mean at least a second or more.

The term “ones” means more than one.

As used herein, the term “plurality” may be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in various embodiments.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

As used herein, the terms “treatment,” “treating,” “treat,” and the like, with respect to a disease or condition, can refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. For example, a treatment can include executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

“Treatment,” as used herein, thus can cover any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease and/or relieving one or more disease symptoms. “Treatment” can also encompass delivery of an agent or administration of a therapy in order to provide for a pharmacologic effect, even in the absence of a disease or condition.

The term “therapeutically effective” or “therapeutically effective amount” as used throughout this application can refer to an amount effective to achieve a desired and/or beneficial effect, and/or anything that promotes or enhances the well-being of the subject with respect to the medical treatment of a condition. This includes, but is not limited to, a reduction in the frequency or severity of one or more signs or symptoms of a disease. An effective amount can be administered in one or more administrations. In the methods, a therapeutically effective amount is an amount appropriate to treat an indication. By treating an indication is meant achieving any desirable effect, such as one or more of palliate, ameliorate, stabilize, reverse, slow, or delay disease progression, increase the quality of life, or to prolong life. Such achievement can be measured by any suitable method, such as measurement of tumor size or blood cell count, or any other suitable measurement.

The terms “effective amount” or “effective dose” as used herein have their plain and ordinary meaning as understood in light of the specification, and refer to that amount of a recited composition or compound that results in an observable effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The term “disease state” as used herein, can generally refer to a condition that affects the structure or function of an organism. Disease states can include, for example, stages of a disease progression.

As used herein, the term “assessing” can include any form of measurement, and includes determining if an element is present or not. The terms “determining,” “measuring,” “evaluating,” “assessing” and “assaying” can be used interchangeably and can include quantitative and/or qualitative determinations.

As used herein, the terms “modulated” or “modulation,” or “regulated” or “regulation” and “differentially regulated” can refer to both up regulation (i.e., activation or stimulation, e.g., by agonizing or potentiating) and down regulation (i.e., inhibition or suppression, e.g., by antagonizing, decreasing or inhibiting), unless otherwise specified or clear from the context of a specific usage.

As used herein, the term “marker” or “biomarker” can refer to any measurable substance taken as a sample from a subject whose presence is indicative of some phenomenon. Non-limiting examples of such phenomenon can include a disease state, a condition, or exposure to a compound or environmental condition. In various embodiments described herein, biomarkers may be used for diagnostic purposes (e.g., to diagnose a disease state, a health state, an asymptomatic state, a symptomatic state, etc.). The term “biomarker” may be used interchangeably with the term “marker”. The term “marker” or “biomarker” can include a biological molecule, such as, for example, a nucleic acid, peptide, protein, hormone, and the like, whose presence or concentration can be detected and correlated with a known condition, such as a disease state. It can also be used to refer to a differentially expressed gene whose expression pattern can be utilized as part of a predictive, prognostic or diagnostic process in healthy conditions or a disease state, or which, alternatively, can be used in methods for identifying a useful treatment or prevention therapy.

As used herein, the term “cellular phenotype” can refer to any determinable, observable, and/or measurable characteristic associated with a cell population.

As used herein, a “model” can include one or more in vitro or in vivo disease models; a model can also include algorithms, one or more mathematical techniques, one or more machine learning algorithms, or a combination thereof. A model can be used in a process and/or applied to an assay, in accordance with various embodiments as disclosed herein.

As used herein, a “process” can include one or more steps involving one or more features of one or more model as disclosed herein.

The terms “function” and “functional” as used herein have their plain and ordinary meaning as understood in light of the specification, and refer to a biological, enzymatic, or therapeutic function.

The term “inhibit” as used herein has its plain and ordinary meaning as understood in light of the specification, and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

The terms “nucleic acid” or “nucleic acid molecule” as used herein have their plain and ordinary meaning as understood in light of the specification, and refer to polynucleotides, such as deoxyribonucleic acid (DNA) or ribonucleic acid (RNA), oligonucleotides, those that appear in a cell naturally, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, retrovirus, lentivirus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein has its plain and ordinary meaning as understood in light of the specification and refers to a sequence being after the 3′-end of a previous sequence, on the strand containing the encoding sequence (sense strand) if the nucleic acid is double stranded. The term “upstream” on a nucleic acid as used herein has its plain and ordinary meaning as understood in light of the specification and refers to a sequence being before the 5′-end of a subsequent sequence, on the strand containing the encoding sequence (sense strand) if the nucleic acid is double stranded. The term “grouped” on a nucleic acid as used herein has its plain and ordinary meaning as understood in light of the specification and refers to two or more sequences that occur in proximity either directly or with extra nucleic acids in between, e.g. linkers, repeats, or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

The term “purity” of any given substance, compound, or material as used herein has its plain and ordinary meaning as understood in light of the specification and refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity can be affected by unwanted impurities, including but not limited to nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of host cell proteins, host cell nucleic acids, plasmid DNA, contaminating viruses, proteasomes, host cell culture components, process related components, mycoplasma, pyrogens, bacterial endotoxins, and adventitious agents. Purity can be measured using technologies including but not limited to electrophoresis, SDS-PAGE, capillary electrophoresis, PCR, rtPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 85, 90, 91, 92, 93, 94, 95, 96, 97, 98, 99, or 100% of the expected overall amount, including all decimals in between. Yield can be affected by the efficiency of a reaction or process, unwanted side reactions, degradation, quality of the input substances, compounds, or materials, or loss of the desired substance, compound, or material during any step of the production.

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or “carrier” can refer to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), and PLURONICS®. The composition, if desired, can also contain minor amounts of wetting, bulking, emulsifying agents, or pH buffering agents. These compositions can take the form of solutions, suspensions, emulsion, sustained release formulations and the like. The formulation should suit the mode of administration.

Cryoprotectants are cell composition additives to improve efficiency and yield of low temperature cryopreservation by preventing formation of large ice crystals. Cryoprotectants include but are not limited to DMSO, ethylene glycol, glycerol, propylene glycol, trehalose, formamide, methyl-formamide, dimethyl-formamide, glycerol 3-phosphate, proline, sorbitol, diethyl glycol, sucrose, triethylene glycol, polyvinyl alcohol, polyethylene glycol, or hydroxyethyl starch. Cryoprotectants can be used as part of a cryopreservation medium, which include other components such as nutrients (e.g. albumin, serum, bovine serum, fetal calf serum [FCS]) to enhance post-thawing survivability of the cells. In these cryopreservation media, at least one cryoprotectant may be found at a concentration that is, is about, is at least, is at least about, is not more than, or is not more than about, 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90%, or any percentage within a range defined by any two of the aforementioned numbers.

Additional excipients with desirable properties include but are not limited to preservatives, adjuvants, stabilizers, solvents, buffers, diluents, solubilizing agents, detergents, surfactants, chelating alcohols, agents, antioxidants, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in composition at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular, subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration.

As used herein, a “carrier” has its plain and ordinary meaning as understood in light of the specification and refers to a compound, particle, solid, semi-solid, liquid, or diluent that facilitates the passage, delivery and/or incorporation of a compound to cells, tissues and/or bodily organs.

As used herein, a “diluent” has its plain and ordinary meaning as understood in light of the specification and refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The term “% w/w” or “% wt/wt” as used herein has its plain and ordinary meaning as understood in light of the specification and refers to a percentage expressed in terms of the weight of the ingredient or agent over the total weight of the composition multiplied by 100. The term “% v/v” or “% vol/vol” as used herein has its plain and ordinary meaning as understood in the light of the specification and refers to a percentage expressed in terms of the liquid volume of the compound, substance, ingredient, or agent over the total liquid volume of the composition multiplied by 100.

The term “basement membrane matrix” or “extracellular matrix” as used herein has its plain and ordinary meaning in light of the specification and refers to any biological or synthetic compound, substance, or composition that enhances cell attachment and/or growth. Any extracellular matrix, as well as any mimetic or derivative thereof, known in the art can be used for the methods disclosed herein. Some examples of extracellular matrices, or mimetics or derivative thereof, include but are not limited to cell-based feeder layers, polymers, proteins, polypeptides, nucleic acids, sugars, lipids, poly-lysine, poly-ornithine, collagen, collagen IV, gelatin, fibronectin, vitronectin, laminin, laminin-511 elastin, tenascin, heparan sulfate, entactin, nidogen, osteopontin, perlecan, fibrin, basement membrane, Matrigel®, hydrogel, PEI, WGA, or hyaluronic acid, or any combination thereof. A common basement membrane matrix that is used in laboratories are those isolated from murine Engelbreth-Holm-Swarm (EHS) sarcoma cells. However, these basement membrane matrices are derived from non-human animals and therefore contain xenogeneic components that prevent its use towards humans. They are also not defined, which can lead to variability in manufacturing, as well as potentially harbor pathogens. Accordingly, in some embodiments, the methods for culturing cells may involve the use of synthetic and/or defined alternatives to these xenogeneic basement membrane matrices. The use of non-xenogeneic basement membrane matrices or mimetics or derivatives thereof enables manufacturing of biological products better suited for human use.

The term “sympathetic nerve”, “sympathetic nervous system”, and “sympathetic neuron” as used herein have their plain and ordinary meaning as understood in light of the specification and refer to the nervous system and components thereof belonging to the autonomic nervous system and involved in regulating the unconscious behavior of the body such as maintaining homeostasis and hormonal response. The sympathetic nervous system in the liver play an innate role in liver function, including nutrient metabolism and controlling blood and bile flow. In view of these roles, abnormal sympathetic nerve function in the liver have been attributed to liver disorders such as steatohepatitis and fibrosis. As provided herein, a characteristic of sympathetic neurons are the presence of sympathetic varicosities (bulbs or bulges marking the synaptic area).

The terms “passage” and “passaging” as used herein have their plain and ordinary meaning as understood in light of the specification, and refer to the conventional approaches performed in biological cell culture methods to maintain a viable population of cells for prolonged periods of time. As cells are generally proliferative in cell culture, they undergo multiple cycles of mitosis until occupying the available space, which is typically a surface of a cell culture container (e.g., a plate, dish, or flask) submerged under culture medium. For example, the cells may grow out as a monolayer on a cell culture container surface. If the growing cells occupy the entire available space of surface, they cannot proliferate further and may exhibit senescent behavior. In order to continue growth of the cells, which may be performed to maintain the viability and proliferative nature of the cells and/or to expand the number of cells for downstream purposes, the cells may be passaged by taking a fraction of the cells and seeding this fraction onto a fresh surface (e.g., of a cell culture container) in culture medium. This fraction of the cells will continue to proliferate and multiply until they occupy the available space of the new surface, upon which this passaging can be repeated successively.

The microscopic architecture of the liver is made up of polygonal structures called “hepatic lobules”. Classically, these lobules take on a hexagonal structure, although other geometric shapes are observed depending on tissue specification. Each lobule unit comprises plates or layers of hepatocytes surrounding an internal central vein and encapsulated by bundles of vessels called portal triads, which are made up of a portal vein, hepatic artery, and bile duct. Hepatic activity occurs as blood flows from the portal triads at the periphery, across the hepatocytes, and into the central vein to return to the circulatory system. Due to the asymmetric organization of these lobules, the layers of hepatocytes are divided into three zones. Cells in the “periportal zone” (zone 1) are closest to the portal triad and receive the most oxygenated blood, the pericentral zone (zone 3) are closest to the central vein and therefore receive the least amount of oxygenated blood, and the transition zone (zone 2) is in between zone 1 and 3. Due to this separation, each zone of hepatocytes exhibit differing activities. For example, zone 1 hepatocytes are involved in oxidative liver functions such as gluconeogenesis and oxidative metabolism of fatty acids, whereas zone 3 hepatocytes are involved in glycolysis, lipogenesis, and cytochrome P450-mediated detoxification. In some embodiments, the liver organoids disclosed herein exhibit a periportal-like identity resembling the tissue found in the periportal zone of liver lobules, including the functional and cellular marker characteristics of the periportal zone.

The term “bilirubin” as used herein has its plain and ordinary meaning as understood in light of the specification and refers to the naturally occurring metabolite created by normal catabolic degradation of heme. Bilirubin arises from the catalysis of biliverdin by biliverdin reductase. In the liver, bilirubin is conjugated with glucuronic acid by a family of enzymes called UDT-glucuronosyltransferases (UGTs). This conjugation renders bilirubin water soluble, enabling it to be carried in bile to the small intestine and colon, whereby it is further metabolized to waste products. Dysfunctional bilirubin metabolism, particularly due to abnormal function of UGTs preventing conjugation of bilirubin, leads to accumulation of bilirubin and is associated with various diseases characterized by hyperbilirubinemia. Notably, however, while excessive bilirubin is detrimental, bilirubin also has antioxidant capabilities and therefore may have beneficial effects in reducing oxidative damage in cells.

The term “hyperbilirubinemia” as used herein has its plain and ordinary meaning as understood in light of the specification and refers to the condition of elevated levels of bilirubin, which is a natural product of heme catabolismilirubin is filtered from the blood by the liver and is converted to water soluble intermediates, which are then released to the intestinal tract in bile, metabolized by microbiota, and excreted as waste. In neonates, bilirubin levels, which were originally cleared by the mother through the placenta, might not be adequately cleared by the immature liver. Excessive levels of bilirubin may potentially cause severe neurological damage (kernicterus). In adults, hyperbilirubinemia may also result from diseases affecting the liver, such as hepatitis and cirrhosis. Neonatal hyperbilirubinemia is treated by phototherapy, or with blood transfusion in extreme cases, whereas treatments in adults are directed to the underlying cause.

The term “L-gulonolactone oxidase” and “GULO” as used herein has its plain and ordinary meaning as understood in light of the specification and refers to the enzyme that catalyzes L-gulonolactone to produce L-xylo-hex-3-gulonolactone and hydrogen peroxide. The L-xylo-hex-3-gulonolactone then spontaneously converts to ascorbate (vitamin C). Accordingly, this enzyme is involved in the biosynthesis of vitamin C, which is an essential nutrient that is involved in many biological functions such as use as a cofactor for several important enzymes and as an antioxidant. Notably, humans, as well as other haplorrhine primates, certain species of bats, and Guinea pigs have evolved to harbor a non-functional GULO gene. Therefore, these organisms are unable to synthesize ascorbate and require vitamin C intake from diet or supplementation, where a deficiency of vitamin C can lead to scurvy. As applied to the disclosure herein, a “functional GULO protein” is a GULO protein that has L-gulonolactone catalytic activity to result in the production of ascorbate. Conversely, an “inactive” GULO protein or “non-functional” GULO protein is one that does not have the catalytic activity to produce ascorbate. Humans and cells that are derived from humans comprise a non-functional GULO protein and do not have the ability to synthesize ascorbate. However, as disclosed herein, human cells may be engineered to express a functional GULO protein to enable ascorbate synthesis ability. These functional GULO proteins may be expressed in human cells (or other cells that are unable to normally synthesize ascorbate) through conventional methods of cloning, such as genetically engineering cells to have genetic sequences that encode for a functional GULO protein.

The term “exogenous” as used herein has its plain and ordinary meaning as understood in light of the specification and refers to external factors that originate outside of a biological specimen (e.g., a cell, population of cells, organoid, etc.), as opposed to being naturally occurring and/or produced by the biological specimen itself. As used herein, exogenous components, reagents, and/or conditions, are components, reagents, and/or conditions that are added to compositions described herein, although this does not necessarily preclude the possibility of the same components, reagents, and/or conditions also being present through a function endogenous to a biological specimen.

The terms “liver organoid” and “hepatocyte organoid” are used interchangeably herein, and refer to populations of cells differentiated in vitro to form self-organizing structures, which generally are three-dimensional (3D), and include one or more functional cell types. Liver organoids differ from naturally occurring liver tissue in a number of ways. For example, as compared with naturally occurring liver tissue, liver organoids can have a structure having a single lumen and generally a spherical shape, and can include a basement membrane which is unnatural. The single lumen of a liver organoid contains 3D tissues but generally does not make any hepatic lobular structure nor cord-like structure, as with naturally occurring liver tissue. Liver organoids also generally do not contain hematopoietic tissue and acquired immune cell subsets, such as T cell lineages. Further, as compared with naturally occurring liver tissue, liver organoids can have different efflux mechanisms, as a liver organoid can have a three-dimensional structure with a luminal structure but no ejection mechanism. In addition, liver organoids generally cannot receive dietary inputs, as they lack a gut and connected vascular channel.

Liver organoids can be derived from pluripotent stem cells (PSCs), including at least embryonic stem cells (ESCs) or induced pluripotent stem cells (iPSCs). Liver organoids may also be formed from liver-derived stem cells. In general, liver organoids can self-organize through cell sorting and spatially restricted lineage commitment in a manner similar to that which occurs in vivo, but as directed in vitro by thoughtful introduction of exogenous and/or endogenous differentiating factors and/or conditions as described herein, optionally through one or more directed steps, optionally involving introduction of one or more components.

The term “mature liver organoid” as used herein refers to liver organoids which have continued to develop from a liver organoid to include, in various embodiments, luminal projections that resemble bile canaliculi, and/or a structure having a single lumen and generally a spherical shape. Mature liver organoids may exhibit lumens with smaller sizes and reduced circularity when compared to lumens of liver organoids. In some embodiments, mature liver organoids may be generated through addition of exogenous bilirubin and/or amino acid supplementation as described herein. In some embodiments, a mature liver organoid may be characterized as expressing reduced levels of AFP, CDX2, and/or NANOG relative to liver organoids, and/or as expressing increased levels of ALB, SLC4A2 and/or HO-1 relative to liver organoids. In some embodiments, a mature liver organoid may be characterized as expressing CYP2E1, CYP7A1, PROX1, MRP3, MRP3, and/or OATP2. In some embodiments, a mature liver organoid may exhibit increased CYP3A4 and/or CYP1A2 protein levels and/or enzymatic activity relative to liver organoids.

The term “tissue culture surface” as used herein has its plain and ordinary meaning as understood in light of the specification and refers to a substrate surface on which cells may aggregate and/or adhere to facilitate cell growth, differentiation, and/or function.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. In certain embodiments, a construct and/or vector is engineered through recombinant nucleic acid technologies, and a cell is engineered through transfection or transduction of an engineered vector. Cells may be engineered to express heterologous proteins that are not naturally expressed by the cells, either because the heterologous proteins are recombinant or synthetic or because the cells do not naturally express the proteins.

### Human Liver Organoids

The microscopic architecture of the liver is made up of polygonal structures called “hepatic lobules”. Classically, these lobules take on a hexagonal structure, although other geometric shapes are observed depending on tissue specification. Each lobule unit comprises plates or layers of hepatocytes surrounding an internal central vein and encapsulated by bundles of vessels called portal triads, which are made up of a portal vein, hepatic artery, and bile duct. Hepatic activity occurs as blood flows from the portal triads at the periphery, across the hepatocytes, and into the central vein to return to the circulatory system. Due to the asymmetric organization of these lobules, the layers of hepatocytes are divided into three zones. Cells in the “periportal zone” (zone 1) are closest to the portal triad and receive the most oxygenated blood, the “pericentral zone” (zone 3) are closest to the central vein and therefore receive the least amount of oxygenated blood, and the “transition zone” (zone 2) is in between zone 1 and 3. Due to this separation, each zone of hepatocytes exhibit differing activities. For example, zone 1 hepatocytes are involved in oxidative liver functions such as gluconeogenesis and oxidative metabolism of fatty acids, whereas zone 3 hepatocytes are involved in glycolysis, lipogenesis, and cytochrome P450-mediated detoxification.

Human liver organoids (HLOs) can be derived from progenitor cells, such as, for example, patient-derived induced pluripotent stem cells (iPSCs), where the patient can be healthy or having a diseased condition, and are identical in genetic content to the respective patient. They express most liver markers that are expressed in the pre-natal stages of development. Furthermore, they are clonal and therefore reacts similarly to external stimuli and biochemical perturbations. These HLOs are highly scalable and tractable, allowing screening approaches to test a vast array of drugs and small molecules.

HLOs are easy to work with as model systems and have very low variation across batches. Large batches of HLOs can be generated within a couple of weeks. Leveraging these qualities, several drugs can be tested within a short span of time to identify pathways involved in liver diseases and disorders. In contrast, breeding model organisms such as mice and rats takes months of work and planning, and the chance of getting the desired genotype is relatively low. Furthermore, model organisms show high variations in responses to biochemical perturbations over generations. These rodents also run the risk of losing the desired genotype when bred over long periods of time, and also require complex training and procedures to model diseases and evaluate the efficacy of treatments. Compared to model organisms, genetic modifications are much easier in iPSC cell lines and they can be maintained easily over longer periods before differentiation into organoids.

Vitamin C, which is involved in the formation of the periportal zone of the liver, is synthesized by the naturally occurring enzyme L-gulonolactone oxidase (GULO). Because this enzyme is non-functional in human and some other animals such as Guinea pigs, exogenous vitamin C supplementation (typically through the diet) is necessary. As shown in Guinea pig animal models, vitamin C deficiency causes significant metabolic disorders.

Bilirubin is the naturally occurring metabolite created by normal catabolic degradation of heme. Bilirubin arises from the catalysis of biliverdin by biliverdin reductase. In the liver, bilirubin is conjugated with glucuronic acid by a family of enzymes called UDT-glucuronosyltransferases (UGTs). This conjugation renders bilirubin water soluble, enabling it to be earned in bile to the small intestine and colon, whereby it is further metabolized to waste products. Dysfunctional bilirubin metabolism, particularly due to abnormal function of UGTs preventing conjugation of bilirubin, leads to accumulation of bilirubin and is associated with various diseases characterized by hyperbilirubinemia. Notably, however, while excessive bilirubin is detrimental, bilirubin also has antioxidant capabilities and therefore may have beneficial effects in reducing oxidative damage in cells.

The enzyme L-gulonolactone oxidase (GULO) catalyzes L-gulonolactone to produce L-xylo-hex-3-gulonolactone and hydrogen peroxide. The L-xylo-hex-3-gulonolactone then spontaneously converts to ascorbate (vitamin C). Accordingly, this enzyme is involved in the biosynthesis of vitamin C, which is an essential nutrient that is involved in many biological functions such as use as a cofactor for several important enzymes and as an antioxidant. Notably, humans, as well as other haplorrhine primates, certain species of bats, and Guinea pigs have evolved to harbor a non-functional GULO gene. Therefore, these organisms are unable to synthesize ascorbate and require vitamin C intake from diet or supplementation, where a deficiency of vitamin C can lead to scurvy. As applied to the disclosure herein, a “functional GULO protein” is a GULO protein that has L-gulonolactone catalytic activity to result in the production of ascorbate. Conversely, an “inactive” GULO protein or “non-functional” GULO protein is one that does not have the catalytic activity to produce ascorbate. Humans and cells that are derived from humans comprise a non-functional GULO protein and do not have the ability to synthesize ascorbate. However, as disclosed herein, human cells may be engineered to express a functional GULO protein to enable ascorbate synthesis ability. These functional GULO proteins may be expressed in human cells (or other cells that are unable to normally synthesize ascorbate) through conventional methods of cloning, such as genetically engineering cells to have genetic sequences that encode for a functional GULO protein.

Taking advantage of this, iPSC-derived organoids expressing a functional L-gulonolactone oxidase (GULO), such as murine GULO (mGULO), have been generated. When the iPSCs and organoids are human in origin, the expression of the functional L-gulonolactone allows for ascorbate synthesis, which is normally inactive in humans. These mGULO organoids exhibit increased efficiency in conjugating bilirubin and exhibited improved viability when treated with bilirubin. The production of ascorbate in mGULO organoids reduces oxidative stress in the organoids and drives expression of NRF2, which is a master regulator of cellular detoxification pathways and in turn promotes expression of UGT1A1, which catalyzes bilirubin conjugation. These mGULO organoids are otherwise genetically identical to the patients from which they are derived, and encompass the aspects of human bilirubin metabolism. Accordingly, these organoids can be used as model systems for elucidating the mechanistic development of liver-related diseases and disorders and developing therapeutic treatments thereto.

Human Liver Organoids with Sympathetic Neurons, or Sympathetic Nerves

Previously described HLOs have been shown to exhibit hepatocytes and hepatic stellate cells. Heretofore, there has been no evidence of HLOs with sympathetic neurons, or sympathetic nerves, nor HLOs wherein a majority of the sympathetic neurons are located adjacent to the hepatic stellate cells of the liver organoid, as herein.

Accordingly, the disclosure describes the formation of a liver organoid including sympathetic neurons, or sympathetic nerves. In particular, the liver organoid includes hepatic stellate cells, hepatocytes, and sympathetic neurons, or sympathetic nerves. The sympathetic neurons, or sympathetic nerves, can form spontaneously in the appropriate conditions, when the appropriate method is performed. The sympathetic neurons, or sympathetic nerves, can perform spontaneous neural activity. The liver organoid including sympathetic neurons, or sympathetic nerves, can be a human liver organoid (HLO) and can be differentiated from pluripotent stem cells, optionally iPSCs, using differentiation methods described herein. The liver organoid including sympathetic neurons, or sympathetic nerves, can be artificial and/or three-dimensional. The liver organoid can a mature liver organoid; such as a mature human liver organoid.

In some embodiments, a majority of the sympathetic neurons are located adjacent to the hepatic stellate cells of the liver organoid. The region with sympathetic neurons can also include a sympathetic varicosity region, where a majority of the sympathetic varicosity region is localized adjacent to the hepatic stellate cells of the liver organoid. The sympathetic neurons can also include secretory vesicles, which store and secrete neurotransmitters.

The presence of the sympathetic neurons, or sympathetic nerves can be verified in various ways. For example, the sympathetic neurons can express one or more neural markers (such as, for example, class III beta tubulin (TUBB3), and the like), sympathetic markers (such as, for example, tyrosine hydroxylase (TH), and the like), membrane protein marker of secretory vesicles (such as, for example, synapsin (SYN), and the like), and/or neural crest markers (such as, for example, SOX10, FOXD3, and/or NGFR, and the like). One skilled in the art will appreciate that the liver organoids can be assayed for expression of any appropriate neural marker, sympathetic marker, membrane protein marker of secretory vesicles, and/or neural crest marker, without being limited in any way by the specific markers listed herein. The liver organoid can express neural markers, sympathetic markers, membrane protein marker of secretory vesicles, and neural crest markers, including TUBB3, TH, SYN, SOX10, FOXD3, and NGFR.

The sympathetic neurons, or sympathetic nerves, of the liver organoid can have the ability to produce one or more sympathetic nervous system neurotransmitter (such as, for example, noradrenaline, and the like) and/or one or more noradrenaline synthesizing enzymes (such as, for example, dopa decarboxylase (DDC), dopamine β-hydroxylase (DBH), and the like). The sympathetic neurons, or sympathetic nerves, of the liver organoid can also have the ability to activate β-adrenergic receptor signaling in hepatic stellate cells, e.g. via noradrenaline. The hepatic stellate cells in the liver organoid can express higher levels of β-adrenergic receptor genes (such as, for example, ADRB1, ADRB2, ADRB3, and the like) and lower levels of α2-adrenergic receptor genes (such as, for example, ADRA2A, ADRA2B, ADRA2C, and the like) than other cells. The sympathetic neurons, or sympathetic nerves, of the liver organoid can also suppress triglyceride (TG) accumulation in hepatocytes.

The liver organoid including sympathetic neurons, or sympathetic nerves, can have a structural arrangement which includes spheres of epithelial cells and aggregates of mesenchymal cells. In such arrangements, the spheres of epithelial cells can include hepatocytes, and the aggregates of mesenchymal cells can include hepatic stellate cells and sympathetic neurons; in an exemplary embodiment, the aggregates of mesenchymal cells have a higher density of hepatic stellate cells than the spheres. The cells can self-assemble into the spheres of epithelial cells including hepatocytes and aggregates of mesenchymal cells including hepatic stellate cells and sympathetic neurons, and there can be an observable and/or measurable boundary between the spheres of epithelial cells and aggregates of mesenchymal cells. In addition, the spheres of epithelial cells including hepatocytes and aggregates of mesenchymal cells including hepatic stellate cells and sympathetic neurons can self-assemble, or spontaneously self-assemble, into the liver organoid containing sympathetic neurons. The structural arrangement of the liver organoid including sympathetic neurons, or sympathetic nerves, can also include a luminal structure, such as, for example, a single lumen and/or a luminal structure having internalized microvilli.

The liver organoid including sympathetic neurons, or sympathetic nerves, can include various cell types, such as, for example, hepatoblasts, cholangiocytes, endothelial cells, macrophages, stellate cells, Schwann cells, and/or neural crest cells. The liver organoid optionally may not contain hematopoietic tissue and/or acquired immune cells.

The liver organoid including sympathetic neurons, or sympathetic nerves, can include at least 1% neural cells. For example, the liver organoid including sympathetic neurons, or sympathetic nerves, can include about 1%-75%, 2%-65%, 5%-60%, 5-40%, 5-25%, or 10-20%, neural cells. For example, the liver organoid including sympathetic neurons, or sympathetic nerves, can include at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, or greater, neural cells; for example, the liver organoid including sympathetic neurons, or sympathetic nerves, can include at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, or more, neural cells.

The liver organoid including sympathetic neurons, or sympathetic nerves, can also include about 10%-90%, 15%-75%, or 15%-65%, epithelial cells; and about 10%-90%, 15%-75%, or 15%-60%, hepatic stellate cells and hematopoietic cells. For example, the liver organoid including sympathetic neurons, or sympathetic nerves, can include at least about 10%, 20%, 25%, 30%, 35%, 40%, 45%, 50%, 55%, 60%, 65%, 70%, 75%, 80%, 85%, 90%, or greater, epithelial cells; and/or wherein the liver organoid can include at least about 10%, 20%, 25%, 30%, 35%, 40%, 45%, 50%, 55%, 60%, 65%, 70%, 75%, 80%, 85%, 90%, or greater, hepatic stellate cells and hematopoietic cells.

The liver organoid including sympathetic neurons, or sympathetic nerves, can be a useful platform for treating, modeling, and/or studying various conditions, such as, for example, fatty liver disease, MASH, MASLD, NAFLD, cirrhosis, parenteral nutrition associated liver disease (PNALD), and/or cholestasis. one or more types of liver dysfunction and/or failure, hepatitis, viral hepatitis, cholangitis, fibrosis, hepatic encephalopathy, hepatic porphyria, cirrhosis, cancer, drug-induced cholestasis, metabolic disease, autoimmune liver disease, Wilson's disease, metabolic-associated fatty liver disease, hyperammonemia, hyperbilirubinemia, Crigler-Najjar Syndrome, urea cycle disorders, Wolman disease, hepatic cancer, hepatoblastoma, metabolic dysfunction-associated steatohepatitis (MASH), metabolic dysfunction-associated liver disease (MASLD), MetALD, nonalcoholic fatty liver disease (NAFLD), drug-induced liver injury (DILI), glycogen storage disease, hemorrhagic disease, hepatic cyst, liver-related nervous system dysfunction, glucogenesis, and/or alcohol-associated liver disease. The liver organoid including sympathetic neurons, or sympathetic nerves, can be a useful platform for screening a compound or composition for treating various conditions, such as for example, fatty liver disease, MASH, MASLD, NAFLD, cirrhosis, parenteral nutrition associated liver disease (PNALD), and/or cholestasis. one or more types of liver dysfunction and/or failure, hepatitis, viral hepatitis, cholangitis, fibrosis, hepatic encephalopathy, hepatic porphyria, cirrhosis, cancer, drug-induced cholestasis, metabolic disease, autoimmune liver disease, Wilson's disease, metabolic-associated fatty liver disease, hyperammonemia, hyperbilirubinemia, Crigler-Najjar Syndrome, urea cycle disorders, Wolman disease, hepatic cancer, hepatoblastoma, metabolic dysfunction-associated steatohepatitis (MASH), metabolic dysfunction-associated liver disease (MASLD), MetALD, nonalcoholic fatty liver disease (NAFLD), drug-induced liver injury (DILI), glycogen storage disease, hemorrhagic disease, hepatic cyst, liver-related nervous system dysfunction, glucogenesis, and/or alcohol-associated liver disease.

The liver organoid including sympathetic neurons, or sympathetic nerves, can also be induced to have a fatty liver phenotype, which can render them a useful platform for treating, modeling, and/or studying various conditions involving fatty liver, such as, for example, fatty liver disease, MASH, MASLD, NAFLD, cirrhosis, parenteral nutrition associated liver disease (PNALD), and/or cholestasis. The fatty liver phenotype can be achieved by contacting the liver organoid having sympathetic neurons with one or more fatty acids (such as, for example, oleic acid, linoleic acid, palmitic acid, and the like, or any combination thereof). The fatty liver phenotype can be determined as having levels of accumulated triglycerides, such as, for example, accumulation of triglycerides in >5% of hepatocytes.

### Methods of Producing Liver Organoids

Methods of producing liver organoids have been explored previously in, for example, Ouchi et al. “Modeling Steatobepatitis in Humans with Pluripotent Stem Cell-Derived Organoids” Cell Metabolism (2019) 30 (2): 374~384; Shinozawa et al. “High-Fidelity Drug-Induced Liver Injury Screen Using Human Pluripotent Stem Cell Derived Organoids” Gastroenterology (2021) 160 (3) 831-846; PCX Publications WO 2018/085615, WO 2018/191673, WO 2018/226267, WO 2019/126626, WO 2020/023245, WO 2020/069285, WO 2020/243613, WO 2021/030373, and WO 2021/262676, each of which is hereby expressly-incorporated by references in its entirety. Disclosure of liver organoid compositions and methods of making thereof are applicable to the human liver organoids (HLOs) described herein.

Embodiments of liver organoids, liver organoids having sympathetic neurons, or sympathetic nerves, fatty liver organoids, and liver organoids having a reduced number of, or which is free of, sympathetic neurons, and compositions including the same, are provided herein. Embodiments of methods for producing liver organoids and liver organoids having sympathetic neurons, or sympathetic nerves, fatty liver organoids, and liver organoids having a reduced number of, or which is free of, sympathetic neurons, and compositions including the same, are also provided herein.

The methods of making liver organoids, as described herein and previously, generally involve one or more cellular differentiation steps, one or more of which generally involves activating or inhibiting one or more signaling pathway. One skilled in the art will appreciate that there are various ways of activating, or inhibiting a signaling pathway, in accordance with various embodiments of the methods described herein. For example, a signaling pathway can be activated by an appropriate signaling pathway activator compound or composition, or inhibited by an appropriate signaling pathway inhibitor compound or composition. It will be understood that the specific activator employed in the exemplary methods disclosed herein may have been selected out of convenience and that many options and alternatives can be available and expected to have a similar effect, provided such options and alternatives are known to those skilled in the art to have the desired effect of activating, or inhibiting, a particular signaling pathway. Further, one skilled in the art will appreciate that, as an alternative to providing a signaling pathway activator compound or composition, or signaling pathway inhibitor compound or composition, a compound or composition may be provided which results in a relevant downstream effect of signaling pathway activation, or signaling pathway inhibition, as appropriate. For example, a protein (gene product) can be provided which results from activation of a gene associated with a signaling pathway. As another example, a compound or composition can be provided which has a comparable effect to activating or inhibiting a signaling pathway, as appropriate. In this way, the effect of activating or inhibiting a signaling pathway is employed, without activating or inhibiting the signaling pathway itself. One skilled in the art will understand that it is the effect of the actual signaling pathway activation or inhibition, rather than the administration of a specific compound, which results in the desired changes within the cell culture.

Methods of Making Liver Organoids with Sympathetic Neurons, or Sympathetic Nerves

Disclosed herein are liver organoids comprising sympathetic neurons, or sympathetic nerves, as well as methods for making liver organoids comprising sympathetic neurons, or sympathetic nerves.

In some embodiments, the methods of making liver organoids comprising sympathetic neurons, or sympathetic nerves, include a) activating an FGF signaling pathway and a Wnt signaling pathway, and optionally inhibiting a BMP signaling pathway, in definitive endoderm cells (DE), for a first period of time; b) activating an FGF signaling pathway, a Wnt signaling pathway, and a retinoic acid (RA) signaling pathway, and optionally inhibiting a BMP signaling pathway, in the cells of step a), for a second period of time, thereby differentiating the DE to posterior foregut cells; c) embedding the posterior foregut cells in a basement membrane matrix, and optionally inhibiting a BMP signaling pathway in the embedded posterior foregut cells for a third period of time; and d) culturing the posterior foregut cells for a fourth period of time to differentiate the posterior foregut cells to liver organoids; wherein a BMP signaling pathway is inhibited in step b) and/or step c).

In some embodiments, the posterior foregut cells of step c) and/or step d) are cultured in a hepatocyte culture medium. In some embodiments, the hepatocyte culture medium comprises hepatocyte growth factor, oncostatin M, dexamethasone, or any combination thereof. In some embodiments, the posterior foregut cells are in the form of spheroids and/or dissociated cells.

In some embodiments of the methods of making liver organoids comprising sympathetic neurons, or sympathetic nerves, activating an FGF signaling pathway includes providing, or contacting the cells with, an FGF signaling pathway activator; activating a Wnt signaling pathway includes providing, or contacting the cells with, a Wnt signaling pathway activator; activating an RA signaling pathway includes providing, or contacting the cells with, an RA signaling pathway activator; activating a BMP signaling pathway includes providing, or contacting the cells with, a BMP signaling pathway activator; and/or inhibiting a BMP signaling pathway includes providing, or contacting the cells with, a BMP signaling pathway inhibitor.

In some embodiments, the methods comprise a) contacting definitive endoderm cells (DE) with an FGF signaling pathway activator, a Wnt signaling pathway activator, and optionally a BMP signaling pathway inhibitor for a first period of time; b) contacting the cells of step a) with the FGF signaling pathway activator, the Wnt signaling pathway activator, a retinoic acid (RA) signaling pathway activator, and optionally a BMP signaling pathway inhibitor, for a second period of time, thereby differentiating the DE to posterior foregut cells; and c) embedding the posterior foregut cells in a basement membrane matrix, and optionally contacting the embedded posterior foregut cells with a BMP signaling pathway inhibitor for a third period of time; and d) culturing the posterior foregut cells for a fourth period of time to differentiate the posterior foregut cells to liver organoids; wherein a BMP signaling pathway inhibitor is utilized in step b) and/or step c).

In some embodiments of the methods of making liver organoids comprising sympathetic neurons, or sympathetic nerves, the posterior foregut cells of steps c) and d) are cultured in a hepatocyte culture medium. In some embodiments, the hepatocyte culture medium comprises hepatocyte growth factor, oncostatin M, dexamethasone, or any combination thereof. In some embodiments, the DE has been derived from pluripotent stem cells. In some embodiments, the pluripotent stem cells are embryonic stem cells and/or induced pluripotent stem cells. In some embodiments, the posterior foregut cells are in the form of spheroids and/or dissociated cells. In some embodiments, the first period of time is, is about, is at least, is at least about, is not more than, or is not more than about, 0.5, 1, 2, 3, or 4 days. In some embodiments, the second period of time is, is about, is at least, is at least about, is not more than, or is not more than about 0.5, 1, 2, or 3 days. In some embodiments, the third period of time is, is about, is at least, is at least about, is not more than, or is not more than about, 0.5, 1, 2, 3, 4, 5, 6, or 7 days. In some embodiments, the fourth period of time is, is about, is at least, is at least about, is not more than, or is not more than about, 4, 5, 6, 7, 8, 9, 10, 11, 12, 13, 14, 15, 16, 17, 18, 19, 20, 21, 22, 23, 24, 25, 26, 27, 28, 29, or 30 days, optionally at least 4 days. In some embodiments, the posterior foregut spheroids are embedded in the basement membrane matrix at a concentration that is, is about, is at least, is at least about, is not more than, or is not more than about, 5×10⁴, 6×10⁴, 7×10⁴, 8×10⁴, 9×10⁴, 1×10⁵, 2×10⁵, 3×10⁵, 4×10⁵, 5×10⁵, 6.0×10⁵ cells, 7.0×10⁵ cells, 8.0×10⁵ cells, 9.0×10⁵ cells, 10.0×10⁵ cells, or higher, cells per μL of basement membrane matrix, or any concentration of cells per μL within a range defined by any two of the aforementioned concentrations, for example, 5×10⁴-5×10⁵, 5×10⁴-1×10⁵, 1×10⁵-5×10⁵, 6×10⁴-4×10⁵, 8×10⁴-3×10⁵ cells, or 1×10⁵-10×10⁵, per μL of basement membrane matrix. In some embodiments, the posterior foregut cells are seeded in a basement membrane matrix at a seeding density of at least about 1.0×10⁵, or at least about 5.0×10⁵ cells per μL of basement membrane matrix, In some embodiments, the basement membrane matrix is Matrigel. In some embodiments, the liver organoid, DE, and/or pluripotent stem cells are derived from a patient. In some embodiments, the liver organoid, DE, and/or pluripotent stem cells are derived from a patient having a liver disease. In some embodiments, the liver organoid, DE, and/or pluripotent stem cells are derived from a patient having fatty liver disease.

In some embodiments of the methods of making liver organoids comprising sympathetic neurons, or sympathetic nerves, wherein activating the FGF signaling pathway activator includes providing an FGF signaling pathway activator, the FGF signaling pathway activator can be selected from the group consisting of FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF19, FGF20, FGF21, FGF22, and FGF23. In some embodiments, the FGF signaling pathway activator is FGF4. In some embodiments, the FGF signaling pathway activator is provided at a concentration that is, is about, is at least, is at least about, is not more than, or is not more than about, 100, 150, 200, 250, 300, 350, 400, 450, 500, 550, 600, 650, 700, 750, 800, 850, 900, 950, or 1000 ng/ml, or any concentration within a range defined by any two of the aforementioned concentrations, including 100-1000 ng/mL, 100-500 ng/ml, 500-1000 ng/mL, 250-750 ng/ml, or 400-600 ng/mL. In some embodiments, the FGF signaling pathway activator is provided at a concentration of 500 ng/mL or about 500 ng/mL.

In some embodiments of the methods of making liver organoids comprising sympathetic neurons, or sympathetic nerves, wherein activating the Wnt signaling pathway activator includes providing a Wnt signaling pathway activator, the Wnt signaling pathway activator can be selected from the group consisting of Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, CHIR99021, CHIR 98014, AZD2858, BIO, AR-A014418, SB 216763, SB 415286, aloisine, indirubin, alsterpaullone, kenpaullone, lithium chloride, TDZD 8, and TWS119. In some embodiments, the Wnt signaling pathway activator is CHIR99021. In some embodiments, the Wnt signaling pathway activator is provided at a concentration that is, is about, is at least, is at least about, is not more than, or is not more than about, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, or 3.5 μM, or any concentration within a range defined by any two of the aforementioned concentrations, including 0.5-3.5 M, 0.5-2 μM, 2-3.5 μM, 1-3 μM, or 1.5-2.5 μM. In some embodiments, the Wnt signaling pathway activator is provided at a concentration of 2 μM or about 2 μM. In some embodiments, the Wnt signaling pathway activator is provided at a concentration of no more than 2 μM or about 2 μM. In some embodiments, the Wnt signaling pathway activator is provided at a concentration of no more than 3 μM or about 3 μM.

In some embodiments of the methods of making liver organoids comprising sympathetic neurons, or sympathetic nerves, wherein activating the BMP signaling pathway activator includes providing a BMP signaling pathway activator, the BMP signaling pathway inhibitor can be selected from the group consisting of Noggin, RepSox, LY364947, LDN-193189, and SB431542. In some embodiments, the BMP signaling pathway inhibitor is LDN-193189 and/or SB431542. In some embodiments, the BMP signaling pathway inhibitor, optionally LDN-193189 and/or SB431542, is provided at a concentration that is, is about, is at least, is at least about, is not more than, or is not more than about, 50, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 450, 500, 550, 600, 650, 700, 750, 800, 850, 900, 950, 1000, 1050, 1100, 1150, 1200, 1250, 1300, 1350, 1400, 1450, 1500 nM, or any concentration within a range defined by any two of the aforementioned concentrations, including 50-1500 nM, 100-1100 nM, 100-700 nM, 200-600 nM, 150-350 nM, 200-300 nM, 100-1250 nM, 250-1250 nM, 250-1000 nM, 250-750 nM, 400-600 nM, 500-1250 nM, 750-1250 nM, or 900-1100 nM; optionally at a concentration of 250 nM or about 250 nM, at a concentration of 500 nM or about 500 nM, at a concentration of 750 nM or about 750 nM, or at a concentration of 1000 nM or about 1000 nM. In some embodiments, the BMP signaling pathway inhibitor, optionally LDN-193189, is provided at a concentration of 250 nM or about 250 nM. In some embodiments, the BMP signaling pathway inhibitor, optionally LDN-193189 and/or SB431542, is provided at a concentration of 500 nM or about 500 nM. In some embodiments, the BMP signaling pathway inhibitor, optionally LDN-193189 and/or SB431542, is provided at a concentration of 1000 nM or about 1000 nM. In some embodiments, the BMP signaling pathway inhibitor, optionally LDN-193189 and/or SB431542, is contacted for 3 days or about 3 days during the first period of time on about day 3 of differentiation induction of the liver organoid. In some embodiments, the BMP signaling pathway inhibitor, optionally LDN-193189 and/or SB431542, is provided at a concentration of 250 nM or about 250 nM. In some embodiments, the BMP signaling pathway inhibitor, optionally LDN-193189 and/or SB431542, is contacted at a concentration of 500 nM or about 500 nM. In some embodiments, the BMP signaling pathway inhibitor, optionally LDN-193189 and/or SB431542, is provided at a concentration of 1000 nM or about 1000 nM.

In some embodiments of the methods of making liver organoids comprising sympathetic neurons, or sympathetic nerves, wherein activating the RA signaling pathway activator includes providing a RA signaling pathway activator, the RA signaling pathway activator can be selected from the group consisting of retinoic acid, all-trans retinoic acid, 9-cis retinoic acid, CD437, EC23, BS 493, TTNPB, and AM580. In some embodiments, the RA signaling pathway activator is RA. In some embodiments, the RA signaling pathway activator is contacted at a concentration that is, is about, is at least, is at least about, is not more than, or is not more than about, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.9, or 3 μM, or any concentration within a range defined by any two of the aforementioned concentrations, including 1-3 μM, 1-2 μM, 2-3 μM, or 1.5-2.5 μM. In some embodiments, the RA signaling pathway activator is provided at a concentration of 2 μM or about 2 μM.

In some embodiments, the first period of time is, is about, is at least, is at least about, is not more than, or is not more than about, 0.5, 1, 2, 3, or 4 days; optionally at least about 1, 2, or 3 days. In some embodiments, the first period of time is, is about, is at least, is at least about, is not more than, or is not more than about, 2 days. In some embodiments, the second period of time is, is about, is at least, is at least about, is not more than, or is not more than about, 0.5, 1, 2, or 3 days; optionally at least about 0.5, 1, or 2 days. In some embodiments, the second period of time is, is about, is at least, is at least about, is not more than, or is not more than about, 1 day. In some embodiments, the third period of time is, is about, is at least, is at least about, is not more than, or is not more than about, 0.5, 1, 2, 3, 4, 5, 6, or 7 days; optionally at least about 1, 2, 3, 4, 5, or 6 days. In some embodiments, the fourth period of time is, is about, is at least, is at least about, is not more than, or is not more than about, 10, 11, 12, 13, 14, 15, 16, 17, 18, 19, 20, 21, 22, 23, 24, 25, 26, 27, 28, 29, or 30 days, or at least 10 days.

In some embodiments, the posterior foregut spheroids are embedded in the basement membrane matrix at a concentration that is, is about, is at least, is at least about, is not more than, or is not more than about, 6×10⁴, 7×10⁴, 8×10⁴, 9×10⁴, 1×10⁵, 2×10⁵, 3×10⁵, 4×10⁵, 5.0×10⁵ cells, 6.0×10⁵ cells, 7.0×10⁵ cells, 8.0×10⁵ cells, 9.0×10⁵ cells, 10.0×10⁵ cells, or higher, per μL of basement membrane matrix, or any concentration of cells per μL within a range defined by any two of the aforementioned concentrations, for example, about 6×10⁴-4×10⁵, 6×10⁴-1×10⁵, 1×10⁵-4×10⁵, or 8×10⁴-3×10⁵, 1.0×10⁵-10.0×10⁵ cells per μL of basement membrane matrix. In some embodiments, the posterior foregut spheroids are embedded in the basement membrane matrix at a concentration that is at least about 1.0×10⁵, or at least about 5.0×10⁵ cells per μL of basement membrane matrix. In some embodiments, the posterior foregut spheroids are embedded in the basement membrane matrix at a concentration that is, is about, is at least, is at least about, is not more than, or is not more than about 8×10⁴, 9×10⁴, 1×10⁵, 2×10⁵, 3×10⁵ cells per μL of basement membrane matrix, or any concentration of cells per μL within a range defined by any two of the aforementioned concentrations, for example, 8×10⁴-3×10⁵, 8×10⁴-1×10⁵, 1×10⁵-3×10⁵, or 9×10⁴-2×10⁵ cells per μL of basement membrane matrix. In some embodiments, the posterior foregut spheroids are embedded in the basement membrane matrix at a concentration that is at least about 1×10⁵ cells per μL of basement membrane matrix.

In some embodiments, the FGF signaling pathway activator is FGF4. In some embodiments, the FGF signaling pathway activator is provided at a concentration that is, is about, is at least, is at least about, is not more than, or is not more than about, 250, 300, 350, 400, 450, 500, 550, 600, 650, 700, or 750 ng/mL, or any concentration within a range defined by any two of the aforementioned concentrations, including 250-750 ng/mL, 250-500 ng/ml, 500-750 ng/ml, or 400-600 ng/mL. In some embodiments, the Wnt signaling pathway activator is CHIR99021. In some embodiments, the Wnt signaling pathway activator is provided at a concentration that is, is about, is at least, is at least about, is not more than, or is not more than about, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 μM, or any concentration within a range defined by any two of the aforementioned concentrations, including 1.0-3.0 μM, 1.0-2 μM, 2-3.0 μM, or 1.5-2.5 μM. In some embodiments, the Wnt signaling pathway activator is provided at a concentration of no more than 2 μM or about 2 μM. In some embodiments, the Wnt signaling pathway activator is provided at a concentration of no more than 3 μM or about 3 μM. In some embodiments, the BMP signaling pathway inhibitor is LDN-193189. In some embodiments, the BMP signaling pathway inhibitor, optionally LDN-193189 and/or SB431542, is provided at a concentration that is, is about, is at least, is at least about, is not more than, or is not more than about, 150, 160, 170, 180, 190, 200, 210, 220, 230, 240, 250, 260, 270, 280, 290, 300, 310, 320, 330, 340, 350, 400, 450, 500, 550, 600, 650, 700, 750, 800, 850, 900, 950, 1000, 1050, 1100, 1150, 1200, or 1250 nM, or any concentration within a range defined by any two of the aforementioned concentrations, including 150-350 nM, 150-250 nM, 250-350 nM, 200-300 nM, 250-1250 nM, 250-1000 nM, 250-750 nM, 400-600 nM, 500-1250 nM, 750-1250 nM, or 900-1100 nM. In some embodiments, the BMP signaling pathway inhibitor, optionally LDN-193189 and/or SB431542, is provided at a concentration of 250 nM or about 250 nM. In some embodiments, the BMP signaling pathway inhibitor, optionally LDN-193189 and/or SB431542, is provided at a concentration of 500 nM or about 500 nM. In some embodiments, the BMP signaling pathway inhibitor, optionally LDN-193189 and/or SB431542, is contacted at a concentration of 1000 nM or about 1000 nM. In some embodiments, the BMP signaling pathway inhibitor, optionally LDN-193189 and/or SB431542, is provided for at least 2 days or about 3 days during the second period of time on about day 3 of differentiation induction of the liver organoid. In some embodiments, the BMP signaling pathway inhibitor, optionally LDN-193189 and/or SB431542, is provided for at least 2 days or about 3 days, 4 days, 5 days, 6 days, or longer during the third period of time with the embedded posterior foregut cells. In some embodiments, the RA signaling pathway activator is retinoic acid. In some embodiments, the RA signaling pathway activator is provided at a concentration that is, is about, is at least, is at least about, is not more than, or is not more than about, 1.5, 1.6, 1.7, 1.8, 1.9, 2, 2.1, 2.2, 2.3, 2.4, or 2.5 M, or any concentration within a range defined by any two of the aforementioned concentrations, including 1.5-2.5 μM, 1.5-2 μM, 2-2.5 μM, or 1.5-2.5 μM.

In some embodiments, the FGF signaling pathway activator is FGF4. In some embodiments, the FGF signaling pathway activator is provided at a concentration that is, is about, is at least, is at least about, is not more than, or is not more than about, 500 ng/ml. In some embodiments, the Wnt signaling pathway activator is CHIR99021. In some embodiments, the Wnt signaling pathway activator is provided at a concentration that is, is about, is at least, is at least about, is not more than, or is not more than about, 2.0 μM. In some embodiments, the BMP signaling pathway inhibitor is LDN-193189 and/or SB431542. In some embodiments, the BMP signaling pathway inhibitor, optionally LDN-193189 and/or xxx, is provided at a concentration that is, is about, is at least, is at least about, is not more than, or is not more than about, 250 nM. In some embodiments, the BMP signaling pathway inhibitor, optionally LDN-193189 and/or SB431542, is provided at a concentration that is, is about, is at least, is at least about, is not more than, or is not more than about, 500 nM. In some embodiments, the BMP signaling pathway inhibitor, optionally LDN-193189 and/or SB431542, is provided at a concentration that is, is about, is at least, is at least about, is not more than, or is not more than about, 1000 nM. In some embodiments, the BMP signaling pathway inhibitor, optionally LDN-193189 and/or SB431542, is provided for 3 days or about 3 days during the first period of time on about day 3 of differentiation induction of the liver organoid. In some embodiments, the RA signaling pathway activator is retinoic acid. In some embodiments, the RA signaling pathway activator is provided at a concentration that is, is about, is at least, is at least about, is not more than, or is not more than about, 2 μM.

The methods for making liver organoids provided herein can produce liver organoids comprising sympathetic neurons, or sympathetic nerves, which can be used to model, study, treat, and/or screen compounds for treatment of liver-related disease and disorders, as described herein. The liver is naturally innervated by the sympathetic and parasympathetic nervous systems. These nervous systems regulate important functions and properties of the liver, such as regulating metabolism, which may involve the production and secretion of bile, and response to glucose and lipid levels in the blood. Liver nervous system dysfunction may play a role in diseases such as obesity, dyslipidemia, hypertension, and diabetes. Liver nervous system dysfunction can also be involved in conditions including liver dysfunction and/or failure, hepatitis, viral hepatitis, cholangitis, fibrosis, hepatic encephalopathy, hepatic porphyria, cirrhosis, cancer, drug-induced cholestasis, metabolic disease, autoimmune liver disease, Wilson's disease, metabolic-associated fatty liver disease, hyperammonemia, hyperbilirubinemia, Crigler-Najjar Syndrome, urea cycle disorders, Wolman disease, hepatic cancer, hepatoblastoma, metabolic dysfunction-associated steatohepatitis (MASH), metabolic dysfunction-associated liver disease (MASLD), MetALD, nonalcoholic fatty liver disease (NAFLD), drug-induced liver injury (DILI), glycogen storage disease, hemorrhagic disease, hepatic cyst, liver-related nervous system dysfunction, and/or alcohol-associated liver disease. Hepatic sympathetic innervation can also be involved in conditions such as gluconeogenesis, and liver organoids comprising sympathetic neurons, or sympathetic nerves, can be used to model, study, treat, and/or screen compounds for treatment of such conditions as well.

In particular, the liver nervous system can be especially relevant in metabolic dysfunction-steatotic liver disease (MASLD), which is a new disease concept designed to efficiently identify high-risk patients, elucidate the pathophysiology and develop treatment methods. MASLD is diagnosed when fatty liver is coexisting with either obesity, type 2 diabetes, or two or more metabolic disorders. As generally understood in the art and demonstrated herein, sympathetic neurons in liver tissue and the liver organoids provided herein have the ability to secrete neurotransmitters such as noradrenaline (which affect hepatic cells such as stellate cells, which express adrenergic receptors including α1, α2, and β adrenergic receptors).

In some embodiments, the sympathetic neurons found in the liver organoids disclosed herein exhibit class III beta tubulin (TUBB3)-positive varicosities, express tyrosine hydroxylase (TH), and secrete synapsin (SYN)-positive vesicles. In some embodiments, the ability for the liver organoids disclosed herein to produce noradrenaline is evidenced by expression of dopa decarboxylase (DDC) and dopamine β-hydroxylase (DBH). In some embodiments, the liver organoids disclosed herein also comprise hepatic stellate cells and hepatocytes, and the sympathetic neurons of the liver organoid generally localizes with the vimentin (VIM)-positive stellate cells. In some embodiments, at least 55%, 60%, 65%, 70%, 75%, 80%, 85%, or 90% of the sympathetic neurons are located adjacent to the hepatic stellate cells.

Some embodiments of the present disclosure relate to the liver organoids comprising sympathetic neurons produced according to the methods described herein or otherwise known in the art. In some embodiments, these liver organoids can be generated from pluripotent stem cells isolated from a patient. In some embodiments, the patient may have a liver disease, was previously treated for a liver disease, and/or is predisposed to contracting a liver disease.

In some embodiments, the liver organoids having sympathetic neurons, or sympathetic nerves, disclosed herein (including liver organoids comprising sympathetic neurons) can be induced to have a fatty liver phenotype. In some embodiments, the liver organoids having sympathetic neurons, or sympathetic nerves, may be induced to have a fatty liver phenotype by contacting the liver organoid with fatty acids, such as oleic acid, linoleic acid, palmitic acid, or any combination thereof. Methods of making a fatty liver organoid may be found in PCT Publication WO 2018/085622, hereby expressly incorporated by reference in its entirety.

Liver Organoids with Reduced or No Sympathetic Neurons

Also disclosed herein are methods for making liver organoids with a reduced number of, or free of, sympathetic neurons. In some embodiments, the methods comprise inhibiting a pathway associated with sympathetic neuron survival in a liver organoid. In some embodiments, inhibiting a pathway associated with sympathetic neuron survival in a liver organoid includes providing an inhibitor of sympathetic neuron survival. In some embodiments, the methods comprise contacting a liver organoid with an inhibitor of sympathetic neuron survival. In some embodiments, the inhibition, or contacting, ablates the sympathetic neurons from the liver organoid, thereby making a liver organoid having a reduced number of, or which is free of, sympathetic neurons. In some embodiments, the inhibitor of sympathetic neuron survival is a neurotoxin. In some embodiments, the inhibitor of sympathetic neuron survival is selected from the group consisting of 6-hydroxydopamine (6-OHDA), DSP-4, MPP+, or MPTP, or any combination thereof. In some embodiments, the inhibitor of sympathetic neuron survival is 6-OHDA. In some embodiments, the liver organoid provided with the inhibitor of sympathetic neuron survival is any one of the liver organoids comprising sympathetic neurons produced by the methods disclosed herein.

Also disclosed herein in some embodiments are liver organoids having a reduced number of, or which is free of, sympathetic neurons. In some embodiments, the liver organoid having a reduced number of, or which is free of, sympathetic neurons has been treated with an inhibitor of sympathetic neuron survival. In some embodiments, the inhibitor of sympathetic neuron survival is a neurotoxin. In some embodiments, the inhibitor of sympathetic neuron survival is selected from the group consisting of 6-OHDA, DSP-4, MPP+, or MPTP, or any combination thereof. In some embodiments, the inhibitor of sympathetic neuron survival is 6-OHDA.

By using the methods of inhibiting sympathetic neuron survival, and/or ablating the sympathetic neurons from a human liver organoid, such as a human liver organoid comprising sympathetic neurons, or sympathetic nerves, sympathetic nerve activity can be artificially regulated in a human liver organoid. The ability to remove, ablate, or inhibit the formation of neural cells allows for the ability to construct liver organoids with a bespoke amount of neural cells and to artificially regulate neural activity. This type of model leads to the identification of sympathetic effects on human liver development and the elucidation of their mechanisms of action.

### Fatty Liver Organoids

In some embodiments, the liver organoids disclosed herein (including liver organoids comprising sympathetic neurons and liver organoids having a reduced number of, or which is free of, sympathetic neurons) can be induced to have a fatty liver phenotype. In some embodiments, the liver organoids may be induced to have a fatty liver phenotype by providing the liver organoid with fatty acids, such as oleic acid, linoleic acid, palmitic acid, or any combination thereof. Methods of making a fatty liver organoid may be found in PCT Publication WO 2018/085622, hereby expressly incorporated by reference in its entirety.

Disclosed herein are methods of making a fatty liver organoid having a reduced number of, of which is free of, sympathetic neurons and comprising a fatty liver phenotype. In some embodiments, the methods comprise providing the liver organoid having a reduced number of, or which is free of, sympathetic neurons with one or more fatty acids, thereby inducing the fatty liver phenotype in the liver organoid having a reduced number of, or which is free of, sympathetic neurons. In some embodiments, the one or more fatty acids comprise oleic acid, linoleic acid, palmitic acid, or any combination thereof. In some embodiments, the liver organoid having a reduced number of, or which is free of, sympathetic neurons is the liver organoid having a reduced number of, or which is free of, sympathetic neurons as made by any of the methods disclosed herein.

Also provided herein is the fatty liver organoid having a reduced number of, of which is free of, sympathetic neurons made by any of the methods disclosed herein.

Also disclosed herein are methods of making a fatty liver organoid comprising a fatty liver phenotype. In some embodiments, the methods comprise inhibiting sympathetic neuron survival in a liver organoid, in combination with, or concurrently, consecutively, or non-consecutively, providing the liver organoid with one or more fatty acids, thereby inducing the fatty liver phenotype in the liver organoid. In some embodiments, the methods comprise providing a liver organoid with an inhibitor of sympathetic neuron survival and one or more fatty acids, thereby inducing the fatty liver phenotype in the liver organoid. In some embodiments, the inhibitor of sympathetic neuron survival is a neurotoxin. In some embodiments, the inhibitor of sympathetic neuron survival is selected from the group consisting of 6-hydroxydopamine (6-OHDA), DSP-4, MPP+, or MPTP, or any combination thereof. In some embodiments, the inhibitor of sympathetic neuron survival is 6-OHDA. In some embodiments, the one or more fatty acids comprise oleic acid, linoleic acid, palmitic acid, or any combination thereof. In some embodiments, the liver organoid may be any of the liver organoids disclosed herein or otherwise known in the art (including liver organoids comprising sympathetic neurons).

Also provided herein is the fatty liver organoid made by any of the methods disclosed herein.

Methods of Using Liver Organoids, Including Those with Reduced or No Sympathetic Neurons

Provided herein are methods of treating a liver-related disease or disorder, the methods including transplanting, into a subject having a liver-related disease or disorder, the liver organoid comprising sympathetic neurons, the artificial liver organoid comprising sympathetic neurons, the liver organoid having a reduced number, or absent of, sympathetic neurons, the fatty liver organoid, and/or a cell composition, and/or an ex vivo composition including the same. Such methods can be useful in treating a liver-related disease or disorder, such as, for example, one or more types of liver dysfunction and/or failure, hepatitis, viral hepatitis, cholangitis, fibrosis, hepatic encephalopathy, hepatic porphyria, cirrhosis, cancer, drug-induced cholestasis, metabolic disease, autoimmune liver disease, Wilson's disease, metabolic-associated fatty liver disease, hyperammonemia, hyperbilirubinemia, Crigler-Najjar Syndrome, urea cycle disorders, Wolman disease, hepatic cancer, hepatoblastoma, metabolic dysfunction-associated steatohepatitis (MASH), metabolic dysfunction-associated liver disease (MASLD), MetALD, nonalcoholic fatty liver disease (NAFLD), drug-induced liver injury (DILI), glycogen storage disease, hemorrhagic disease, hepatic cyst, liver-related nervous system dysfunction, glucogenesis, and/or alcohol-associated liver disease. The liver organoid comprising sympathetic neurons can be particularly useful in treating liver-related nervous system dysfunctions, such as, for example, MASLD, obesity, dyslipidemia, hypertension, and/or diabetes. Following transplantation, the subject can have improved symptoms of biliary stricture and/or liver regeneration, and/or an increased survival rate following transplantation. In some embodiments of the treatment, the transplanted liver organoids can engraft onto the liver of the subject.

Also provided herein are methods of using the liver organoid comprising sympathetic neurons, the artificial liver organoid comprising sympathetic neurons, the liver organoid having a reduced number, or absent of, sympathetic neurons, the fatty liver organoid, and/or a cell composition, and/or an ex vivo composition including the same, as an in vitro human model system for studying hepatocyte function and developmental divergence; studying liver-related disease; identifying therapeutic targets; and/or identifying therapeutic compounds and/or compositions effective in treating a liver-related disease or disorder.

Also provided herein are methods of modulating sympathetic neuron activity, function, or survival in a liver organoid. In some embodiments, the methods comprise activating or inhibiting of sympathetic neuron function and/or sympathetic neuron survival in a liver organoid. In some embodiments, the methods comprise providing a liver organoid with an activator or inhibitor of sympathetic neuron function and/or an activator or inhibitor of sympathetic neuron survival. In some embodiments, the activator or inhibitor of sympathetic neuron function modulate noradrenaline production ability of the liver organoid. In some embodiments, the activator or inhibitor of sympathetic neuron function is an activator or inhibitor of adrenergic receptor function of sympathetic neurons. In some embodiments, the activator of sympathetic neuron function comprises noradrenaline, isoproterenol, phenylephrine, or any combination thereof. In some embodiments, the inhibitor of sympathetic neuron function comprises prazosin, propranolol, venlafaxine, levomilnacipran, or any combination thereof. In some embodiments, the inhibitor of sympathetic neuron function or survival suppresses sympathetic neuron production and/or the elongation of sympathetic neurons. In some embodiments, the inhibitor of sympathetic neuron function or survival is a BDNF inhibitor, including a BDNF pathway inhibitor. In some embodiments, the BDNF inhibitor is licochalcone A, K252a, GZD2202, cyclotraxin B, ANA12 or any combination thereof. In some embodiments, the BDNF inhibitor is ANA12. In some embodiments, the BDNF inhibitor, optionally ANA12, is provided at a concentration that is, is about, is at least, is at least about, is not more than, or is not more than about, 1 μM. In some embodiments, the BDNF inhibitor is provided at a concentration that is, is about, is at least, is at least about, is not more than, or is not more than about, 0.1, 0.2, 0.3, 0.4, 0.5, 0.6, 0.7, 0.8, 0.9, 1, 1.1, 1.2, 1.3, 1.4 1.5, 1.6, 1.7, 1.8, 1.9, 2, 2.1, 2.2, 2.3, 2.4, or 2.5 μM, or any concentration within a range defined by any two of the aforementioned concentrations, including 0.2-2.5 μM, 0.5-1.5 μM, 0.8-1.2 μM, or 0.9-1.1 μM. In some embodiments, the BDNF inhibitor, optionally ANA12, is provided at a concentration of 1 μM or about 1 μM for 5 days or about 5 days on about day 15 of differentiation induction of the liver organoid. In some embodiments, the activator of sympathetic neuron survival induces sympathetic neuron production and/or the elongation of sympathetic neurons. In some embodiments, the activator of sympathetic neuron survival comprises nerve growth factor (NGF), glial cell like-derived neurotrophic factor (GDNF), brain derived neurotrophic factor (BDNF), ciliary neurotrophic factor (CNTF), netrin 1 (NTN1), anti-tumor necrosis factor alpha (TNF), or any combination thereof. In some embodiments, the activator of sympathetic neuron survival is BDNF. In some embodiments, the activator of sympathetic neuron survival, optionally BDNF, is provided at a concentration that is, is about, is at least, is at least about, is not more than, or is not more than about, 50 nM, 60 nM, 70 nM, 80 nM, 90 nM, 100 nM, 110 nM, 120 nM, 130 nM, 140 nM, or 150 nM, or any concentration within a range defined by any two of the aforementioned concentrations, including 50-150 nM, 75-125 nM, 90-110 nM, or 95-105 nM. In some embodiments, the activator of sympathetic neuron survival, optionally BDNF, is provided at a concentration of 100 nM or about 100 nM for 13 days or about 13 days beginning on, or on about, day 15, optionally day 14-16, of differentiation induction of the liver organoid. In some embodiments, the inhibitor of sympathetic neuron function comprises a neurotoxin. In some embodiments, the inhibitor of sympathetic neuron survival is a neurotoxin. In some embodiments, the inhibitor of sympathetic neuron survival comprises 6-hydroxydopamine (6-OHDA), DSP-4, MPP+, or MPTP, or any combination thereof. In some embodiments, the activator or inhibitor of sympathetic neuron function and/or the activator or inhibitor of sympathetic neuron survival comprises an interfering RNA (RNAi) or site-directed endonuclease. In some embodiments, the site-directed endonuclease is CRISPR/Cas9. In some embodiments, the RNAi or site-directed endonuclease targets one or more proteins and/or genes involved in noradrenaline production. In some embodiments, the RNAi or site-directed endonuclease modulates expression of the one or more proteins and/or genes involved in noradrenaline production. In some embodiments, the methods further comprise observing the effects of the activator or inhibitor of sympathetic neuron function and/or the activator or inhibitor of sympathetic neuron survival on the liver organoid. For example, in some embodiments, the methods further comprise observing a change in triglycerides in the liver organoid in response to contacting the activator or inhibitor of sympathetic neuron function and/or the activator or inhibitor of sympathetic neuron survival. In some embodiments, the change in triglycerides in the liver organoid may be a decrease in the levels of triglycerides in the liver organoid. In some embodiments, the change in triglycerides in the liver organoid may be an increase in the levels of triglycerides in the liver organoid. In some embodiments, the liver organoid is a fatty liver organoid comprising a fatty liver phenotype. In some embodiments, the liver organoid is any one of the liver organoids disclosed herein or otherwise known in the art.

Also disclosed herein are methods of screening for compounds or compositions that affect fatty liver phenotype, including those useful in the treatment of fatty liver disease. The methods comprise contacting a liver organoid comprising a fatty liver phenotype (including those liver organoids disclosed herein, e.g. those induced to have a fatty liver phenotype and which may have sympathetic neurons, or which may or may not have reduced or no sympathetic neurons) with a compound or composition and detecting a change in the fatty liver phenotype of the liver organoid. The methods comprise contacting a liver organoid having sympathetic neurons, or sympathetic nerves, and comprising a fatty liver phenotype with a compound or composition and detecting a change in the fatty liver phenotype of the liver organoid. The methods comprise contacting a liver organoid having a reduced number or absent of sympathetic neurons, or sympathetic nerves, and comprising a fatty liver phenotype with a compound or composition and detecting a change in the fatty liver phenotype of the liver organoid. In some embodiments, detecting the change in the fatty liver phenotype comprises detecting a change in triglycerides in the fatty liver organoid after contacting with the compound or composition. In some embodiments, detecting the change in triglycerides comprises detecting a reduction in triglycerides in the fatty liver organoid, thereby resulting in an improvement in the fatty liver phenotype of the fatty liver organoid. In some embodiments, detecting the change in triglycerides comprises detecting an increase in triglycerides in the fatty liver organoid, thereby resulting in a worsening in the fatty liver phenotype of the fatty liver organoid. In some embodiments, the compounds or compositions that activator or promote sympathetic neuron activity or survival may result in a decrease in triglycerides in the fatty liver organoid. In some embodiments, the compounds or compositions that inhibit sympathetic neuron activity or survival may result in an increase in triglycerides in the fatty liver organoid. In some embodiments, detecting a reduction in triglycerides is with a lipophilic fluorescent probe. In some embodiments, the lipophilic fluorescent probe is a BODIPY probe, BODIPY 493/503, BODIPY 558/568 C 12, or Oil red O. In some embodiments, the lipophilic fluorescent probe is BODIPY 493/503. In some embodiments, wherein the reduction in triglycerides in the liver organoid is at least 5%, 10%, 15%, 20%, 25%, 30%, 35%, 40%, 45%, or 50% relative to the level of triglycerides in the liver organoid before contacting with the compound or composition. In some embodiments, the fatty liver organoid is any one of the fatty liver organoids disclosed herein.

Also disclosed herein are methods of screening for compounds or compositions that can have efficacy in treating a liver-related disease or disorder, including those useful in the treatment of fatty liver disease. The methods comprise contacting a liver organoid as disclosed herein, e.g. human liver organoids which may have sympathetic neurons, or which may or may not have reduced or no sympathetic neurons, with a compound or composition and detecting a change in the liver organoid.

### Stem Cells

The term “totipotent stem cells” (also known as omnipotent stem cells) as used herein has its plain and ordinary meaning as understood in light of the specification and are stem cells that can differentiate into embryonic and extra-embryonic cell types. Such cells can construct a complete, viable organism. These cells are produced from the fusion of an egg and sperm cell. Cells produced by the first few divisions of the fertilized egg are also totipotent.

The term “embryonic stem cells (ESCs),” also commonly abbreviated as ES cells, as used herein has its plain and ordinary meaning as understood in light of the specification and refers to cells that are pluripotent and derived from the inner cell mass of the blastocyst, an early-stage embryo. For purpose of the present disclosure, the term “ESCs” is used broadly sometimes to encompass the embryonic germ cells as well.

The term “pluripotent stem cells (PSCs)” as used herein has its plain and ordinary′ meaning as understood in light of the specification and encompasses any cells that can differentiate into nearly all cell types of the body, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system), PSCs can be the descendants of inner cell mass cells of the preimplantation blastocyst or obtained through induction of a non-pluripotent cell, such as an adult somatic cell, by forcing the expression of certain genes. Pluripotent stem cells can be derived from any suitable source. Examples of sources of pluripotent stem cells include mammalian sources, including human, rodent, porcine, and bovine.

The term “induced pluripotent stem cells (iPSCs),” also commonly abbreviated as iPS cells, as used herein has its plain and ordinary meaning as understood in light of the specification and refers to a type of pluripotent stem cells artificially derived from a normally non-pluripotent cell, such as an adult somatic cell, by inducing a “forced” expression of certain genes. hiPSC refers to human iPSCs. In some methods known in the art, iPSCs may be derived by transfection of certain stem cell-associated genes into non-pluripotent cells, such as adult fibroblasts. Transfection may be achieved through viral transduction using viruses such as retroviruses or lentiviruses. Transfected genes may include the master transcriptional regulators Oct-3/4 (PUU5F1) and Sox2, although other genes may enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include first generation iPSCs, second generation iPSCs in mice, and human induced pluripotent stem cells. In some methods, a retroviral system is used to transform human fibroblasts into pluripotent stem cells using four pivotal genes: Oct3/4, Sox2, Klf4, and c-Myc. In other methods, a lentiviral system is used to transform somatic cells with GCT4, SOX2, NANOG, and LIN28. Genes whose expression are induced in iPSCs include but are not limited to Oct-3/4 (POU5F1); certain members of the Sox gene family (e.g., Sox1, Sox2, Sox3, and Sox15); certain members of the Klf family (e.g., Klf1, Klf2, Klf4, and Klf5), certain members of the Mye family (e.g., C-myc, L-myc, and N-myc), Nanog, LIN28, Tert, Fbx15, ERas, EC ATI 5-1, ECAT15-2, Tell, b-Catenm, EC ATI, Esgi, Dnmt3L, EC ATS, Gdf3, Fthll7, Sall4, Rexl, UTF1, Stella, Stat3, Grb2, Prdml4, Nr5a1, Nr5a2, or E-cadherin, or any combination thereof.

The term “precursor cell” as used herein has its plain and ordinary meaning as understood in light of the specification and encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment. Precursor cells include embryonic stem cells (ESC), embryonic carcinoma cells (ECs), and epiblast stem cells (EpiSC).

In some embodiments, one step can include obtaining stem cells that are pluripotent or can be induced to become pluripotent. In some embodiments, pluripotent stem cells are derived from embryonic stem cells, which are in turn derived from totipotent cells of the early mammalian embryo and are capable of unlimited, undifferentiated proliferation in vitro. Embryonic stem cells are pluripotent stem cells derived from the inner cell mass of the blastocyst, an early-stage embryo. Methods for deriving embryonic stem cells from blastocytes are well known in the art. It would be understood by one of skill in the art that the methods and systems described herein are applicable to any stem cells.

Additional stem cells that can be used in embodiments in accordance with the present disclosure include but are not limited to those provided by or described in the database hosted by the National Stem Cell Bank (NSCB), Human Embryonic Stem Cell Research Center at the University of California, San Francisco (UCSF); WISC cell Bank at the Wi Cell Research Institute; the University of Wisconsin Stem Cell and Regenerative Medicine Center (IJW-SCRMC); Novocell, Inc. (San Diego, Calif.); Cellartis AB (Goteborg, Sweden); ES Cell International Pte Ltd (Singapore); Techmon at the Israel Institute of Technology (Haifa, Israel); and the Stem Cell Database hosted by Princeton University and the University of Pennsylvania. Exemplary embryonic stem cells that can be used in embodiments in accordance with the present disclosure include but are not limited to SA01 (SA001); SA02 (SA002); ESDI (HES-1); ES02 (HES-2); ES03 (HES-3); ES04 (HES-4); ES05 (HES-5); ES06 (HES-6); BG01 (BGN-01); BG02 (BGN-02); BG03 (BGN-03); TE03 (13); TE04 (14); TE06 (16); UCO1 (HSF1); UC06 (HSF6); WA01 (HI); WA07 (H7); WA09 (H9); WA13 (HI 3); WA14 (HI 4). Exemplary human pluripotent cell lines include but are not limited to TkDA3-4, 1231 A3, 317-D6, 317-A4, CDH1, 5-T-3, 3-34-1, NAFLD27, NAFLD77, NAFLD150, WD90, WD91, WD92, 1.20012. C213, 1383D6, FF, or 317-12 cells.

In developmental biology, cellular differentiation is the process by which a less specialized cell becomes a more specialized cell type. As used herein, the term “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

In some embodiments, an adenovirus can be used to transport the requisite four genes, resulting in iPSCs substantially identical to embryonic stem cells. Since the adenovirus does not combine any of its own genes with the targeted host, the danger of creating tumors is eliminated. In some embodiments, non-viral based technologies are employed to generate iPSCs. In some embodiments, reprogramming can be accomplished via plasmid without any virus transfection system at all, although at very low efficiencies. In other embodiments, direct delivery of proteins is used to generate iPSCs, thus eliminating the need for viruses or genetic modification. In some embodiment, generation of mouse iPSCs is possible using a similar methodology: a repeated treatment of the cells with certain proteins channeled into the cells via poly-arginine anchors was sufficient to induce pluripotency. In some embodiments, the expression of pluripotency induction genes can also be increased by treating somatic cells with FGF2 under low oxygen conditions.

The term “feeder cell” as used herein has its plain and ordinary meaning as understood in light of the specification and refers to cells that support the growth of pluripotent stem cells, such as by secreting growth factors into the medium or displaying on the cell surface. Feeder cells are generally adherent cells and may be growth arrested. For example, feeder cells are growth-arrested by irradiation (e.g. gamma rays), mitomycin-C treatment, electric pulses, or mild chemical fixation (e.g. with formaldehyde or glutaraldehyde). However, feeder cells do not necessarily have to be growth arrested. Feeder cells may serve purposes such as secreting growth factors, displaying growth factors on the cell surface, detoxifying the culture medium, or synthesizing extracellular matrix proteins. In some embodiments, the feeder cells are allogeneic or xenogeneic to the supported target stem cell, which may have implications in downstream applications. In some embodiments, the feeder cells are mouse cells. In some embodiments, the feeder cells are human cells. In some embodiments, the feeder cells are mouse fibroblasts, mouse embryonic fibroblasts, mouse STO cells, mouse 3T3 cells, mouse SNL 76/7 cells, human fibroblasts, human foreskin fibroblasts, human dermal fibroblasts, human adipose mesenchymal cells, human bone marrow mesenchymal cells, human amniotic mesenchymal cells, human amniotic epithelial cells, human umbilical cord mesenchymal cells, human fetal muscle cells, human fetal fibroblasts, or human adult fallopian tube epithelial cells. In some embodiments, conditioned medium prepared from feeder cells is used in lieu of feeder cell co-culture or in combination with feeder cell co-culture. In some embodiments, feeder cells are not used during the proliferation of the target stem cells.

### Differentiation of PSCs

Known methods for producing definitive endoderm from pluripotent cells (e.g., iPSCs or ESCs) are applicable to the methods described herein. In some embodiments, pluripotent cells are derived from a morula. In some embodiments, pluripotent stem cells are stem cells. Stem cells used in these methods can include, but are not limited to, embryonic stem cells or induced pluripotent stem cells. Embryonic stem cells can be derived from the embryonic inner cell mass or from the embryonic gonadal ridges. Embryonic stem cells can originate from a variety of animal species including, but not limited to, various mammalian species including humans. In some embodiments, human embryonic stem cells are used to produce definitive endoderm. In some embodiments, iPSCs are used to produce definitive endoderm. In some embodiments, human iPSCs (hiPSCs) are used to produce definitive endoderm.

In some embodiments, PSCs, such as ESCs and iPSCs, undergo directed differentiation into embryonic germ layer cells, organ tissue progenitor cells, and then into tissue such as liver tissue or any other biological tissue. In some embodiments, the directed differentiation is done in a stepwise manner to obtain each of the differentiated cell types where molecules (e.g. growth factors, ligands, agonists, antagonists) are added sequentially as differentiation progresses. In some embodiments, the directed differentiation is done in a non-stepwise manner where molecules (e.g. growth factors, ligands, agonists, antagonists) are added at the same time. In some embodiments, directed differentiation is achieved by selectively activating certain signaling pathways in the PSCs or any downstream cells.

In some embodiments, one or more pathway is activated and/or one or more pathway is inhibited in the embryonic stem cells or germ cells or iPSCs for a time that is, is about, is at least, is at least about, is not more than, or is not more than about, 6 hours, 12 hours, 18 hours, 24 hours, 36 hours, 48 hours, 60 hours, 72 hours, 84 hours, 96 hours, 120 hours, 150 hours, 180 hours, 240 hours, 300 hours or any time within a range defined by any two of the aforementioned times, for example 6 hours to 300 hours, 24 hours to 120 hours, 48 hours to 96 hours, 6 hours to 72 hours, or 24 hours to 300 hours. In some embodiments, more than one small molecule compounds, activators, inhibitors, or growth factors are added in order to effect activation of one or more pathway and/or inhibition of one or more pathway. In these cases, the more than one small molecule compounds, activators, inhibitors, or growth factors can be added simultaneously or separately.

In some embodiments, directed differentiation is achieved by selectively activating one, two, three, four, five, six, seven, or more pathways and/or by inhibiting one, two, three, four, five, six, seven, or more pathways in the PSCs or any downstream cells, in one, two, three, four, five, six, seven, or more pathway activation and/or inhibition steps, wherein the one, two, three, four, five, six, seven, or more pathway activation and/or inhibition steps can occur over one, two, three, four, five, six, seven, or more time periods. In some embodiments, two, three, four, five, six, seven, or more time periods can be consecutive or non-consecutive.

In some embodiments, the one or more pathway comprises a signaling pathway. In some embodiments, activation and/or inhibition of a pathway in the embryonic stem cells or germ cells or iPSCs is accomplished via treatment with one or more small molecule compounds, activators, inhibitors, or growth factors for a time that is, is about, is at least, is at least about, is not more than, or is not more than about, 6 hours, 12 hours, 18 hours, 24 hours, 36 hours, 48 hours, 60 hours, 72 hours, 84 hours, 96 hours, 120 hours, 150 hours, 180 hours, 240 hours, 300 hours or any time within a range defined by any two of the aforementioned times, for example 6 hours to 300 hours, 24 hours to 120 hours, 48 hours to 96 hours, 6 hours to 72 hours, or 24 hours to 300 hours. In some embodiments, more than one small molecule compounds, activators, inhibitors, or growth factors are added. In these cases, the more than one small molecule compounds, activators, inhibitors, or growth factors can be added simultaneously or separately.

In some embodiments, activation and/or inhibition of a pathway in the embryonic stem cells or germ cells or iPSCs is accomplished via treatment with one or more small molecule compounds, activators, inhibitors, or growth factors at a concentration that is, is about, is at least, is at least about, is not more than, or is not more than about, 10 ng/ml, 20 ng/ml, 50 ng/ml, 75 ng/mL, 100 ng/mL, 120 ng/mL, 150 ng/mL, 200 ng/mL, 500 ng/ml, 1000 ng/ml, 1200 ng/ml, 1500 ng/mL, 2000 ng/mL, 5000 ng/ml, 7000 ng/ml, 10000 ng/ml, or 15000 ng/ml, or any concentration that is within a range defined by any two of the aforementioned concentrations, for example, 10 ng/ml to 15000 ng/ml, 100 ng/mL to 5000 ng/ml, 500 ng/ml to 2000 ng/mL, 10 ng/mL to 2000 ng/mL, or 1000 ng/ml to 15000 ng/mL. In some embodiments, concentration of the one or more small molecule compounds, activators, inhibitors, or growth factors is maintained at a constant level throughout the treatment. In some embodiments, concentration of the one or more small molecule compounds, activators, inhibitors, or growth factors is varied during the course of the treatment. In some embodiments, more than one small molecule compounds, activators, inhibitors, or growth factors are added. In these cases, the more than one small molecule compounds, activators, inhibitors, or growth factors can differ in concentrations.

In some embodiments, the ESCs or iPSCs, or the ESCs, germ cells, or iPSCs are cultured in growth media that supports the growth of stem cells. In some embodiments, the ESCs or iPSCs, or the ESCs, germ cells, or iPSCs are cultured in stem cell growth media. In some embodiments, the stem cell growth media is RPMI 1640, DMEM, DMEM/F12, or Advanced DMEM/F12. In some embodiments, the stem cell growth media comprises fetal bovine serum (FBS). In some embodiments, the stem cell growth media comprises FBS at a concentration that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20%, or any percentage within a range defined by any two of the aforementioned concentrations, for example 0% to 20%, 0.2% to 10%, 2% to 5%, 0% to 5%, or 2% to 20%. In some embodiments, the stem cell growth media does not contain xenogeneic components. In some embodiments, the growth media comprises one or more small molecule compounds, activators, inhibitors, or growth factors.

In some embodiments, populations of cells enriched in definitive endoderm cells are used. In some embodiments, the definitive endoderm cells are isolated or substantially purified. In some embodiments, the isolated or substantially purified definitive endoderm cells express one or more (e.g. at least 1, 3) of SOX17, FOXA2, or CXRC4 markers to a greater extent than one or more (e.g. at least 1, 3, 5) of OCT4, AFP, TM, SPARC, or SOX7 markers.

In some embodiments, pluripotent stem cells are prepared from somatic cells. In some embodiments, pluripotent stem cells are prepared from biological tissue obtained from a biopsy. In some embodiments, the pluripotent stem cells are cryopreserved. In some embodiments, the somatic cells are cryopreserved. In some embodiments, pluripotent stem cells are prepared from PBMCs. In some embodiments, human PSCs are prepared from human PBMCs. In some embodiments, pluripotent stem cells are prepared from cryopreserved PBMCs. In some embodiments, PBMCs are grown on a feeder cell substrate. In some embodiments, PBMCs are grown on a mouse embryonic fibroblast (MEF) feeder cell substrate. In some embodiments, PBMCs are grown on an irradiated MEF feeder cell substrate.

In some embodiments, stem cells are treated with one or more growth factors to differentiate to definitive endoderm cells. Such growth factors can include growth factors from the TGF-beta superfamily. In some embodiments, the one or more growth factors comprise the Nodal/Activin and/or the BMP subgroups of the TGF-beta superfamily of growth factors. In some embodiments, the one or more growth factors are selected from the group consisting of Nodal, Activin A, Activin B, BMP4, Wnt3a or combinations of any of these growth factors. In some embodiments, the stem cells are contacted with Activin A. In some embodiments, the stem cells are contacted with Activin A and BMP4.

In some embodiments, activin-induced definitive endoderm (DE) can further undergo anterior endoderm pattering, foregut specification and morphogenesis, dependent on FGF, Wnt, BMP, or retinoic acid, or any combination thereof, or on FGF, Wnt, BMP, or retinoic acid, or any combination thereof, and a liver culture system that promotes liver growth, morphogenesis and cytodifferentiation. In some embodiments, human PSCs are efficiently directed to differentiate in vitro into liver epithelium and mesenchyme. It will be understood that molecules such as growth factors can be added to any stage of the development to promote a particular type of hepatic tissue formation. In some embodiments, siRNA and/or shRNA targeting cellular constituents associated with the FGF, Wnt, BMP, or retinoic acid signaling pathways are used to inhibit or activate these pathways. It will further be understood by one of skill in the art that activating and/or inhibiting a pathway, such as, for example, Wnt signaling, FGF signaling, BMP signaling, etc., can be achieved by various means, so long as the downstream effect of pathway activation or inhibition is achieved. Thus, activating or inhibiting a signaling pathway can include providing a signaling pathway activator or signaling pathway inhibitor, as appropriate; alternatively, this can also include providing a protein or other compound or component which achieves or mimics the downstream effect of the signaling pathway activation or inhibition.

It will further be understood by one of skill in the art that altering the concentration, expression or function of, e.g., one or more Wnt signaling proteins in combination with altering the concentration, expression, or function of, e.g., one or more FGF proteins can give rise to directed differentiation in accordance with the present disclosure. In some embodiments, cellular constituents associated with the FGF, Wnt, or retinoic acid (RA) signaling pathways, or with the FGF, Wnt, BMP, or retinoic acid (RA) signaling pathways, for example, natural inhibitors, antagonists, activators, or agonists of the pathways can be used to result in inhibition or activation of the FGF, Wnt, or retinoic acid signaling pathways, or of the FGF, Wnt, BMP, or retinoic acid signaling pathways. In some embodiments, siRNA and/or shRNA targeting cellular constituents associated with the FGF, Wnt, or retinoic acid signaling pathways, or the FGF, Wnt, BMP, or retinoic acid signaling pathways, are used to inhibit or activate these pathways.

In some embodiments, pluripotent stem cells, definitive endoderm, posterior foregut spheroids, or downstream liver cell types are contacted with a Wnt signaling pathway activator or Wnt signaling pathway inhibitor. In some embodiments, the Wnt signaling pathway activator comprises a Wnt protein. In some embodiments, the Wnt protein comprises a recombinant Wnt protein. In some embodiments, the Wnt signaling pathway activator comprises Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, or any combination thereof. In some embodiments, the Wnt signaling pathway activator comprises a GSK3 signaling pathway inhibitor. In some embodiments, the Wnt signaling pathway activator comprises CHIR99021, CHIR 98014, AZD2858, BIO, AR-A014418, SB 216763, SB 415286, aloisine, indirubin, alsterpaullone, kenpaullone, lithium chloride, TDZD 8, or TWS119, or any combination thereof. In some embodiments, the Wnt signaling pathway inhibitor comprises C59, PNU 74654, KY-02111, PRI-724, FH-535, DIF-1, or XAV939, or any combination thereof. In some embodiments, the cells are not treated with a Wnt signaling pathway activator or Wnt signaling pathway inhibitor. The Wnt signaling pathway activator or Wnt signaling pathway inhibitor provided herein may be used in combination with any of the other growth factors, signaling pathway activators, or signaling pathway inhibitors provided herein.

In some embodiments, pluripotent stem cells, definitive endoderm, posterior foregut spheroids, or downstream liver cell types are contacted with an FGF signaling pathway activator. In some embodiments, the FGF signaling pathway activator comprises an FGF protein. In some embodiments, the FGF protein comprises a recombinant FGF protein. In some embodiments, the FGF signaling pathway activator comprises one or more of FGF1, FGF2, FGF3, FGF4, FGF4, FGF5, FGF6, FGF7, FGF8, FGF8, FGF9, FGF10, FGF11, FGF12, FGF13, FGF14, FGF15 (FGF19, FGF15/FGF19), FGF16, FGF17, FGF18, FGF20, FGF21, FGF22, or FGF23. In some embodiments, the cells are not treated with an FGF signaling pathway activator. The FGF signaling pathway activator provided herein may be used in combination with any of the other growth factors, signaling pathway activators, or signaling pathway inhibitors provided herein.

In some embodiments, pluripotent stem cells, definitive endoderm, posterior foregut spheroids, or downstream liver cell types are contacted with a BMP signaling pathway activator or BMP signaling pathway inhibitor. In some embodiments, the BMP signaling pathway activator comprises a BMP protein. In some embodiments, the BMP protein is a recombinant BMP protein. In some embodiments, the BMP signaling pathway activator comprises BMP1, BMP2, BMP3, BMP4, BMP5, BMP6, BMP7, BMP8a, BMP8b, BMP10, BMP11, BMP15, IDE1, or IDE2, or any combination thereof. In some embodiments, the BMP signaling pathway inhibitor comprises Noggin, RepSox, LY364947, LDN-193189, SB431542, or any combination thereof. In some embodiments, the cells are not treated with a BMP signaling pathway activator or BMP signaling pathway inhibitor. The BMP signaling pathway activator or BMP signaling pathway inhibitor provided herein may be used in combination with any of the other growth factors, signaling pathway activators, or signaling pathway inhibitors provided herein.

In some embodiments, pluripotent stem cells, definitive endoderm, posterior foregut spheroids, or downstream liver cell types are contacted with a retinoic acid signaling pathway activator or retinoic acid signaling pathway inhibitor. In some embodiments, the retinoic acid signaling pathway activator comprises retinoic acid, all-trans retinoic acid, 9-cis retinoic acid, CD437, EC23, BS 493, TTNPB, or AM580, or any combination thereof. In some embodiments, the retinoic acid signaling pathway inhibitor comprises guggulsterone. In some embodiments, the cells are not treated with a retinoic acid signaling pathway activator or retinoic acid signaling pathway inhibitor. The retinoic acid signaling pathway activator or retinoic acid signaling pathway inhibitor provided herein may be used in combination with any of the other growth factors, signaling pathway activators, or signaling pathway inhibitors provided herein.

In some embodiments, pluripotent stem cells are converted into liver cell types via a “one step” process. For example, one or more molecules that can differentiate pluripotent stem cells into DE culture (e.g., Activin A) are combined with additional molecules that can promote directed differentiation of DE culture (e.g., FGF4, CHIR99021, LDN-193189, RA) to directly treat pluripotent stem cells.

In some embodiments, pluripotent stem cells (e.g., ESCs or iPSCs) are expanded in cell culture. In some embodiments, pluripotent stem cells are expanded in a basement membrane matrix. In some embodiments, pluripotent stem cells are expanded in Matrigel. In some embodiments, the pluripotent stem cells are expanded in cell culture comprising a ROCK inhibitor (e.g. Y-27632). In some embodiments, the iPSCs are differentiated into definitive endoderm cells. In some embodiments, the pluripotent stem cells are differentiated into definitive endoderm cells by contacting the pluripotent stem cells with Activin A, BMP4, or both. In some embodiments, the pluripotent stem cells are contacted with a concentration of Activin A that is, is about, is at least, is at least about, is not more than, or is not more than about, 10, 20, 30, 40, 50, 60, 70, 80, 90, 100, 110, 120, 130, 140, 150, 160, 170, 180, 190, or 200 ng/mL, or any concentration of Activin A within a range defined by any two of the aforementioned concentrations, for example, 10 to 200 ng/mL, 10 to 100 ng/mL, 100 to 200 ng/mL, or 50 to 150 ng/mL. In some embodiments, the pluripotent stem cells are contacted with Activin A at a concentration of 100 ng/ml or about 100 ng/mL. In some embodiments, the pluripotent stem cells are contacted with a concentration of BMP4 that is, is about, is at least, is at least about, is not more than, or is not more than about, 1, 2, 3, 4, 5, 6, 7, 8, 9, 10, 20, 30, 40, 50, 60, 70, 80, 90, 100, 110, 120, 130, 140, 150, 160, 170, 180, 190, or 200 ng/mL, or any concentration of BMP4 within a range defined by any two of the aforementioned concentrations, for example, 1 to 200 ng/mL, 1 to 100 ng/ml, 25 to 200 ng/ml, 1 to 80 ng/mL, or 25 to 100 ng/mL. In some embodiments, the pluripotent stem cells are contacted with BMP4 at a concentration of 50 ng/ml or about 50 ng/mL.

In some embodiments, the PSCs are differentiated into definitive endoderm cells. In some embodiments, the PSCs are differentiated into posterior foregut cells. In some embodiments, the PSCs are differentiated into one or more liver spheroids. In some embodiments, the PSCs are differentiated into a liver organoid.

In some embodiments, any of the cells disclosed herein may be cryopreserved for later use. The cells can be cryopreserved according to methods generally known in the art, optionally including one or more cryoprotectants.

### Gene Editing

Embodiments of the disclosure can include PSCs, iPSCs, definitive endoderm cells, posterior foregut spheroids, or organoids which have been or which can be genetically modified or edited according to methods known in the art. For example, gene editing using CRISPR nucleases such as Cas9 are explored in PCT Publications WO 2013/176772, WO 2014/093595, WO 2014/093622, WO 2014/093655, WO 2014/093712, WO 2014/093661, WO 2014/204728, WO 2014/204729, WO 2015/071474, WO 2016/115326, WO 2016/141224, WO 2017/023803, and WO 2017/070633, each of which is hereby expressly incorporated by reference in its entirety.

### Liver-Related Diseases and Disorders

The liver organoids of the disclosure can be used in treatment and/or studying or modeling liver-related diseases and disorders, for which their neural character and functionality is particularly advantageous and renders them applicable to a wide range of conditions. In some embodiments, the methods include administering any of the liver organoids or liver cells disclosed herein. Also disclosed herein are the liver organoids or liver cells disclosed herein for use in the manufacture of a medicament for the treatment of a liver-related disease or disorder. Also disclosed herein are the liver organoids or liver cells disclosed herein for use in the treatment of a liver-related disease or disorder in a subject in need thereof.

Liver-related diseases and disorders relevant to the disclosure can include conditions such as liver dysfunction and/or failure (e.g. hyperammonemia and/or hyperbilirubinemia, and the like), hepatitis (e.g. hepatitis A, hepatitis B, hepatitis C, hepatitis D, hepatitis E, hepatitis G, hepatitis TT, and/or autoimmune hepatitis, and the like), viral hepatitis, cholangitis, fibrosis, hepatic encephalopathy, hepatic porphyria, cirrhosis, cancer, drug-induced cholestasis, metabolic disease (e.g. metabolic dysfunction-associated liver disease (MASLD), MetALD, nonalcoholic fatty liver disease (NAFLD), metabolic dysfunction-associated steatohepatitis (MASH), and the like), autoimmune liver disease, Wilson's disease, metabolic-associated fatty liver disease, hyperammonemia, hyperbilirubinemia, Crigler-Najjar Syndrome, urea cycle disorders, Wolman disease, hepatic cancer, hepatoblastoma, drug-induced liver injury (DILI), glycogen storage disease, hemorrhagic disease, hepatic cyst, glucogenesis, and/or alcohol-associated liver disease. One skilled in the art will appreciate other liver-related diseases and conditions for which the liver organoids disclosed herein could have relevance.

For example, the liver organoid can be transplanted into a subject having liver dysfunction and/or failure, where the transplanted liver organoids engraft onto the liver of the subject. Following transplantation, the subject can have reduced serum bilirubin and/or ammonia levels, and/or increased serum protein albumin, and/or improved symptoms of biliary stricture and/or liver regeneration, and can also have increased survival rate.

For example, these liver organoids can be used an in vitro human model system for studying hepatocyte function and developmental divergence, studying liver-related disease, identifying and/or screening for therapeutic targets, and/or identifying therapeutic compounds and/or compositions effective in treating a liver-related disease or disorder. Accordingly, the liver organoids of the disclosure can allow for new developments in liver disease treatment and study.

### Pharmaceutical Compositions

Embodiments of the disclosure can include pharmaceutical compositions. Such pharmaceutical compositions can include one or more additional pharmaceutically acceptable components, which can include carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or earner approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or “carrier” can refer to a diluent, adjuvant, excipient, or vehicle with which the pharmaceutical composition is administered. Such pharmaceutical diluent, excipient, and/or earners can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, ammo acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), and PLURONICS®. The composition, if desired, can also contain minor amounts of wetting, bulking, emulsifying agents, or pH buffering agents. These compositions can take the form of solutions, suspensions, emulsion, sustained release formulations and the like. The formulation should suit the mode of administration.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, b-propiolactone, gelatin, cell debris, nucleic acids, peptides, ammo acids, or growth medium components or any combination thereof. The amount of the excipient may be found in composition at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

Pharmaceutical compositions can include one or more “pharmaceutically acceptable salts”, which can include relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; ammo acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl ammoethane.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular, subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration.

As used herein, a “carrier” has its plain and ordinary meaning as understood in light of the specification and can refer to a compound, particle, solid, semi-solid, liquid, or diluent that facilitates the passage, delivery and/or incorporation of a compound to cells, tissues and/or bodily organs.

As used herein, a “diluent” has its plain and ordinary meaning as understood in light of the specification and can refer to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

### Dosage and Administration Routes

Embodiments of the disclosure can include methods of administering or treating an animal, which can involve administering an amount of at least one treatment, that is effective to treat the disease, condition, or disorder that the organism has, or is suspected of having, or is susceptible to, or to bring about a desired physiological effect. In some embodiments, the disease, condition, or disorder can be a liver-related disease or disorder.

In some embodiments, at least one treatment can include a composition or pharmaceutical composition, which can be administered to an animal (e.g., mammals, primates, monkeys, or humans) in an amount of about 0.005 to about 50 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 0.5 mg/kg, about 1 mg/kg, about 3 mg/kg, about 5 mg/kg, about 5.5 mg/kg, about 6 mg/kg, about 6.5 mg/kg, about 7 mg/kg, about 7.5 mg/kg, about 8 mg/kg, about 10 mg/kg, about 12 mg/kg, or about 15 mg/kg. In regard to some conditions, the dosage can be about 0.5 mg/kg human body weight or about 6.5 mg/kg human body weight. In some instances, some subjects (e.g., mammals, mice, rabbits, feline, porcine, or canine) can be administered a dosage of about 0.005 to about 50 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 1 mg/kg, about 5 mg/kg, about 10 mg/kg, about 20 mg/kg, about 30 mg/kg, about 40 mg/kg, about 50 mg/kg, about 80 mg/kg, about 100 mg/kg, or about 150 mg/kg. Of course, those skilled in the art will appreciate that it is possible to employ many concentrations in the methods of the present disclosure, and using, in part, the guidance provided herein, will be able to adjust and test any number of concentrations in order to find one that achieves the desired result in a given circumstance. In some embodiments, a dose or a therapeutically effective dose of a compound disclosed herein will be that which is sufficient to achieve a plasma concentration of the compound or its active metabolite(s) within a range set forth herein, e.g., about 1-10 nM, 10-100 nM, 0.1-1 μM, 1-10 μM, 10-100 μM, 100-200 μM, 200-500 μM, or even 500-1000 μM, preferably about 1-10 nM, 10-100 nM, or 0.1-1 μM.

In other embodiments, a treatment can be administered in combination with one or more other therapeutic agents for a given disease, condition, or disorder.

The compounds and pharmaceutical compositions are preferably prepared and administered in dose units. Solid dose units are tablets, capsules and suppositories. For treatment of a subject, depending on activity of the compound, manner of administration, nature and severity of the disease or disorder, age and body weight of the subject, different daily doses can be used.

Under certain circumstances, however, higher or lower daily doses can be appropriate. The administration of the daily dose can be carried out both by single administration in the form of an individual dose unit or else several smaller dose units and also by multiple administrations of subdivided doses at specific intervals.

A treatment can be administered locally or systemically in a therapeutically effective dose. Amounts effective for this use will, of course, depend on the severity of the disease or disorder and the weight and general state of the subject. Typically, dosages used in vitro can provide useful guidance in the amounts useful for in situ administration of the pharmaceutical composition, and animal models can be used to determine effective dosages for treatment of particular disorders.

Various considerations are described, e.g., in Langer, 1990, Science, 249:1527; Goodman and Gilman's (eds.), 1990, Id., each of which is herein incorporated by reference and for all purposes. Dosages for parenteral administration of active pharmaceutical agents can be converted into corresponding dosages for oral administration by multiplying parenteral dosages by appropriate conversion factors. As to general applications, the parenteral dosage in mg/mL times 1.8=the corresponding oral dosage in milligrams (“mg”). As to oncology applications, the parenteral dosage in mg/mL times 1.6=the corresponding oral dosage in mg. An average adult weighs about 70 kg. See e.g., Miller-Keane, 1992, Encyclopedia & Dictionary of Medicine, Nursing & Allied Health, 5th Ed., (W. B. Saunders Co.), pp. 1708 and 1651.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

In some embodiments, the administration can include a unit dose of one or more treatments in combination with a pharmaceutically acceptable carrier and, in addition, can include other medicinal agents, pharmaceutical agents, carriers, adjuvants, diluents, and excipients. In certain embodiments, the carrier, vehicle or excipient can facilitate administration, delivery and/or improve preservation of the composition. In other embodiments, the one or more carriers, include but are not limited to, saline solutions such as normal saline, Ringer's solution, PBS (phosphate-buffered saline), and generally mixtures of various salts including potassium and phosphate salts with or without sugar additives such as glucose. Carriers can include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents. In other embodiments, the one or more excipients can include, but are not limited to water, saline, dextrose, glycerol, ethanol, or the like, and combinations thereof. Nontoxic auxiliary substances, such as wetting agents, buffers, or emulsifiers may also be added to the composition. Oral formulations can include such normally employed excipients as, for example, pharmaceutical grades of mannitol, lactose, starch, magnesium stearate, sodium saccharine, cellulose, and magnesium carbonate.

The quantity of active component in a unit dose preparation can be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

A treatment can be administered to subjects by any number of suitable administration routes or formulations. The treatment, such as an immunotherapy, can also be used to treat subjects for a variety of diseases. Subjects include but are not limited to mammals, primates, monkeys (e.g., macaque, rhesus macaque, or pig tail macaque), humans, canine, feline, bovine, porcine, avian (e.g., chicken), mice, rabbits, and rats. In particular embodiments described herein, the subject is a human.

The route of administration of the compounds of the treatments described herein can be of any suitable route. Administration routes can be, but are not limited to the oral route, the parenteral route, the cutaneous route, the nasal route, the rectal route, the vaginal route, and the ocular route. In other embodiments, administration routes can be parenteral administration, a mucosal administration, intravenous administration, subcutaneous administration, topical administration, intradermal administration, oral administration, sublingual administration, intranasal administration, or intramuscular administration. The choice of administration route can depend on the compound identity (e.g., the physical and chemical properties of the compound) as well as the age and weight of the animal, the particular disease (e.g., type of cancer), and the severity of the disease (e.g., stage or severity of cancer). Of course, combinations of administration routes can be administered, as desired.

Some embodiments of the disclosure include a method for providing a subject with a treatment which comprises one or more administrations of one or more compositions; the compositions may be the same or different if there is more than one administration.

### Toxicity

The ratio between toxicity and therapeutic effect for a particular treatment is its therapeutic index and can be expressed as the ratio between LD50 (the amount of compound lethal in 50% of the population) and ED50 (the amount of compound effective in 50% of the population). Compounds that exhibit high therapeutic indices are preferred. Therapeutic index data obtained from in vitro assays, cell culture assays and/or animal studies can be used in formulating a range of dosages for use in humans. The dosage of such compounds preferably lies within a range of plasma concentrations that include the ED50 with little or no toxicity. The dosage can vary within this range depending upon the dosage form employed and the route of administration utilized. See, e.g. Fingl et al., In: THE PHARMACOLOGICAL BASIS OF THERAPEUTICS, Ch.1, p.1, 1975. The exact formulation, route of administration, and dosage can be chosen by the individual practitioner in view of the patient's condition and the particular method in which the compound is used. For in vitro formulations, the exact formulation and dosage can be chosen by the individual practitioner in view of the patient's condition and the particular method in which the compound is used.

### Compositions

In some embodiments, also provided herein are compositions for performing any of the methods disclosed herein. In some embodiments, also provided herein are compositions produced according to processes provided in any of the methods disclosed herein. It is expressly contemplated that, in certain embodiments, any method or composition described herein can be implemented with respect to any other method or composition described herein and that different embodiments may be combined.

In some embodiments, provided herein are cell compositions in the form of a three-dimensional artificial liver organoid, where the liver organoid includes sympathetic neurons, or where the liver organoid includes a reduced number, or is absent, of sympathetic neurons, as described herein.

In some embodiments, provided herein are cell compositions in the form of a three-dimensional artificial liver organoid comprising hepatic stellate cells, hepatocytes, and sympathetic neurons. Such cell compositions including three-dimensional artificial liver organoids can include comprising hepatic stellate cells, hepatocytes, and sympathetic neurons, such as the liver organoid containing sympathetic neurons as described herein.

In some embodiments, provided herein are ex vivo compositions in the form of a three-dimensional artificial liver organoid comprising hepatic stellate cells, hepatocytes, and sympathetic neurons. Such cell compositions including ex vivo three-dimensional artificial liver organoids can include comprising hepatic stellate cells, hepatocytes, and sympathetic neurons, such as the liver organoid containing sympathetic neurons as described herein.

In some embodiments, provided herein are compositions, such as cell compositions and/or liver organoids including sympathetic neurons, or sympathetic nerves, as well as hepatic stellate cells, hepatocytes. In some embodiments, provided herein are compositions, such as cell compositions and/or liver organoids including sympathetic neurons, or sympathetic nerves, that further comprise hepatoblasts. In some embodiments, provided herein are compositions, such as cell compositions and/or liver organoids including sympathetic neurons, or sympathetic nerves, that further comprise cholangiocytes, endothelial cells, macrophages, stellate cells, mesenchymal cells, epithelial cells, Schwann cells, and/or neural crest cells. In some embodiments, the compositions, such as cell compositions and/or liver organoids including sympathetic neurons, or sympathetic nerves, include Schwann cells, neural crest cells, and/or other nerve cells. In some embodiments, the hepatocytes self-assemble into the three-dimensional artificial liver organoids including sympathetic neurons, or sympathetic nerves. In some embodiments, the three-dimensional artificial liver organoid including sympathetic neurons, or sympathetic nerves, includes a structure with a single lumen. In some embodiments, the three-dimensional artificial liver organoid including sympathetic neurons, or sympathetic nerves, does not contain hematopoietic tissue and/or acquired immune cells.

In some embodiments, provided herein are compositions, such as cell compositions and/or liver organoids including sympathetic neurons, or sympathetic nerves, as well as hepatic stellate cells, hepatocytes. In some embodiments, provided herein are compositions, such as cell compositions and/or liver organoids with a reduced number, or absent, of sympathetic neurons, or sympathetic nerves, that further comprise hepatoblasts. In some embodiments, provided herein are compositions, such as cell compositions and/or liver organoids with a reduced number, or absent, of sympathetic neurons, or sympathetic nerves, that further comprise cholangiocytes, endothelial cells, macrophages, stellate cells, mesenchymal cells, epithelial cells, Schwann cells, and/or neural crest cells. In some embodiments, the compositions, such as cell compositions and/or liver organoids including sympathetic neurons, or sympathetic nerves, include Schwann cells, neural crest cells, and/or other nerve cells. In some embodiments, the hepatocytes self-assemble into the three-dimensional artificial liver organoids with a reduced number, or absent, of sympathetic neurons, or sympathetic nerves. In some embodiments, the three-dimensional artificial liver organoid with a reduced number, or absent, of sympathetic neurons, or sympathetic nerves, includes a structure with a single lumen. In some embodiments, the three-dimensional artificial liver organoid with a reduced number, or absent, of sympathetic neurons, or sympathetic nerves, does not contain hematopoietic tissue and/or acquired immune cells.

In some embodiments compositions provided herein may comprise cell populations differentiated from pluripotent stem cells. In some embodiments compositions provided herein may comprise cell populations differentiated from induced pluripotent stem cells (iPSCs). In some embodiments, compositions provided herein comprise exogenously added and/or transgenically produced ascorbate (vitamin C), and/or exogenously provided bilirubin. In some embodiments, provided herein are compositions comprising hepatocytes that are engineered to express a heterologous functional GULO protein, and ascorbate is produced by the hepatocytes. In some embodiments, provided herein are compositions comprising exogenously provided bilirubin at a concentration of about 0.1 mg/L, 0.2 mg/L, 0.3 mg/L, 0.4 mg/L, 0.5 mg/L, 0.6 mg/L, 0.7 mg/L, 0.8 mg/L, 0.9 mg/L, 1 mg/L, 1.1 mg/L, 1.2 mg/L, 1.3 mg/L, 1.4 mg/L, 1.5 mg/L, 1.6 mg/L, 1.7 mg/L, 1.8 mg/L, 1.9 mg/L, 2 mg/L, 2.1 mg/L, 2.2 mg/L, 2.3 mg/L, 2.4 mg/L, 2.5 mg/L, 2.6 mg/L, 2.7 mg/L, 2.8 mg/L, 2.9 mg/L, or 3 mg/L. In some embodiments, provided herein are compositions comprising exogenously provided bilirubin at a concentration of about 1 mg/L.

In some embodiments, provided herein are compositions including liver organoids comprising multiple cell types, including at least hepatic stellate cells, hepatocytes, and neural cells. In some embodiments, the compositions including liver organoids include about 1%-75%, 2%-65%, 5%-60%, 5-25%, or 10-20%, neural cells; about 10%-90%, 15%-75%, or 15%-65%, epithelial cells; and/or about 10%-90%, 15%-75%, or 15%-60%, hepatic stellate cells and hematopoietic cells. In some embodiments, provided herein are compositions including liver organoids comprising at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, or greater, neural cells; wherein the liver organoid comprises at least about 10%, 20%, 25%, 30%, 35%, 40%, 45%, 50%, 55%, 60%, 65%, 70%, 75%, 80%, 85%, 90%, or greater, epithelial cells; and/or wherein the liver organoid comprises at least about 10%, 20%, 25%, 30%, 35%, 40%, 45%, 50%, 55%, 60%, 65%, 70%, 75%, 80%, 85%, 90%, or greater, hepatic stellate cells and hematopoietic cells. In some embodiments, provided herein are compositions including liver organoids comprising at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, or more, neural cells.

In some embodiments, compositions provided herein are in vitro compositions, created outside of a multicellular living organism. In some embodiments, compositions provided herein may be introduced into a multicellular living organism. In some embodiments, compositions provided herein comprise exogenously provided components, reagents, and/or conditions. In some embodiments, compositions provided herein comprise exogenously provided components, reagents, and/or conditions that mimic in vivo characteristics desirable for inducing specific cellular differentiation and/or organoid organization.

In some embodiments, provided herein are compositions comprising a tissue culture surface that is coated with a basement membrane matrix or component thereof. In some embodiments, a basement membrane matrix or component thereof does not comprise non-human animal components. In some embodiments, a basement membrane matrix or component thereof does not comprise non-human animal components such that the basement membrane matrix or component thereof is xenogeneic to humans. In some embodiments, a basement membrane matrix or component thereof is not isolated from murine Engelbreth-Holm-Swarm (EHS) sarcoma cells, is not Matrigel®, is not Cultrex®, and/or is not Geltrex®. In some embodiments, a basement membrane matrix or component thereof comprises human laminin, collagen IV, entactin, perlecan, fibrin, and/or hydrogel.

In some embodiments, provided herein are compositions that include an exogenous TGF-b pathway inhibitor. In some embodiments, an exogenous TGF-b pathway inhibitor comprises, consists essentially of, or consists of A83-01, RepSox, LY365947, and/or SB431542. In some embodiments, an exogenous TGF-b pathway inhibitor comprises, consists essentially of, or consists of TGF-b pathway inhibitor A83-01. In some embodiments, a composition comprises a TGF-b pathway inhibitor at a concentration of, or of about, 100, 200, 300, 400, 500, 600, 700, 800, 900, or 1000 nM, or any concentration within a range defined by any two of the aforementioned concentrations. In some embodiments, a composition comprises a TGF-b pathway inhibitor at a concentration of, or of about, 500 nM.

In some embodiments, provided herein are compositions that include an exogenous FGF pathway activator. In some embodiments, a composition comprises an exogenous FGF pathway activator that comprises, consists essentially of, or consists of FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF19, FGF20, FGF21, FGF22, and/or FGF23. In some embodiments, an exogenous FGF pathway activator comprises, consists essentially of, or consists of FGF2. In some embodiments, a composition comprises a FGF pathway activator at a concentration of, or of about 1, 2, 3, 4, 5, 6, 7, 8, 9, or 10 ng/mL, or any concentration within a range defined by any two of the aforementioned concentrations. In some embodiments, a composition comprises a FGF pathway activator at a concentration of, or of about 5 ng/mL.

In some embodiments, provided herein are compositions that include an exogenous Wnt pathway activator. In some embodiments, a composition comprises an exogenous Wnt pathway activator that comprises, consists essentially of, or consists of Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, CHIR99021, CHIR 98014, AZD2858, BIO, AR-A014418, SB 216763, SB 415286, aloisine, indirubin, alsterpaullone, kenpaullone, lithium chloride, TDZD 8, and/or TWS119. In some embodiments, a composition comprises an exogenous Wnt pathway activator that comprises, consists essentially of, or consists of CHIR99021. In some embodiments, a composition comprises a Wnt pathway activator at a concentration of, or of about, 1, 1.5, 2, 2.5, 3, 3.5, 4, 4.5, 5, 5.5, 6, 6.5, 7, 7.5, or 8 μM, or any concentration within a range defined by any two of the aforementioned concentrations. In some embodiments, In some embodiments, a composition comprises a Wnt pathway activator at a concentration of, or of about, 3 μM.

In some embodiments, provided herein are compositions that include an exogenous VEGF pathway activator. In some embodiments, a composition comprises an exogenous VEGF pathway activator that comprises, consists essentially of, or consists of VEGF and/or GS4012. In some embodiments, a composition comprises an exogenous VEGF pathway activator that comprises, consists essentially of, or consists of VEGF. In some embodiments, a composition comprises a VEGF pathway activator at a concentration of, or of about 1, 2, 3, 4, 5, 6, 7, 8, 9, 10, 11, 12, 13, 14, 15, 16, 17, 18, 19, or 20 ng/ml, or any concentration within a range defined by any two of the aforementioned concentrations. In some embodiments, a composition comprises a VEGF pathway activator at a concentration of, or of about 10 ng/mL.

In some embodiments, provided herein are compositions that include an exogenous EGF. In some embodiments, provided herein are compositions that do not include an exogenous EGF. In some embodiments, provided herein are compositions comprising EGF at a concentration of, or of about, 10, 11, 12, 13, 14, 15, 16, 17, 18, 19, 20, 21, 22, 23, 24, 25, 26, 27, 28, 29, or 30 ng/ml, or any concentration within a range defined by any two of the aforementioned concentrations. In some embodiments, provided herein are compositions comprising EGF at a concentration of, or of about, 20 ng/mL.

In some embodiments, provided herein are compositions that include exogenous and/or transgenically produced ascorbic acid. In some embodiments, provided herein are compositions that do not include exogenous and/or transgenically produced ascorbic acid. In some embodiments, provided herein are compositions comprising ascorbic acid at a concentration of, or of about, 10, 20, 30, 40, 50, 60, 70, 80, 90, or 100 μg/mL or any concentration within a range defined by any two of the aforementioned concentrations. In some embodiments, provided herein are compositions comprising ascorbic acid at a concentration of, or of about, 50 μg/mL.

In some embodiments, provided herein are compositions that include a ROCK inhibitor. In some embodiments, provided herein are compositions that do not include a ROCK inhibitor. In some embodiments, a ROCK inhibitor comprises, consists essentially of, or consists of Y-27632. In some embodiments, provided herein are compositions comprising a ROCK inhibitor at a concentration of, or of about, 1, 2, 3, 4, 5, 6, 7, 8, 9, 10, 11, 12, 13, 14, 15, 16, 17, 18, 19, or 20 μM, or any concentration within a range defined by any two of the aforementioned concentrations. In some embodiments, provided herein are compositions comprising a ROCK inhibitor at a concentration of, or of about, 10 μM.

In some embodiments, provided herein are compositions comprising liver organoids that have and/or that are being differentiated from stem cells. In some embodiments, provided herein are compositions comprising liver organoids that have and/or that are being differentiated from induced pluripotent stem cells. In some embodiments, provided herein are compositions comprising liver organoids comprising cells that have been passaged 1 time, 2 times, or 3 times. In some embodiments, provided herein are compositions comprising liver organoids comprising cells that have been passaged less than 4 times.

In some embodiments, provided herein are compositions comprising an FGF signaling pathway activator, a Wnt signaling pathway activator, an RA signaling pathway activator, and a BMP signaling pathway inhibitor. In some embodiments, the compositions further include iPSCs, PSCs, and/or posterior foregut cells and/or posterior foregut endoderm cells. In some embodiments, the compositions further include a culture medium.

In some embodiments, provided herein are compositions comprising FGF4, CHIR99021, and RA, and further comprising LDN-193189 and/or SB431542, optionally further comprising iPSCs, PSCs, and/or posterior foregut cells and/or posterior foregut endoderm cells. In some embodiments, the compositions further include a culture medium.

In some embodiments, provided herein are compositions comprising: a) posterior foregut cells and/or posterior foregut endoderm cells, liver organoids and/or mature liver organoids, and b) a medium, wherein the medium optionally comprises hepatocyte culture medium and is optionally supplemented with a cMET tyrosine kinase receptor agonist, an IL-6 family cytokine, and a corticosteroid, and wherein the composition optionally additionally comprises c) a retinoic acid pathway activator. In some embodiments, compositions provided herein comprise a cMET tyrosine kinase receptor agonist. In some embodiments, compositions provided herein comprise a cMET tyrosine kinase receptor agonist that comprises, consists essentially of, or consists of hepatocyte growth factor (HGF), PG-001, fosgonimeton, terevalefim, recombinant InlB321 protein, and/or an agonist c-Met antibody (e.g., LMH85).

In some embodiments, provided herein are compositions comprising an IL-6 family cytokine. In some embodiments, an IL-6 family cytokine comprises, consists essentially of, or consists of IL-6, Oncostatin M (OSM), leukemia inhibitory factor (LIF), cardiotrophin-1, ciliary neurotrophic factor (CTNF), and/or cardiotrophin-like cytokine (CLC).

In some embodiments, provided herein are compositions comprising a corticosteroid. In some embodiments, a corticosteroid comprises, consists essentially of, or consists of dexamethasone, beclometasone, betamethasone, fluocortolone, halometasone, and/or mometasone.

In some embodiments, provided herein are compositions comprising a hepatocyte culture media supplemented with HGF, OSM, and/or dexamethasone. In some embodiments, provided herein are compositions comprising a hepatocyte culture media supplemented with dexamethasone. In some embodiments, provided herein are compositions comprising a hepatocyte culture media supplemented with HGF. In some embodiments, provided herein are compositions comprising a hepatocyte culture media supplemented with OSM.

In some embodiments, provided herein are compositions comprising a retinoic acid pathway activator. In some embodiments, a retinoic acid pathway activator comprises, consists essentially of, or consists of retinoic acid, all-trans retinoic acid, 9-cis retinoic acid, CD437, EC23, BS 493, TTNPB, and/or AM580. In some embodiments, a retinoic acid pathway activator comprises, consists essentially of, or consists of retinoic acid. In some embodiments, compositions comprise a retinoic acid pathway activator at a concentration of, or of about, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 μM, or any concentration within a range defined by any two of the aforementioned concentrations. In some embodiments, compositions comprise a retinoic acid pathway activator at a concentration of, or of about, 2.0 μM.

In some embodiments, compositions comprise HGF. In some embodiments, compositions comprise HGF at a concentration of, or of about, 1, 2, 3, 4, 5, 6, 7, 8, 9, 10, 11, 12, 13, 14, 15, 16, 17, 18, 19, or 20 ng/mL, or any concentration within a range defined by any two of the aforementioned concentrations. In some embodiments, compositions comprise HGF at a concentration of, or of about 10 ng/ml.

In some embodiments, compositions comprise OSM. In some embodiments, compositions comprise OSM at a concentration of, or of about, 10, 11, 12, 13, 14, 15, 16, 17, 18, 19, 20, 21, 22, 23, 24, 25, 26, 27, 28, 29, or 30 ng/mL, or any concentration within a range defined by any two of the aforementioned concentrations. In some embodiments, compositions comprise OSM at a concentration of, or of about 20 ng/ml.

In some embodiments, compositions comprise dexamethasone. In some embodiments, compositions comprise dexamethasone at concentration of, or of about, 50, 60, 70, 80, 90, 100, 110, 120, 130, 140, 150, 160, 170, 180, 190, or 200 nM, or any concentration within a range defined by any two of the aforementioned concentrations. In some embodiments, compositions comprise dexamethasone at a concentration of, or of about 100 nM.

In some embodiments, compositions comprise exogenous bilirubin. In some embodiments, compositions comprise both exogenous bilirubin and endogenous bilirubin. In some embodiments, compositions comprise a low concentration of exogenous bilirubin. In some embodiments, a low concentration of exogenous bilirubin is at or near a human fetal physiological concentration of bilirubin. Human fetal bilirubin levels are thought to be generally around 1 mg/L (0.1 mg/dL), which rises rapidly to 3-10 mg/L (0.3-1.0 mg/dL) 24 hours after birth. In some embodiments, compositions comprise bilirubin, exogenous and/or endogenous, that is, is about, is less than, or is less than about: 0.1, 0.2, 0.3, 0.4, 0.5, 0.6, 0.7, 0.8, 0.9, 1, 1.25, 1.5, 1.75, 2.0, 2.25, 2.5, 2.75 or 3.0 mg/L, or at any concentration within a range defined by any two of the aforementioned concentrations, for example, 0.1 to 3 mg/L, 0.5 to 2.0 mg/L, 0.5 to 1.5 mg/L, 0.3 to 2.5 mg/L, or 0.5 to 1.75 mg/L; or 0.1, 0.2, 0.3, 0.4, 0.5, 0.6, 0.7, 0.8, 0.9, or 1 mg/L, or any concentration within a range defined by any two of the aforementioned concentrations, for example, 0.1 to 1 mg/L, 0.1 to 0.5 mg/L, 0.5 to 1 mg/L, 0.3 to 0.7 mg/L, or 0.4 to 0.6 mg/L. In some embodiments, compositions comprise exogenous bilirubin at a concentration that is, is about, is less than, or is less than about: 0.1, 0.2, 0.3, 0.4, 0.5, 0.6, 0.7, 0.8, 0.9, 1, 1.25, 1.5, 1.75, 2.0, 2.25, 2.5, 2.75 or 3.0 mg/L, or at any concentration within a range defined by any two of the aforementioned concentrations, for example, 0.1 to 3 mg/L, 0.5 to 2.0 mg/L, 0.5 to 1.5 mg/L, 0.3 to 2.5 mg/L, or 0.5 to 1.75 mg/L; or 0.1, 0.2, 0.3, 0.4, 0.5, 0.6, 0.7, 0.8, 0.9, or 1 mg/L, or any concentration within a range defined by any two of the aforementioned concentrations, for example, 0.1 to 1 mg/L, 0.1 to 0.5 mg/L, 0.5 to 1 mg/L, 0.3 to 0.7 mg/L, or 0.4 to 0.6 mg/L.

In some embodiments, provided herein are compositions comprising mature liver organoids. In some embodiments, provided herein are compositions comprising mature liver organoids that exhibit luminal projections that resemble bile canaliculi, and/or a structure having a single lumen and generally a spherical shape. In some embodiments, provided herein are compositions comprising mature liver organoids that were produced through contact with a exposure to exogenous bilirubin.

Also provided herein, in some embodiments, are compositions comprising posterior foregut cells and/or posterior foregut endoderm cells, liver organoids and/or mature liver organoids that have been engineered to comprise a functional L-gulonolactone oxidase (GULO) protein and/or a gene or mRNA, or both, that encodes for the functional GULO protein, wherein the posterior foregut cells and/or posterior foregut endoderm cells, liver organoids and/or mature liver organoids are able to synthesize ascorbate. In some embodiments, provided herein are compositions comprising posterior foregut cells and/or posterior foregut endoderm cells, liver organoids and/or mature liver organoids engineered to express functional GULO protein, wherein the functional GULO protein is murine GULO (mGULO). In some embodiments, a gene that encodes for a functional GULO protein is conditionally expressed. In some embodiments, a gene that encodes for a functional GULO protein is constitutively expressed. In some embodiments, a gene that encodes for a functional GULO protein is conditionally expressed using a tetracycline inducible system.

In some embodiments, provided herein are compositions comprising posterior foregut cells and/or posterior foregut endoderm cells, liver organoids and/or mature liver organoids that are engineered to comprise a gene that encodes for a functional GULO protein using CRISPR mediated knock-in. In some embodiments, provided herein are compositions comprising posterior foregut cells and/or posterior foregut endoderm cells, liver organoids and/or mature liver organoids comprising a functional GULO encoding gene or mRNA, or both, that encodes for a functional GULO protein, wherein the functional gene was introduced to the posterior foregut cells and/or posterior foregut endoderm cells, liver organoids, mature liver organoids, and/or precursor cells by transfection. In some embodiments, provided herein are compositions comprising posterior foregut cells and/or posterior foregut endoderm cells, liver organoids and/or mature liver organoids that are engineered to comprise a gene that encodes for a functional GULO protein using adenovirus mediated gene transfection. In some embodiments, provided herein are compositions comprising posterior foregut cells and/or posterior foregut endoderm cells, liver organoids and/or mature liver organoids that are engineered to comprise a gene that encodes for a functional GULO protein using adeno-associated virus mediated gene transfection.

In some embodiments, compositions provided herein comprise liver organoids and/or mature liver organoids comprising a functional GULO protein, wherein said liver organoids and/or mature liver organoids express increased levels of NRF2 relative to liver organoids and/or mature liver organoids that do not comprise a functional GULO protein. In some embodiments, compositions provided herein comprise liver organoids and/or mature liver organoids comprising a functional GULO protein, wherein the liver organoids and/or mature liver organoids express reduced levels of IL1B, IL6, or TNFa, or any combination thereof, relative to liver organoids and/or mature liver organoids that do not comprise a functional GULO protein. In some embodiments, liver organoids and/or mature liver organoids comprising a functional GULO protein exhibit reduced caspase-3 activity relative to liver organoids and/or mature liver organoids that do not comprise a functional GULO protein. In some embodiments, liver organoids and/or mature liver organoids comprising a functional GULO protein express increased levels of ALB relative to liver organoids and/or mature liver organoids that do not comprise the functional GULO protein. In some embodiments, liver organoids and/or mature liver organoids comprising a functional GULO protein resemble periportal liver tissue and/or express periportal liver markers. In some embodiments, periportal liver markers comprise or consist of FAH, ALB, PAH, CPS1, HGD, or any combination thereof. In some embodiments, liver organoids and/or mature liver organoids comprising a functional GULO protein exhibit increased CYP3A4 and/or CYP1A2 protein levels and/or enzymatic activity relative to liver organoids and/or mature liver organoids that do not comprise a functional GULO protein. In some embodiments, liver organoids and/or mature liver organoids comprising a functional GULO protein exhibit increased bilirubin conjugation activity relative to liver organoids and/or mature liver organoids that do not comprise a functional GULO protein. In some embodiments, liver organoids and/or mature liver organoids comprising a functional GULO protein exhibit increased viability in culture relative to liver organoids and/or mature liver organoids that do not comprise a functional GULO protein. In some embodiments, liver organoids and/or mature liver organoids have been differentiated from pluripotent stem cells comprising a functional GULO protein and/or a gene or mRNA, or both, that encodes for the functional GULO protein, whereby the pluripotent stem cells are able to synthesize ascorbate.

### Kits

In some embodiments, also disclosed herein are kits providing means for performing any of the methods described herein. In some embodiments, also disclosed herein are kits comprising any of the compositions or means of producing the compositions described herein.

In some embodiments, the kits of the disclosure can include one or more FGF signaling pathway activator, one or more Wnt signaling pathway activator, one or more RA signaling pathway activator, and one or more BMP signaling pathway inhibitor. In some embodiments, the kits of the disclosure can further include iPSCs, PSCs, and/or posterior foregut cells and/or posterior foregut endoderm cells. In some embodiments, the kits of the disclosure can further include a cell culture medium, such as a hepatocyte culture medium.

In some embodiments, a kit can be prepared from readily available components and reagents. For example, such kits can comprise any one or more of the following components and/or reagents: enzymes, reaction tubes, buffers, detergent, primers, probes, antibodies, cell culture media, differentiation induction reagents, amino acid mixtures/supplements, engineered constructs and/or polynucleotides, transcription induction agents, bilirubin, ascorbic acid, ascorbate, retinoic acid pathway activators, corticosteroids, cMET tyrosine kinase receptor agonists, IL-6 family cytokines, TGF-b pathway inhibitors, FGF pathway activators, Wnt pathway activators, VEGF pathway activators, ROCK inhibitors, organoids, and/or cells. In some embodiments, components and reagents may be packaged together in any combination, and/or may be packaged individually. In some embodiments, kits may include components and reagents concentrated above the working concentrations disclosed herein, or at the working concentrations provided herein. In some embodiments, individual components may also be provided in a kit in concentrated amounts; in some aspects, a component is provided individually in the same concentration as it would be in a solution with other components. In some embodiments, concentrations of components may be provided as 1×, 2×, 5×, 10×, or 20× or more. In some embodiments, a kit may comprise components, which may be individually packaged or placed in a container, such as a tube, bottle, vial, syringe, or other suitable container means.

In some embodiments, a kit is housed in a container. Kits may further comprise instructions for using the kit for assessing expression and/or differentiation of cells. Agents in a kit for measuring expression and/or determining differentiation may comprise a plurality of PCR probes and/or primers for qRT-PCR and/or a plurality of antibody or fragments thereof for assessing expression of biomarkers appropriate for classifying cell states.

In some embodiments, kits are created using and comply with good manufacturing practice (GMP).

Having described the embodiments in detail, it will be apparent that modifications, variations, and equivalent embodiments are possible without departing from the scope of the embodiments defined in the appended claims. Furthermore, it should be appreciated that all examples in the present disclosure are provided as non-limiting examples.

### EXAMPLES

### Example 1. Characterization of Sympathetic Neurons, or Sympathetic Nerves, in Human Liver Organoids

Liver organoids were generated from human iPSCs using modified versions of previously disclosed methods (“conventional” HLO methods). Foregut spheroids were generated from embryonic endoderm cells differentiated from human iPSCs and embedded in Matrigel basement membrane matrix. Then, the Matrigel-embedded foregut spheroids were exposed to retinoic acid for 4 days and cultured in a liver maturation-inducing medium to generate liver organoids. The human liver organoids spontaneously generated sympathetic neurons, or sympathetic nerves, that co-expressed the neural marker class III beta tubulin (TUBB3) and the sympathetic marker tyrosine hydroxylase (TH) (FIG. 1).

In contrast, no neurons or nerves were detected in human liver organoids generated by conventional methods, when used as a control for the human liver organoids generated by the modified methods as described herein. The ability to generate neuronal cells in human liver organoids was remarkable, given that there has been no previous documentation of any TUBB3 and/or TH expression in any human liver organoid prepared by any previously published or conventional methods. Thus, any neurons present in human liver organoids generated by conventional methods would be less than 1% of the total cells of the human liver organoid, at or below the lower limits of detection.

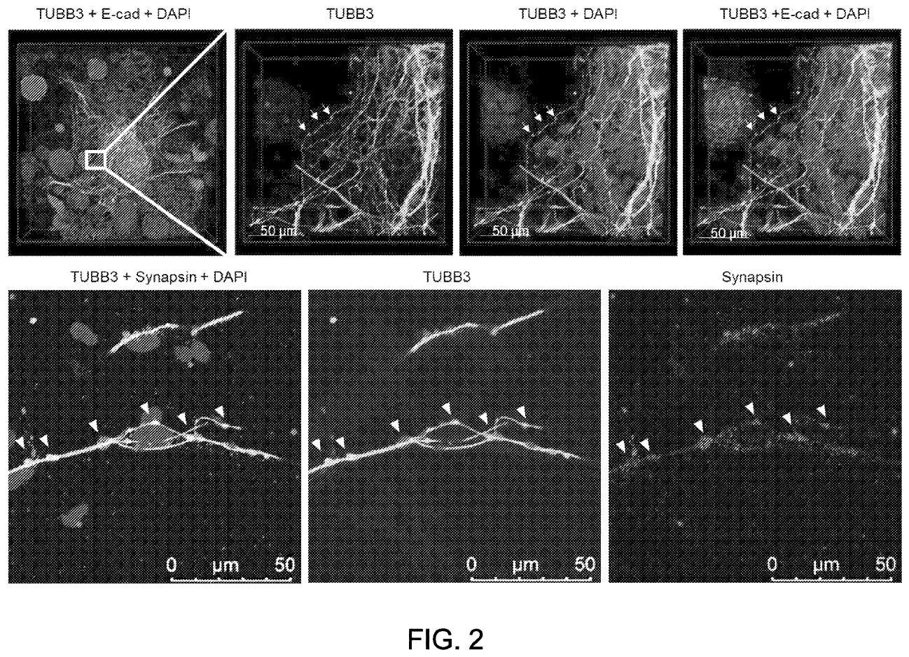

In order to clarify whether the sympathetic neurons, or sympathetic nerves, established in human liver organoids have the ability to produce noradrenaline, a neurotransmitter of the sympathetic nervous system, it was tested whether the neurotransmitter-producing varicosity region and vesicles that store and secrete neurotransmitters were present in the sympathetic neurons, or sympathetic nerves, of the human liver organoids. Whole-mount immunostaining of human liver organoids with synapsin (SYN), a membrane protein marker of secretory vesicles, and TUBB3, a neural marker, revealed a hump-shaped varicosity region in the sympathetic neurons, or sympathetic nerves, in the human liver organoids, and the presence of the expression of SYN in a speckle-like pattern characteristic of secretory vesicles, was detected in the region (FIG. 2; arrows indicate varicosity sites).

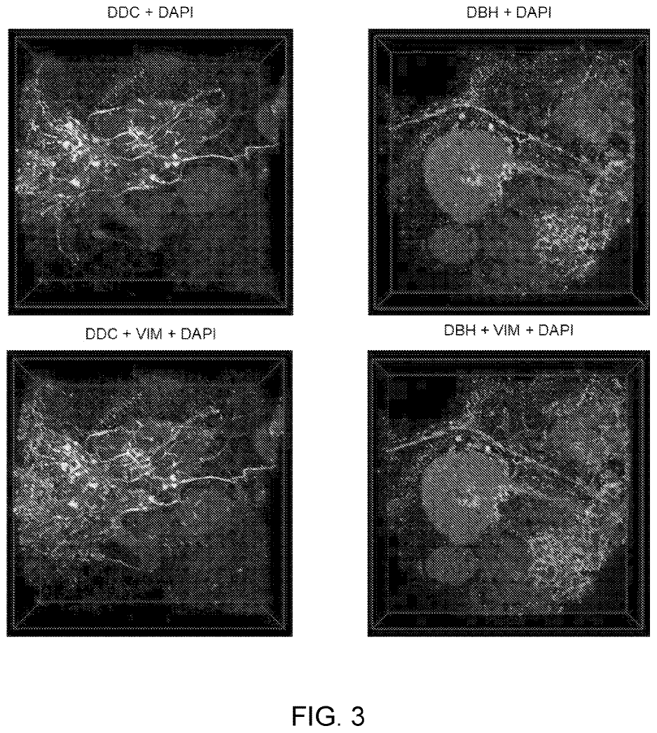

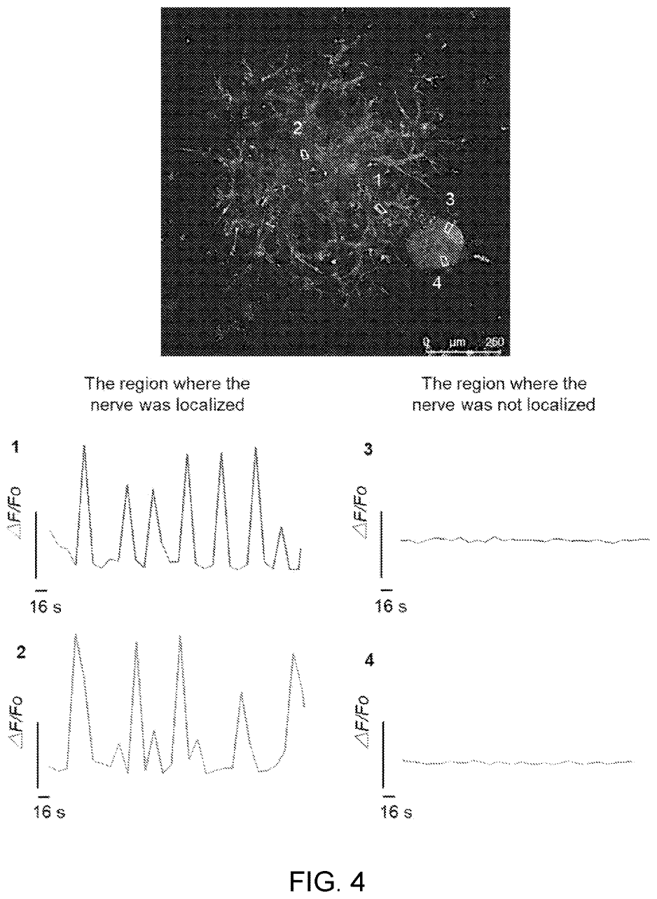

The expression of dopa decarboxylase (DDC) and dopamine β-hydroxylase (DBH), which are noradrenaline synthesizing enzymes, was also detected in human liver organoids, indicated that sympathetic neurons, or sympathetic nerves, in human liver organoids have the ability to produce noradrenaline (FIG. 3). To clarify whether the sympathetic neurons, or sympathetic nerves, in the human liver organoids are spontaneously activated, liver organoids were generated from iPSCs transfected with the fluorescent calcium sensor GCaMP gene (GCaMP-iPSCs), and the neural activity was evaluated by the fluorescence intensity in the liver organoids. The fluorescence in the entire liver organoids were monitored and fluorescence was only observed in the region where the nerves were localized, where the fluorescence activity pattern showed a synchronization specific to nerves (FIG. 4). These results indicate that sympathetic neurons, or sympathetic nerves, that produce noradrenaline and spontaneous neural activity can be formed in human liver organoids prepared according to the modified methods described herein.

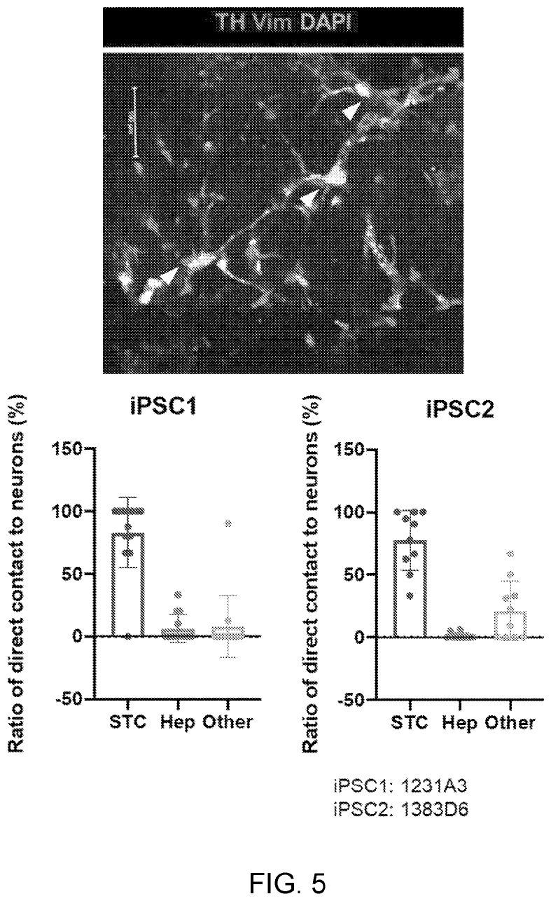

The target cells of the sympathetic neurons, or sympathetic nerves, in the liver organoids were then characterized, and it was determined whether those nerves contribute to the activity of noradrenaline receptor signaling in these cells. In addition to TUBB3, hepatic stellate cells and hepatocytes, which are the major constituent cells of liver organoids, were stained with vimentin (VIM) and E-cadherin (E-cad), respectively, to identify cells to which the sympathetic varicosity region is localized adjacent in the human liver organoids. Staining of liver organoids revealed that approximately 80% of the sympathetic varicosity region was localized adjacent to VIM-positive hepatic stellate cells (FIG. 5; arrows indicate the TH cells localized adjacent to VIM-positive cells).

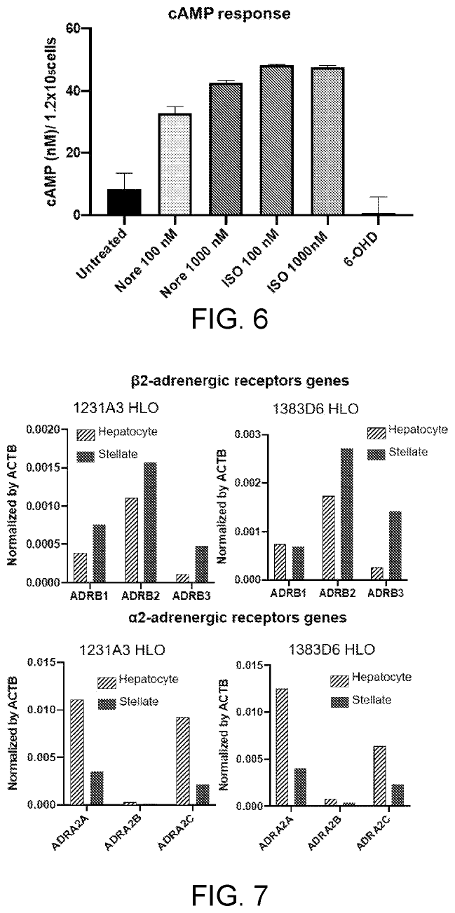

Next, it was clarified whether the sympathetic neurons, or sympathetic nerves, in human liver organoids contribute to the activity of adrenergic receptor signaling in hepatic stellate cells. The sympathetic neurons, or sympathetic nerves, in human liver organoids were ablated by 6-hydroxydopamine (6-OHDA), a sympathetic-specific toxin, and the intracellular levels of cyclic AMP (CAMP), a second messenger of adrenergic receptors in human liver organoids, were measured. The results showed that the concentration of CAMP was significantly decreased in 6-OHDA-treated organoids compared to untreated organoids (FIG. 6).

On the other hand, exposure to noradrenaline or the β-adrenergic receptor agonist isoproterenol (ISO) increased the concentration of cAMP (FIG. 6). Since CAMP is a second messenger for β among the adrenergic receptor subtypes (α1, α2, and B), and since hepatic stellate cells in human liver organoids expressed higher levels of β-adrenergic receptor genes (ADRB1, ADRB2, and ADRB3) and lower levels of α2-adrenergic receptor genes (ADRA2A, ADRA2B, and ADRA2C) than other cells (FIG. 7), sympathetic neurons, or sympathetic nerves, in the organoids can activate β-adrenergic receptor signaling in hepatic stellate cells via noradrenaline.

Taken together, these results demonstrate that human liver organoids prepared according to the modified process developed as described herein, contain sympathetic neurons, or sympathetic nerves, that target hepatic stellate cells, the same as human intrahepatic sympathetic neurons, or sympathetic nerves, and are a useful liver model for examining the effects of human intrahepatic sympathetic neurons, or sympathetic nerves, on fatty liver. In addition, it was revealed that sympathetic neurons, or sympathetic nerves, in human liver organoids can activate β-adrenergic receptors in hepatic stellate cells via noradrenaline.

### Example 2. Verification of the Contribution of Sympathetic Neurons, or Sympathetic Nerves, in Human Liver Organoids to Lipid Accumulation and Inflammation

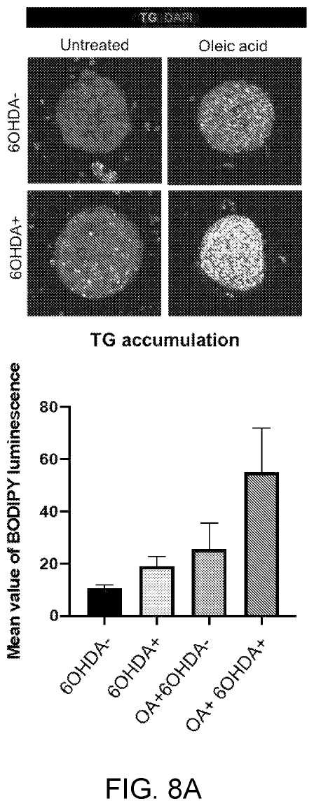

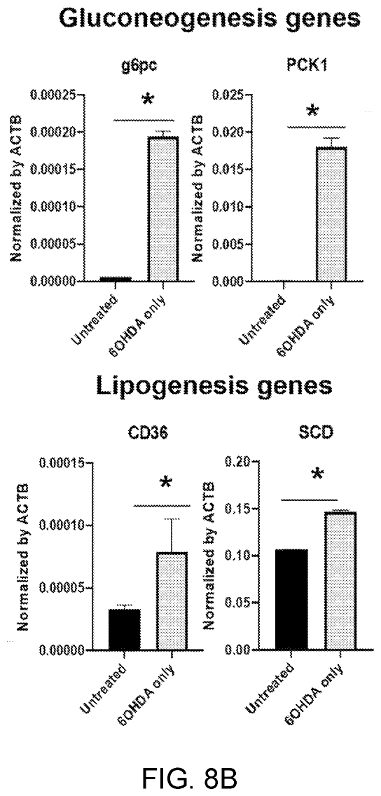

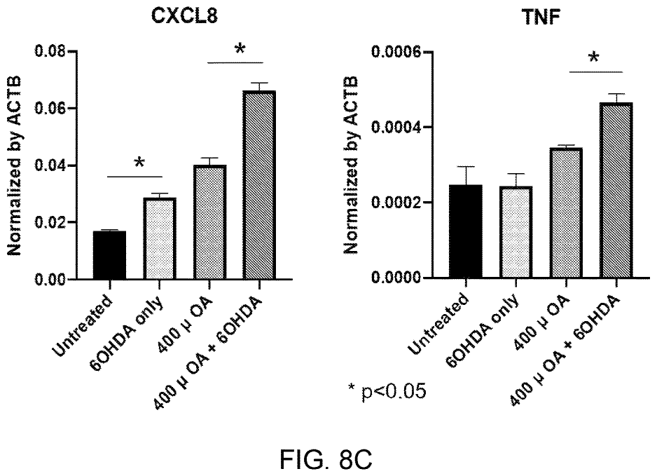

Next, it was determined whether sympathetic neurons, or sympathetic nerves, in human liver organoids contribute to lipid accumulation in hepatocytes. After treatment with 6-OHDA, triglyceride (TG) in hepatocytes of the organoids were measured by live cell imaging using BODIPY 493/503, a fluorescent probe for TG, and the accumulation of TG in hepatocytes of 6-OHDA-treated and untreated organoids was compared. TG accumulation in hepatocytes of 6-OHDA-treated organoids was about twice that of untreated organoids (FIG. 8A). In addition, TG accumulation in hepatocytes of organoids treated with 6-OHDA and then exposed to oleic acid (OA) for 3 days was about 3-fold higher than that of OA-only treated organoids (FIG. 8A). These results demonstrate that sympathetic neurons, or sympathetic nerves, in human liver organoids inhibit TG accumulation in hepatocytes.

As quantified by RT-qPCR, 6-OHDA treated organoids exhibited increased expression of glucose-6-phosphatase catalytic subunit (G6PC) and phosphoenolpyruvate carboxykinase 1 (PCK1), which are associated with gluconeogenesis; and CD36 and stearoyl-CoA desaturase (SCD), which are associated with lipogenesis (FIG. 8B). Furthermore, under steatosis conditions (treated with oleic acid), organoids additionally treated with 6-OHDA expressed increased expression of the cytokines interleukin 8 (CXCL8; IL8) and tumor necrosis factor (TNF) relative to those not treated with 6-OHDA (FIG. 8C). This suggests that sympathetic neurons, or sympathetic nerves, in human liver organoids are involved in inflammation suppression.

### Example 3. Characterization of Gene Expression in Human Liver Organoids

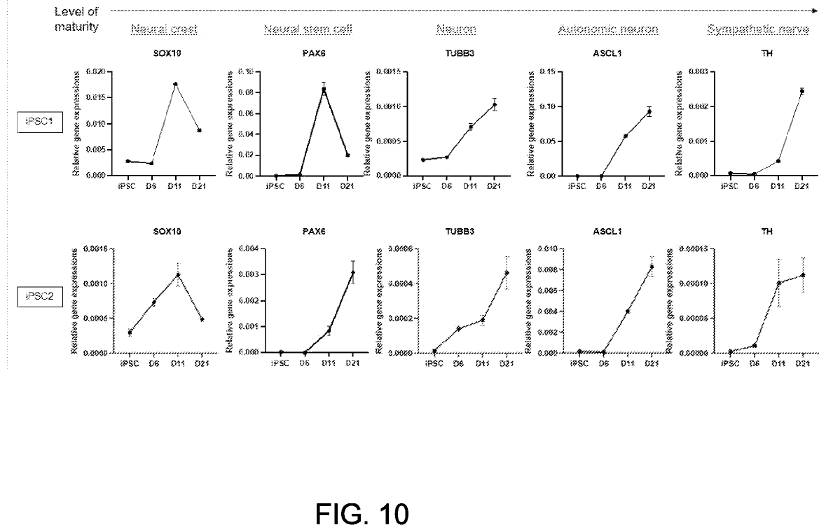

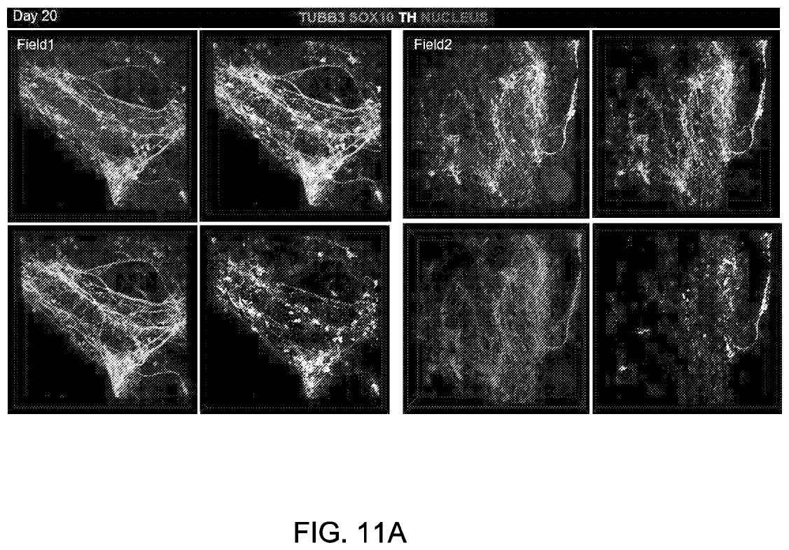

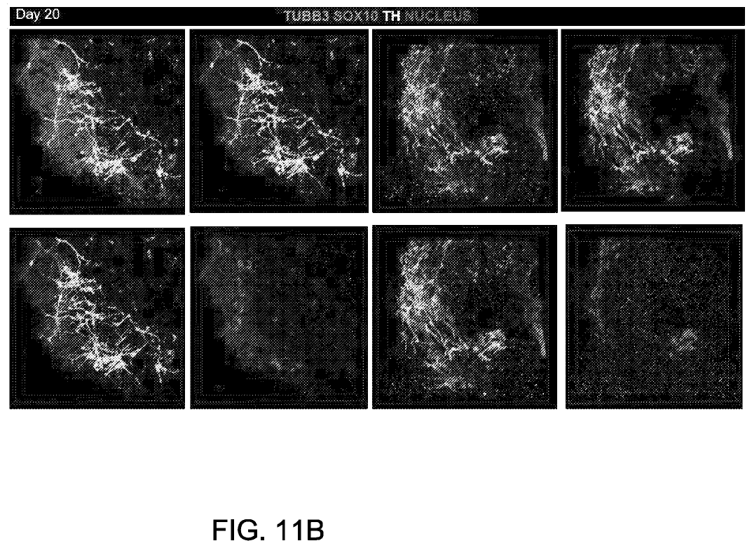

During the process of human liver organoid production from iPS cells, real-time PCR quantification was performed on days 0 (IPSC), 5 (D5), 11 (D11) and 21 (D21). Real-time PCR indicated that the expression of neural crest and neural stem cell markers SOX10 (neural crest cell marker) and PAX6 (neural stem cell marker) decreased during the process of HLO production from iPS cells. However, expression of markers related to neurons, TUBB3 (Neuron marker), ASCL1 (Autonomic neuron marker), and TH (Sympathetic nerve marker), increased during the process (FIG. 10). Whole mount immunostaining of HLOs (day 20 after iPSC differentiation and liver induction) showed that SOX10+ neural crest-like cells were always present around TH+/TUBB3+ sympathetic-like nerves in HLOs (FIG. 11A). In contrast, TH−/TUBB3+ non-sympathetic-like nerves were produced when the number of SOX10+ neural crest-like cells was low (FIG. 11B). The immunostaining and real-time PCR results demonstrate that SOX10+ neural crest-like cells produce TH+ sympathetic-like nerves in HLOs.

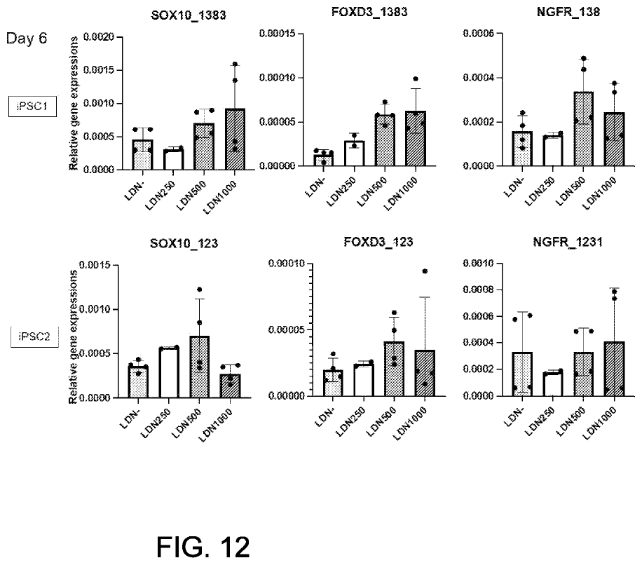

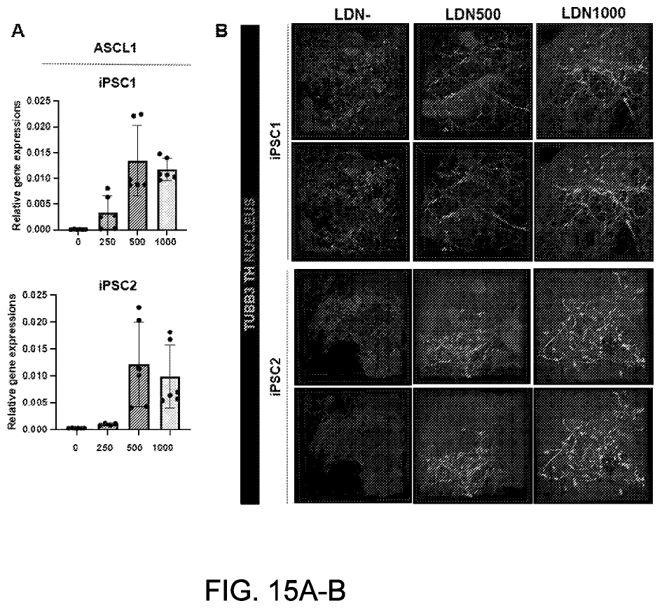

Foregut spheroids were treated with 250 nM, 500 nM, or 1000 nM of the BMP inhibitor LDN193189 for 72 hours from day 3 (D3) to day 6 (D6) of differentiation, and real-time PCR quantification was performed on D6. Real-time PCR indicated that BMP inhibitor LDN193189 increased expression of neural crest markers (SOX10, FOXD3, and NGFR) in foregut spheroids. (FIG. 12). These results demonstrate that inhibition of BMP signaling increases neural crest cells in foregut spheroids. Real-time PCR indicated that expression of autonomic marker ASCL1 was increased in HLOs on day 20 of differentiation induction. (FIG. 15A). Immunostaining of HLOs on day 20 of differentiation induction showed that with the addition of BMP inhibitor LDN-193189, TH+/TUBB+ sympathetic-like cells were generated in HLOs; and without LDN-193189 treatment, sympathetic-like cells were quite rare or were not generated (FIG. 15B). These results indicate that BMP inhibition is an important factor for the creation of sympathetic-like cells in HLOs and that the sympathetic-like cells in HLOs are derived from neural crest-like cells.

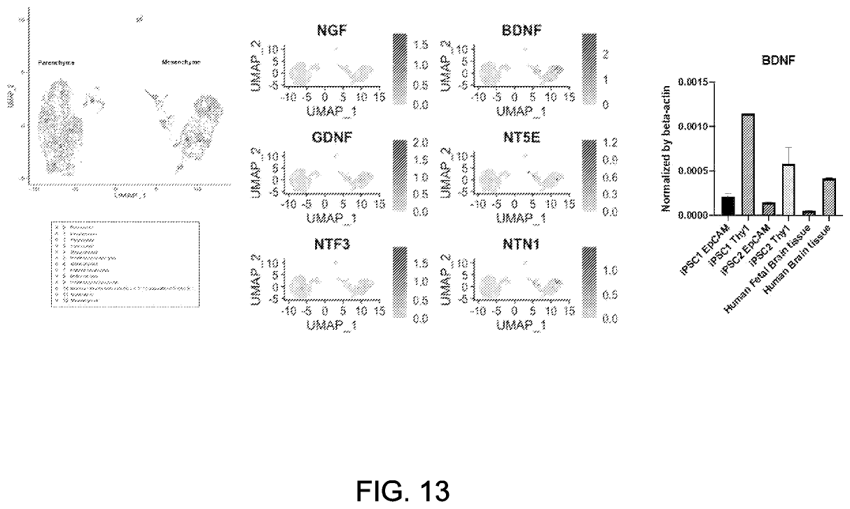

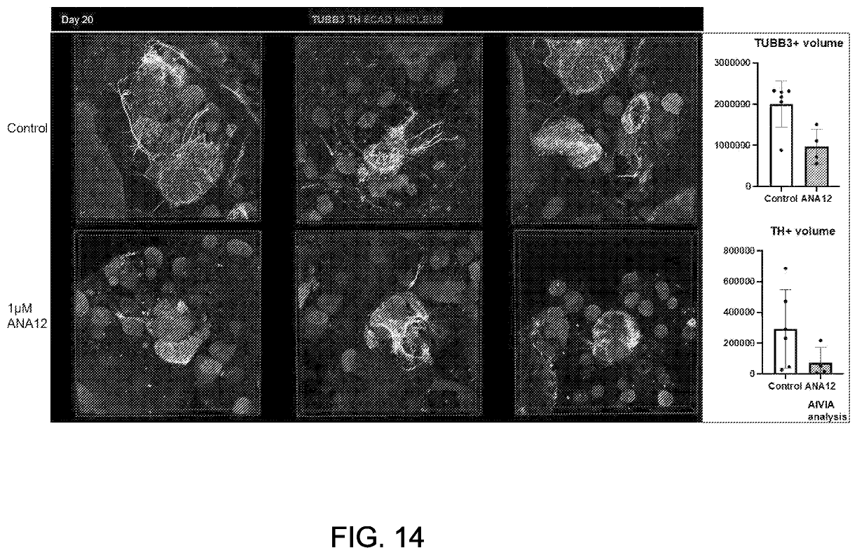

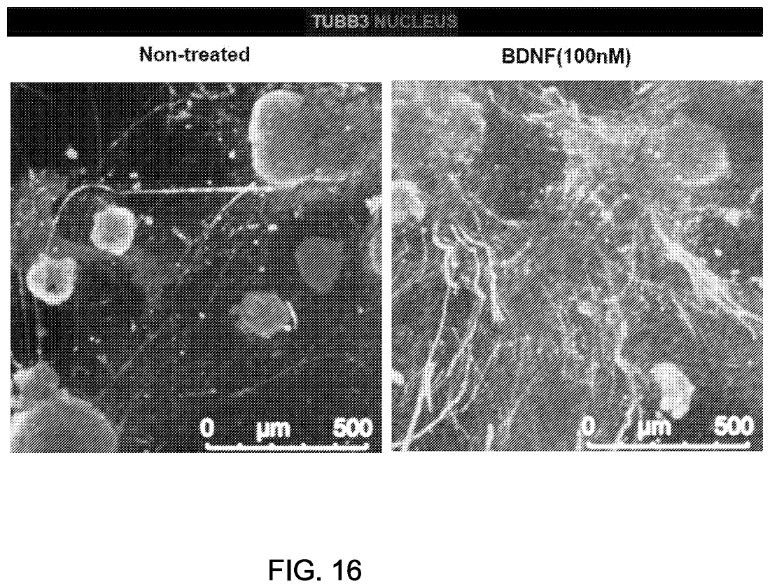

Single cell RNA sequencing (scRNAseq) and real-time PCR quantification of gene expression were performed in day 20 (D20) liver organoids, and it was noted that the neuron attractive factor BDNF was highly expressed in hepatic stellate-like cells of the liver organoids. (FIG. 13). HLOs were treated with and without 1 μM BDNF inhibitor ANA12 for 5 days from day 15 (D15) to day 20 (D20) of differentiation induction. Immunostaining and cell volume analysis (μm³) of TUBB3+ and TH+ ANA12-treated human liver organoids (day 20 after iPSC differentiation and liver induction) indicated that ANA12 treatment on day 15 suppressed TH+ nerve production and elongation of nerves in HLOs. (FIG. 14). These results demonstrate that BDNF secreted from hepatic stellate-like cells increase TH+ sympathetic-like nerves production in HLOs. HLOs were treated with and without 100 nM of BDNF for 13 days from day 15 (D15) to day 28 (D28) of differentiation induction. Immunostaining of BDNF-treated human liver organoids indicated that when BDNF was added, nerves tend to increase and elongate (FIG. 16).

### Example 4. Materials and Methods

Differentiation of Human iPSCs into Posterior Foregut Spheroids:

Colonies of iPSCs were detached with Accutase (Thermo Fisher Scientific), and 200,000 cells were seeded on Matrigel or laminin-511 (e.g., iMatrix) coated tissue culture plate (VWR). The medium was changed to RPMI 1640 medium containing 100 ng/ml Activin A (R&D Systems) and 50 ng/ml bone morphogenetic protein 4 (BMP4; R&D Systems) at day 1; 100 ng/mL Activin A and 0.2% fetal calf serum (FCS; Thermo Fisher Scientific) at day 2; and 100 ng/ml Activin A and 2% FCS at day 3. On Day 4-5, cells were cultured in Advanced DMEM/12 with B27, N2, 10 mM HEPES, and 2 mM L-glutamine containing 500 ng/mL fibroblast growth factor 4 (FGF4; R&D Systems), 2 μM CHIR99021 (Stemgent), and 250 nM LDN-193189. On Day 6, cells were cultured with 500 ng/mL FGF4, 2 μM CHIR99021, 250 nM LDN-193189, and 2 μM retinoic acid. Cells were maintained at 37° C. at 5% CO₂ with 95% air and the medium was replaced every day. Posterior foregut spheroids appeared on the plate at Day 6 of differentiation.

Embedding of Posterior Foregut Spheroids into Matrigel Drop:

At Day 6, spheroids and attached cells were vigorously pipetted to be delaminated from the dish. They were centrifuged at 800 rpm for 3 min, embedded in three drops (70 μL) of 100% Matrigel from one well of posterior foregut spheroids, and cultured in Advanced DMEM/F12 with B27, N2, 10 mM HEPES, 2 mM L-glutamine, and 2 mM retinoic acid (RA; Sigma) for 4 days. In order to induce liver organoids with sympathetic neurons, or sympathetic nerves, the seeding density of the cells in a single drop was increased by about 85%-110% relative to previous methods (e.g., approximately 7.4×10⁵ cells/drop, compared to 4.0×10⁵ cells/drop).

### Human Liver Organoid (HLO) Induction:

After RA treatment, the medium was switched to Hepatocyte Culture Medium (HCM; Lonza) with 10 ng/mL hepatocyte growth factor (HGF; PeproTech), 0.1 mM Dexamethasone (Dex; Sigma) and 20 ng/mL Oncostatin M (OSM; R&D Systems). Cultures for HLO induction were maintained at 37° C. in 5% CO₂ and the medium was replaced every 3 days. To analyze HLO (Day 20-30), organoids were isolated from Matrigel by scratching and pipetting.

### Example 5. Comparison of HLO Generation Methods

The methods provided herein to produce liver organoids with intrahepatic sympathetic neurons, or sympathetic nerves, are modified from Ouchi et al. “Modeling Steatohepatitis in Humans with Pluripotent Stem Cell-Derived Organoids” Cell Metabolism 30, 374-384 and PCT Publication WO 2018/085615, each of which is hereby expressly incorporated by reference in its entirety. Important differences from these previous protocols, which were found to lead to formation of the sympathetic neurons, or sympathetic nerves, in HLOs, include the inhibition of the BMP signaling pathway (for example, by addition of the BMP inhibitor LDN-193189) during the posterior foregut spheroid culturing steps, and the use of increased seeding density during basement membrane culture. One skilled in the art will recognize that alternative BMP inhibitors, or other means of effecting BMP signaling inhibition, can be used and are expected to provide a similar effect.

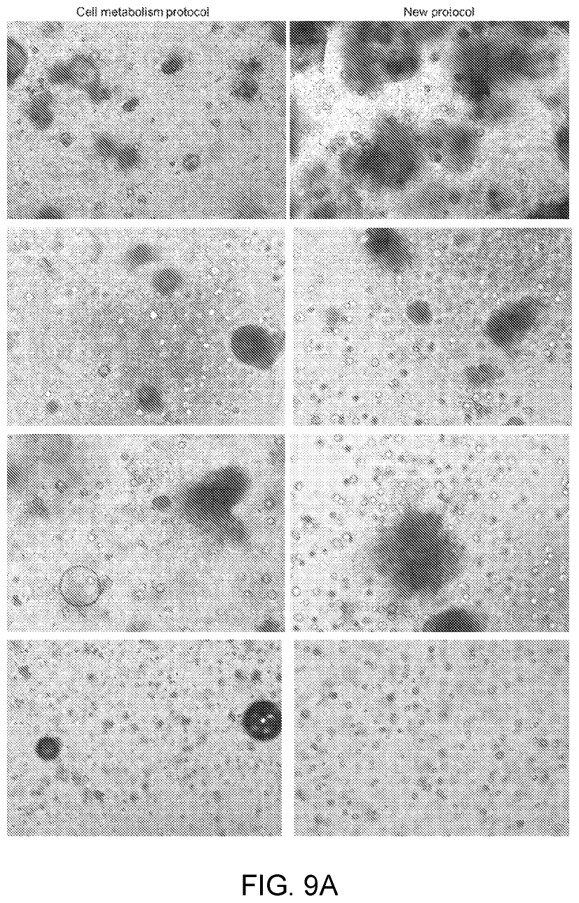

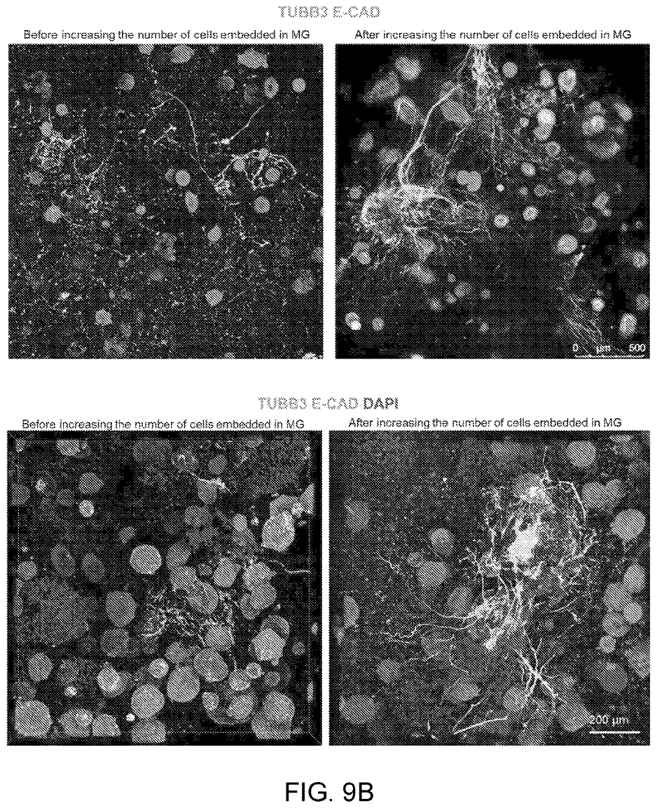

Liver organoids were produced according to the methods described herein and in Ouchi et al. By bright field microscopy, these liver organoids are morphologically comparable (FIG. 9A). However, when these liver organoids are stained for TUBB3 (neurons) and E-cad (hepatocytes), the liver organoids produced from higher seed density Matrigel drops exhibit greater numbers of TUBB3 positive neurons (FIG. 9B).

### Example 6. Investigation of the Role of Sympathetic Neurons, or Sympathetic Nerves, in HLO on Hepatic Lipid Accumulation, Inflammation, and Fibrosis

Fluorescent immunostaining of Collagen Type I Alpha 1 chain is performed using 6-OHDA treated HLO sections to evaluate fibrosis. Similar experiments are performed with the addition of α- and β-adrenaline receptor antagonists, prazosin and propranolol, and the selective 5-HT and noradrenaline re-uptake inhibitors venlafaxine and levomilnacipran instead of 6-OHDA. Furthermore, shRNA-mediated knockdown and CRISPR/Cas9-mediated knockout of adrenaline receptors are performed. Levels of the receptor protein expression are detected by whole mount staining.

The pLV [Exp]-{rTH promoter}> {hM3D (Gq) (ns)}: T2A: mCherry or pLV [Exp]-SYN1> {hM3D (Gq) (ns)}: T2A: mCherry constructs are introduced into GCaMP4 iPSCs using lentivirus vectors. These iPSCs express neuron-specific (rTH promotor or hSyn promoter) designer receptors exclusively activated by designer drugs (DREADDs) that are excitatory (hM3D). HLO derived from hM3D-induced iPSCs are cultured with DREADD agonist clozapine N-oxide (CNO) for inducing hM3D-driven neuronal stimulation. The imaging of neuronal activity of those HLO using calcium indicators are employed using confocal microscopy. Additionally, noradrenaline release and cAMP levels are determined by enzyme-linked immunosorbent assay (ELISA). These genetically modified liver organoids can also be used for the adrenaline receptor experiments provided above.

pLenti-TH-hChR2 (H134R)-EYFP-WPRE or pLenti-Synapsin-hChR2 (H134R)-EYFP-WPRE constructs are introduced into GCaMP4 iPSCs using lentivirus vectors. These iPSCs express neuron-specific (rTH promoter or hSyn promoter) channelrhodopsin-2 (ChR2). HLO derived from hChR2-induced iPSCs are cultured with continuous illumination of blue light for inducing hChR2-driven neuronal stimulation. The imaging of neuronal activity of these HLO are performed using calcium indicators and multielectrode array (MEA). For MEA, HLO are plated and cultured onto an MEA substrate. These genetically modified liver organoids can also be used for the adrenaline receptor and/or DREADD experiments provided above.

A single slice (350-599 μm) of HLO is transferred and secured with a slice anchor in a large volume bath for confocal microscopy. Slices are continuously perfused at a rate of 2.5-3 mL/min with a recording solution. Whole-cell current and voltage-clamp recordings are conducted at 32-33° C. with an Axopatch-200B amplifier using 5-8 MOhm glass electrodes filled with an internal solution. These cell samples can also be used for the adrenaline receptor and/or DREADD experiments provided above.

### Example 7. Culture Conditions Responsible for Spontaneous Generation of Sympathetic Neurons, or Sympathetic Nerves, in iPS Cell-Derived HLOs Via Neural Crest Cells

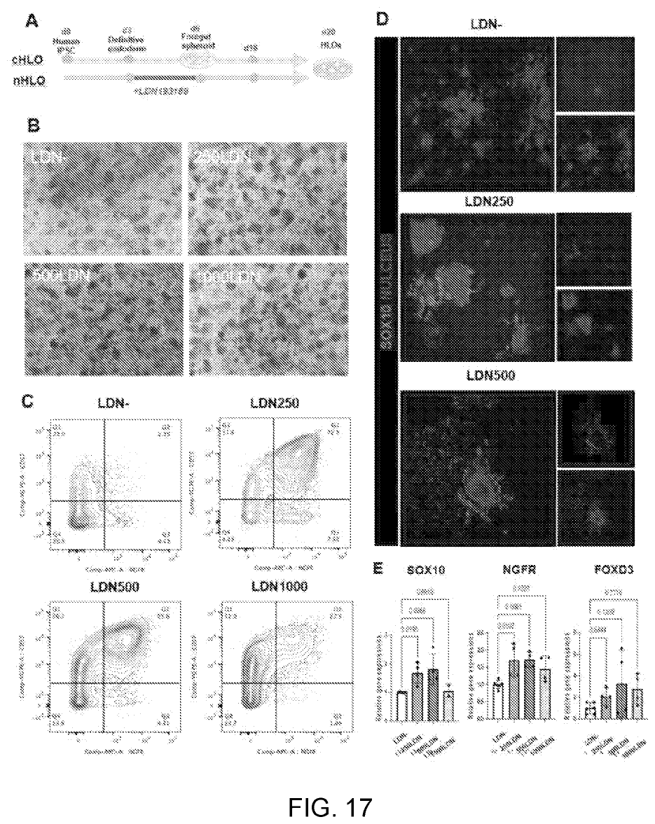

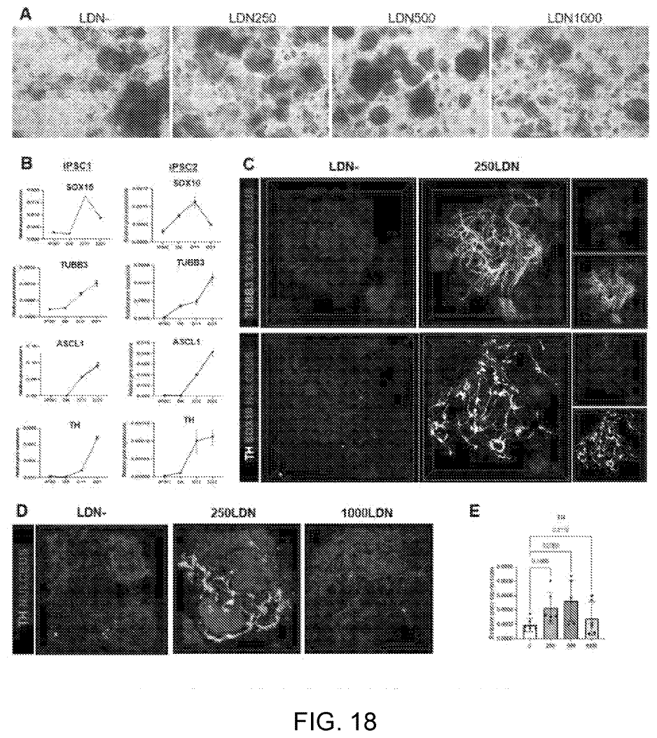

In the organoid conditions, the production of neural crest cells, TH/TUBB+ sympathetic neurons and liver production under LDN193189 250 nM, 500 nM, and 1000 nM (added on days 3 to 6 of iPS cell differentiation induction) were compared. The results showed that 250 nM and 500 nM LDN were the optimal concentrations for induction of neural crest cells and TH/TUBB+ sympathetic neurons. At 1000 nM, the production of neural crest cells was reduced and TH/TUBB+ sympathetic neurons was also reduced, demonstrating that the sympathetic innervation of HLO is dependent on neural crest cells (FIG. 17A-E and FIG. 18A-E).

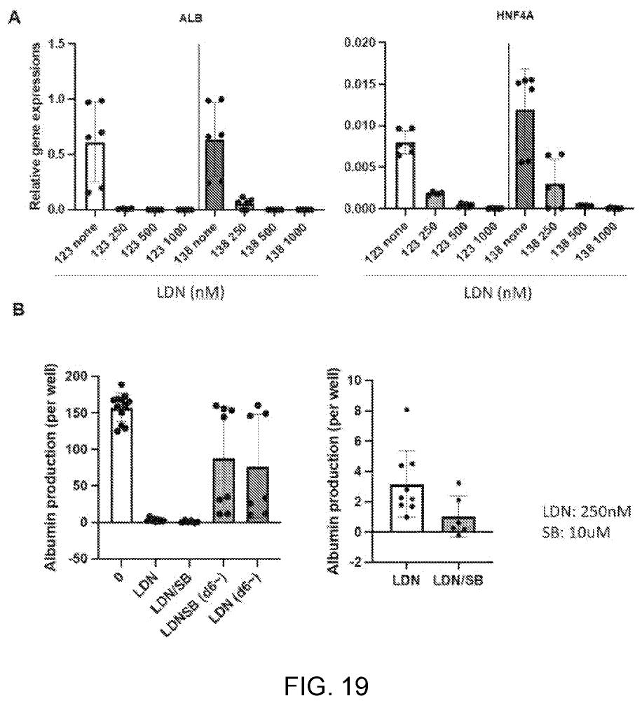

As gene expression levels of the liver markers albumin (ALB) and HNF4A have been shown to decrease in a LDN concentration-dependent manner, sympathetic innervation was induced at the lowest concentration of 250 nM as a comparison condition. In addition, compared to existing HLOs, albumin secretion is 30-40-fold lower at 250 nM LDN (FIG. 19A-B).

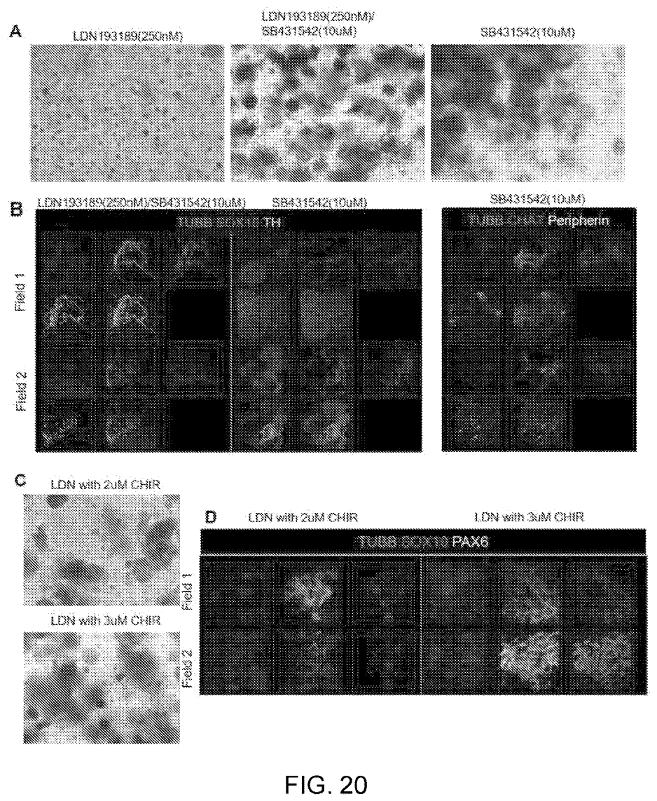

When SB431542 was administered in addition to LDN, extensive TUBB+/TH+ sympathetic innervation was formed around day 15, when LDN alone had not yet developed innervation (too many cells, so sampling was required on day 15). However, albumin secretion was minimal (FIG. 19A-B). Under SB alone, TUBB3-positive cells were observed, but no TH signal was observed. In contrast, expression of the parasympathetic marker CHAT was detected in some cells. When 3 μM CHIR was added instead of 2 μM CHIR, more TUBB3+ and SOX10 neural crest cells were found compared to the LDN alone group (reproducible in separate batches) (FIG. 20A-D).

Changing the timing of LDN addition from Day 3-6 to Day 6-10 improves induction to the liver. This had the result of reducing albumin secretion by a third compared to conventional HLOs (FIG. 19A-B), although the frequency of sympathetic generation varies between batches.

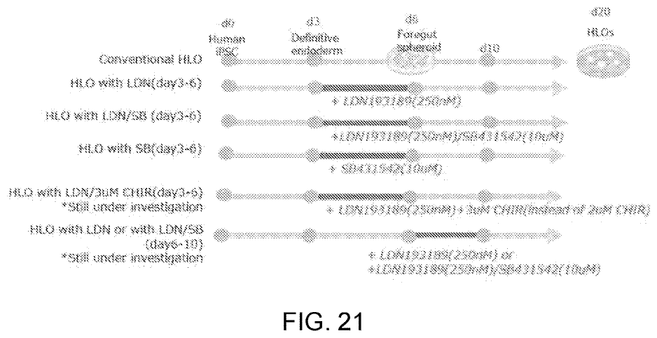

Various aspects of appropriate culture conditions and methods sufficient for spontaneous generation of sympathetic neurons, or sympathetic nerves, in iPSC-derived HLOs were then evaluated, as shown in FIG. 21, namely:
Conventional HLO culturing method
HLO culture with BMP inhibition at definitive endoderm stage
HLO culture with BMP inhibition+SB at definitive endoderm stage
HLO culture with SB
HLO culture with BMP inhibition+CHIR at definitive endoderm stage
HLO culture with BMP inhibition+CHIR at foregut spheroid stage

Human iPSCs were differentiated according to previously described conventional/standard HLO culturing methodology. It was found that with the conventional HLO culturing method, detection of neural crest cells and TH+/TUBB3+ sympathetic neurons, or sympathetic nerves, is quite rare, and any neural cells, if present at all, are less than 1%.

Next, human iPSCs were differentiated as with conventional HLO culture, but with the addition of 250 nM of the BMP inhibitor LDN193189 from days 3-6, i.e. when differentiating the definitive endoderm into foregut spheroids. A high frequency of neural crest cells and TUBB3/TH+ neurons were detected; gene expression of liver markers ALB and HNF4A was found to be reduced, as was ALB secretion.

Next, human iPSCs were differentiated as with conventional HLO culture, but with the addition of 250 nM of the BMP inhibitor LDN193189 and 10 μM SB431542 from days 3-6, i.e. when differentiating the definitive endoderm into foregut spheroids. Neural crest cells and TUBB3/TH+ neurons were detected earlier (day 15) and more frequently than in the other conditions; secretion of ALB was found to be minimal.

Next, human iPSCs were differentiated as with conventional HLO culture, but with the addition of 10 μM SB431542 from days 3-6, i.e. when differentiating the definitive endoderm into foregut spheroids. TUBB3+ innervation appeared, but no TH signal was observed. CHAT, a parasympathetic marker, was detected in some TUBB3+ nerves.

Next, human iPSCs were differentiated as with conventional HLO culture, but with the addition of 250 nM of the BMP inhibitor LDN193189 and 3 μM CHIR from days 3-6, i.e. when differentiating the definitive endoderm into foregut spheroids. More neural crest cells and TUBB3+ neurons were detected than in HLO treated with LDN alone.

Next, human iPSCs were differentiated as with conventional HLO culture, but with the addition of 250 nM of the BMP inhibitor LDN193189 alone or in combination with 10 μM SB431542 from days 6-10, i.e. when differentiating the foregut spheroids. Neurons were sometimes detected, although the rate of neuronal appearance varies between batches. ALB secretion was about ⅓ lower.

Adding LDN/SB to Day 3-Day 6 is best in terms of the amount of sympathetic neurons, or sympathetic nerves, generated in the HLO. LDN 250 creates sympathetic neurons, or sympathetic nerves, reproducibly without inhibiting differentiation to the liver is not badly inhibited. Generating sympathetic neurons, or sympathetic nerves, reproducibly by adding CHIR3 μM and SB/LDN from Day 6 onwards also is a favorable system that can generate sympathetic neurons, or sympathetic nerves, while inducing the liver.

### Example 8. Determining Target Cells of the Sympathetic Nervous System within the HLO

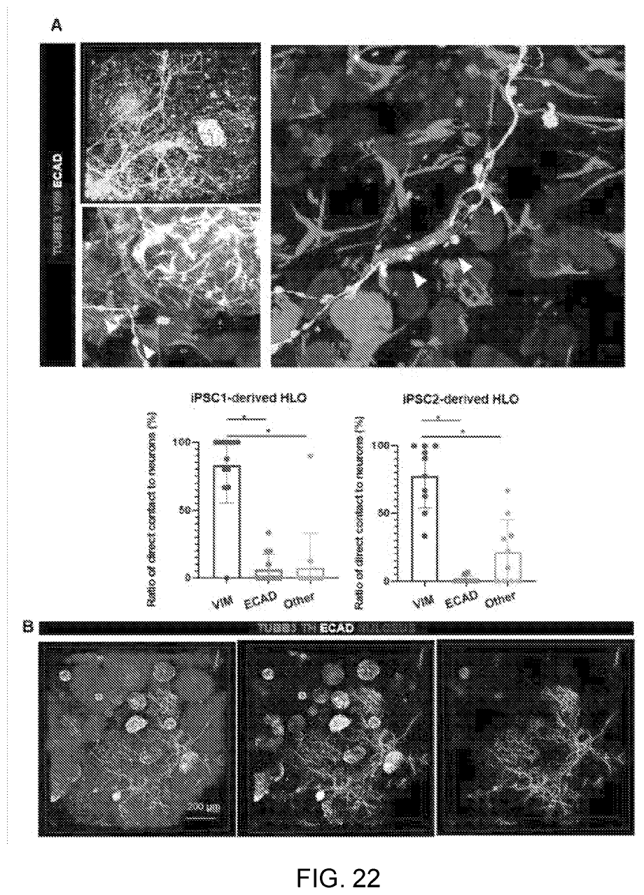

As neurotransmitters are degraded as soon as they are secreted extracellularly, it is thought that the sympathetic nervous system is affected by cells physically close to the nerve. Therefore, whole-mount staining of HLOs was used to identify cells in HLOs adjacent to sympathetic neurons, or sympathetic nerves. It is important to note that sympathetic neurons, or sympathetic nerves, are characterized by the fact that they secrete neurotransmitters from the tip of the nerve but also from a region in the nerve axon called the varicosity. The varicosity regions were observed in abundance in sympathetic nerve axon of HLOs, so in this verification, the adjacency between the nerve axon and other cells was assessed. The results showed that the majority of TUBB3+ nerves were adjacent to VIM positive hepatic stellate cells. These results are shown in FIG. 22A-B.

It is also clear from the localization of sympathetic neurons, or sympathetic nerves, in HLOs that sympathetic neurons, or sympathetic nerves, are localized in aggregates with a higher density of hepatic stellate cells than in the spheres, which is composed of hepatocytes. Results from physical location demonstrate that sympathetic neurons, or sympathetic nerves, target hepatic stellate cells rather than hepatocytes. TEM images of HLOs can be analyzed, and retrograde tracking of HLOs can be conducted using AAV2retro to further define the cells targeted by the sympathetic neurons, or sympathetic nerves, of HLOs.

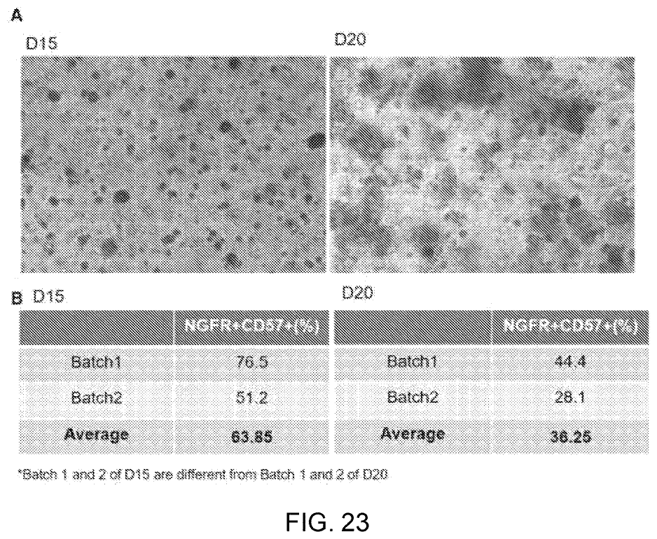

The involvement of specific cell types can also be determined at various stages of differentiation. From whole-mount staining data, it has been observed that the cells of the glowing spheres are composed of epithelial lineages, while the other aggregates are mainly hepatic stellate cells and neural lineages (FIG. 22B and FIG. 23).

Based on flow cytology data on neural crest cells and previous data, human iPS cell-derived Day 15 organoids consist of approximately 20% epithelial cells, 60% neural crest cells and the rest hepatic stellate cells and hematopoietic cells, while in day 20 organoids, epithelial cells account for approximately 20-30%, with 30% neural cells (including neural crest cells), and the remainder consisting of hepatic stellate cells and hematopoietic cells.

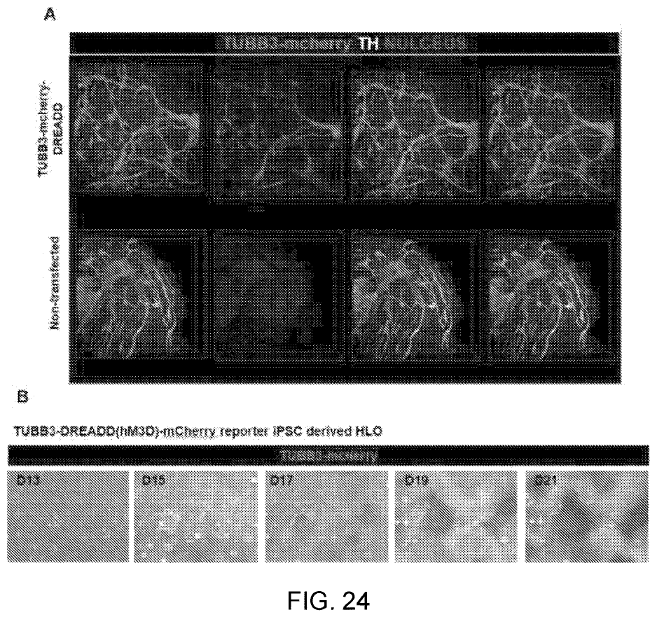

In organoids established from reporter iPS cells in which TUBB3-positive cells glowed with the fluorescent substance mCherry, mCherry expression overlapped with TH. When the reporter line was used to follow the mcherry-expressing cells in time series from day 13 to HLO, mcherry-expressing cells started to be detected from day 17 and were enriched in liver stellate cells in abundant aggregates (FIG. 24A-B).

### Example 9. Methods of Using Sympathetic Neuron-Containing Liver Organoids

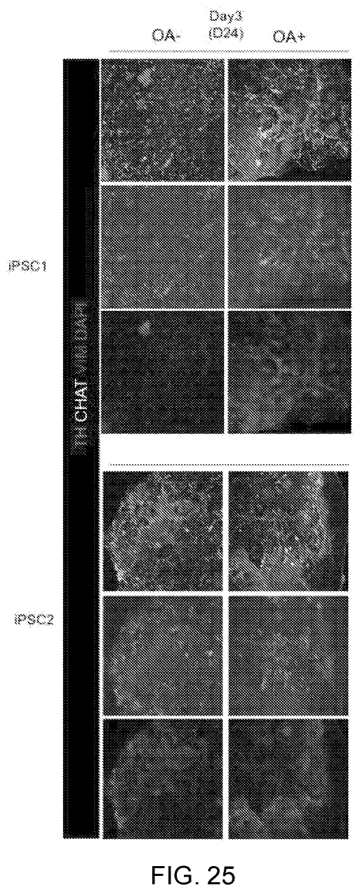

1. The distribution of sympathetic nerves projected to the liver is very different in humans as compared to experimental model animals, such as mice and rats. The presently described sympathetic neuron-containing human liver organoid model is expected to elucidate the distinctive mechanisms and timing of intrahepatic sympathetic projections during human liver development.
2. Although it has been reported that sympathetic nerves contribute to the development and function of multiple organs, little is known regarding sympathetic nerves in the liver. This model leads to the identification of sympathetic effects on human liver development and the elucidation of their mechanisms of action. The model can also enable construction of enhanced (or mature) liver organoids by artificially regulating sympathetic nerve activity.
3. The sympathetic nervous system has been reported to regulate gluconeogenesis in humans, but the mechanism by which it is promoted is unknown. Using this model, the mechanisms of human hepatic sympathetic innervation in gluconeogenesis can be elucidated.
4. Patients with diabetes and NASH have been shown to have more active sympathetic nerves than healthy individuals, but the role of human intrahepatic sympathetic nerves in these diseases is unknown. This model will allow us to identify the role of sympathetic nerves and their mechanisms in liver diseases. Also, in a high-fat diet mouse model, hepatic sympathetic innervation has been shown to increase and project to hepatocytes, and similarly in our NASH model, innervation increased, as shown in FIG. 25. The sympathetic neuron-containing human liver organoid model can therefore identify the features and functions of sympathetic nerves that are altered by liver disease.
5. The lack of sympathetic projection to the transplanted liver increases the risk of liver transplant patients developing diabetes. Based on this background, liver organoids, composed of cells with the ability to induce nerves and neuroprojection, are expected to be an alternative liver tissue for liver transplantation to reduce the risk of developing diabetes.
6. The sympathetic neuron-containing human liver organoid model can also lead to the establishment of drug screening systems for nerve-targeted liver diseases (such as, for example, MASH).

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A liver organoid, wherein the liver organoid comprises hepatic stellate cells, hepatocytes, and sympathetic neurons.

2.-4. (canceled)

5. The liver organoid of claim 1, wherein the sympathetic neurons further comprise secretory vesicles.

6. The liver organoid of claim 5, wherein the secretory vesicles store and secrete neurotransmitters.

7.-10. (canceled)

11. The liver organoid of claim 1, wherein the sympathetic neurons produce one or more sympathetic nervous system neurotransmitter and/or one or more noradrenaline synthesizing enzymes.

12. (canceled)

13. The liver organoid of claim 1, wherein the sympathetic neurons activate β-adrenergic receptor signaling in hepatic stellate cells.

14. (canceled)

15. The liver organoid of claim 1, wherein the hepatic stellate cells in liver organoid express higher levels of β-adrenergic receptor genes and lower levels of α2-adrenergic receptor genes than other cells.

16.-17. (canceled)

18. The liver organoid of claim 1, wherein the liver organoid comprises spheres of epithelial cells and aggregates of mesenchymal cells.

19.-20. (canceled)

21. The liver organoid of claim 18, wherein cells self-assemble into the spheres of epithelial cells comprising hepatocytes and aggregates of mesenchymal cells comprising hepatic stellate cells and sympathetic neurons.

22. The liver organoid of claim 18, wherein the spheres of epithelial cells comprising hepatocytes and aggregates of mesenchymal cells comprising hepatic stellate cells and sympathetic neurons self-assemble into the liver organoid containing sympathetic neurons.

23.-30. (canceled)

31. The liver organoid of claim 1, wherein the liver organoid performs spontaneous neural activity.

32. The liver organoid of claim 1, wherein the liver organoid has a fatty liver phenotype.

33.-36. (canceled)

37. The liver organoid of claim 1, wherein the liver organoid is differentiated from pluripotent stem cells.

38.-41. (canceled)

42. A method of producing the liver organoid containing sympathetic neurons, the method comprising:
a) activating an FGF signaling pathway and a Wnt signaling pathway, and optionally inhibiting a BMP signaling pathway, in definitive endoderm cells (DE), for a first period of time;
b) activating an FGF signaling pathway, a Wnt signaling pathway, and a retinoic acid (RA) signaling pathway, and optionally inhibiting a BMP signaling pathway, in the cells of step a), for a second period of time, thereby differentiating the DE to posterior foregut cells; and
c) embedding the posterior foregut cells in a basement membrane matrix, and optionally inhibiting a BMP signaling pathway in the embedded posterior foregut cells for a third period of time; and
d) culturing the posterior foregut cells for a fourth period of time to differentiate the posterior foregut cells to liver organoids;
wherein a BMP signaling pathway is inhibited in step b) and/or step c).

43.-78. (canceled)

79. A method of treating a liver-related disease or disorder, the method comprising:
transplanting, into a subject having a liver-related disease or disorder, the liver organoid of claim 1.

80.-88. (canceled)

89. Use of the liver organoid according to claim 1, as an in vitro human model system for studying hepatocyte function and developmental divergence; studying liver-related disease; identifying therapeutic targets; and/or identifying therapeutic compounds and/or compositions effective in treating a liver-related disease or disorder.

90.-91. (canceled)

92. A method of making a liver organoid having a reduced number of, or which is free of, sympathetic neurons, the method comprising inhibiting sympathetic neuron survival in a liver organoid comprising sympathetic neurons, wherein said inhibition of sympathetic neuron survival ablates the sympathetic neurons from the liver organoid, thereby making a liver organoid having a reduced number of, or which is free of, sympathetic neurons.

93.-104. (canceled)

105. A method of making a fatty liver organoid comprising a fatty liver phenotype, the method comprising providing the liver organoid of claim 1 with an inhibitor of sympathetic neuron survival and one or more fatty acids, thereby inducing the fatty liver phenotype in the liver organoid.

106.-129. (canceled)

130. A method of screening for a compound or composition for the treatment of fatty liver disease, the method comprising:
contacting the liver organoid of claim 1 comprising a fatty liver phenotype with the compound or composition; and
detecting a change in the fatty liver phenotype of the fatty liver organoid.

131.-168. (canceled)

169. A kit comprising means for performing the method according to claim 40.

170.-173. (canceled)

174. The liver organoid of claim 21, wherein there is an observable and/or measurable boundary between the spheres of epithelial cells and aggregates of mesenchymal cells.
